(19) United States
(12) Patent Application Publication
(10) Pub. No.: US 2021/0325409 A1
(43) Pub. Date: Oct. 21, 2021

(54) BIOMARKERS AND USES THEREOF FOR DIAGNOSING THE SILENT PHASE OF ALZHEIMER'S DISEASE

(71) Applicant: AgenT, Evry-Courcouronnes (FR)

(72) Inventors: Jérôme BRAUDEAU, Paris (FR); Baptiste BILLOIR, Paris (FR); Benoît SOUCHET, Paris (FR); Alkéos Michaïl, Paris (FR)

(21) Appl. No.: 17/237,654

(22) Filed: Apr. 22, 2021

Related U.S. Application Data
(63) Continuation of application No. PCT/EP2020/080324, filed on Oct. 28, 2020.

(30) Foreign Application Priority Data
Oct. 28, 2019 (EP) 19306400.3
Jun. 17, 2020 (EP) 20305661.9

(51) Int. Cl. G01N 33/68; G06N 20/00; G06N 3/04; G06K 9/62

(52) U.S. Cl. CPC G01N 33/6896; G06N 20/00; G06K 9/6256; G06K 9/6276; G06N 3/0454

(57) ABSTRACT

The present invention relates to a molecular signature of the silent phase of Alzheimer's disease; and to methods using the same, for diagnosing a silent stage of Alzheimer's disease in a subject, stratifying a silent phase of Alzheimer's disease in a subject into different grades of the silent phase, prognosticating the progress of a silent phase of Alzheimer's disease in a subject, and determining a personalized course of treatment in a subject affected with a silent phase of Alzheimer's disease, it also relates to a computer system comprising a machine learning algorithm trained for diagnosing a silent phase of Alzheimer's disease in a subject.

### CROSS-REFERENCE TO RELATED APPLICATION

This application is a Continuation of and claims priority to International Patent Application No. PCT/EP2020/080324, filed Oct. 28, 2020, which claims priority to EP Patent Application No. 20305661.9, filed on Jun. 17, 2020, which claims priority to EP Patent Application No. 19306400.3, filed Oct. 28, 2019, each of which is hereby incorporated by reference in its entirety.

### FIELD OF INVENTION

The present invention relates to a molecular signature of the silent phase of Alzheimer's disease; and to methods using the same, for diagnosing a silent stage of Alzheimer's disease in a subject, stratifying a silent phase of Alzheimer's disease in a subject into different grades of the silent phase, prognosticating the progress of a silent phase of Alzheimer's disease in a subject, and determining a personalized course of treatment in a subject affected with a silent phase of Alzheimer's disease. It also relates to a computer system comprising a machine learning algorithm trained for diagnosing a silent phase of Alzheimer's disease in a subject.

### BACKGROUND OF INVENTION

Alzheimer's disease (AD) is the most frequent cause of dementia in the Western world. In clinical terms, AD is characterized by a progressive cognitive decline that usually begins with memory impairment. As the disease progresses, AD inevitably affects all intellectual functions including executive functions, leading to complete dependence for basic activities of daily life and premature death. Around 50 million people live with AD worldwide and the number of patients is estimated to surge to 131.5 million by 2050 if we don't find a cure (Prince et al, 2015 World Alzheimer Report 2015. The global impact of dementia: An analysis of prevalence, incidence, cost and trends (Rep.). London: Alzheimer's disease international (ADI)).

The current cost of the disease is about a trillion US dollars a year, and that's forecast to double by 2030. In the US, out-of-pocket costs for families affected with AD account for more than $8,000 on average each year. It makes AD the most expensive illness for families during the last five years of life (Kelley et al, 2013 J Gen Intern Med. 28(2):304-9). Unfortunately, there are no effective treatments against AD, although some drugs can alleviate the symptoms associated with it.

One century ago, Dr. Alois Alzheimer described the first AD patient Dr. Alois Alzheimer identified the cerebral lesions of the disease more than a century ago (Shampo et al., 2013. Mayo Clin Proc. 88(12):e155). His patient, Auguste Deter, was displaying progressive memory loss, impaired thinking, disorientation, and changes in personality. At a microscopic level, Dr. Alzheimer identified two main cerebral aggregates of the disease: the senile plaques and the neurofibrillary tangles. However, it was not until 1984 that researchers revealed that the main components of senile plaques were amyloid peptides resulting from amyloid precursor protein (APP) cleavage (McKhann et al, 1984. Neurology. 34(7):939-44). Only few years after these discoveries, neurofibrillary tangles were characterized as hyperphosphorylated Tau aggregates (Jellinger, 2006. J Neural Transm (Vienna). 113(11): 1603-23). These major discoveries marked the beginning of more than three decades of intensive research.

Despite 30 years of intensive research, almost 100% of clinical trials have failed

As of today, two main events of AD are well established. AD is characterized by a progressive accumulation of b-amyloid peptide (Ab) that leads to a gradual Tau hyperphosphorylation. Consequently, the patients display a progressive decline of their cognitive functions that is followed by senile plaques deposition and fibrillary tangles formation. At the ultimate stage, dementia appears, in an events sequence known as the “amyloid cascade” (FIG. 1).

The neurological assessment of the patient and concurrent diagnosis is made only after the first signs of dementia have appeared. Despite billions of dollars invested in R&D to find an effective treatment, AD clinical trials still have the lowest success rate of any disease area—less than 1% compared with 19% for cancer (Cummings et al, 2017. Alzheimers Dement (N Y). 3(3):367-384). This high failure rate is attributed to the “too late” stage targeted during clinical trials (i.e., the dementia stage), to a lack of fundamental knowledge of the disorder and to current animal models which do not fully replicate the human AD course. In particular, the pathophysiological link between APP processing (including soluble Aβ peptides production) and Tau pathology remains challenging in AD animal models.

Therefore, the lack of animal models mimicking the key events observed in human AD raises the question of the validity of the modelling technologies used.

No early diagnosis, no possible salvation

Until recently, the diagnosis of AD was exclusively based on a neuropsychological assessment. Despite recent advances in biomarkers, their sensitivity and specificity remain insufficient.

The first biological signs of the disease appear at least 20 years before the clinical diagnosis (FIG. 2). Thus, the diagnosis is established when most of the damages have occurred to the brain and when the patient is already suffering from severe dementia (Sperling et al., 2014. Neuron. 84(3): 608-22), making the chances of successful treatment very low. However, it is impossible to identify silent AD biomarkers from diagnosed AD patients. Indeed, blood biomarkers evolve through the pathology's progress. It is thus impossible to presume the variations during the pre-symptomatic phase based on the variations from AD-diagnosed patients. This explains why the identification of biomarkers from the silent phase is so difficult, and why the scientists have failed to find an early diagnosis.

Currently, most of the biomarkers under investigations are of 3 main types and based on AD-diagnosed patient studies:

(1) cerebral amyloid-b imaging or blood Aβ42 measurement;

(2) cerebral Tau imaging or blood Tau measurement; or

(3) common biomarkers of all neurodegenerative disorders

(1) Cerebral Amyloid-b Imaging or Blood Ab42 Measurement

For example, Dr. Koichi Tanaka and his group have developed a powerful technology to measure the most amyloidogenic amyloid-b peptide (the Aβ42 peptide) in the blood in which the concentration of Aβ42 is known to be very low. This technology opens a new way to better identify people with cerebral amyloid-b plaque burden, thanks to a simple blood test. They seek to replace in a near future the costly and non-safety measurements of Aβ42 peptide which currently consist of in vivo imaging (PIB-PET) and cerebrospinal fluid biomarkers after a lumbar puncture.

However, this technology suffers major limitations as to its use as a suitable diagnosis tool for both the silent and late phases of AD.

First, the cerebral amyloid-b plaque burden is known to poorly correlate with the AD status. In the paper (Nakamura et al., 2018. Nature. 554(7691):249-254), the authors admitted: “In the NCGG data set, there were 9 out of 29 (31%) patients who had been diagnosed with AD but were PIB-PET Aβ and “a new clinical data set consisting of 31 AD (22 Aβ+ and 9 Aβ−, classified by PIB-PET) and 20 non-AD (8 Ab+ and 12 Aβ−) cases”. To summarize, ≈30% of AD patients are thus PIB-PET Aβ− and ≈40% of healthy individuals are PIB-PET Aβ+ (FIG. 3).

Nakamura et al. concludes stating that “These results demonstrate the potential clinical utility of plasma biomarkers in predicting brain amyloid-b burden at an individual level) but because of the lack of correlation between brain amyloid-b burden and the AD status, this technology is unable to precisely diagnose individuals suffering from AD.

Second, this technology does not measure the consequences of the other main pathology involved in AD: the tauopathy. With the same amyloid-b amount in the brain, someone will develop AD (including the tauopathy part) and someone will not, depending on their individual susceptibility to amyloid-b toxicity. The more “responsive” to amyloid-b peptide toxicity the individual is, the higher his probability to develop AD, and this, independently from the amyloid-b peptides amount (in brain, cerebrospinal fluid or blood).

(2) Cerebral Tau Imaging or Blood Tau Measurement

Cerebral Tau load is currently under investigations. However, due to a poor precision of Tau imaging, the aggregated Tau is only visible at the late stages of progression, when the number of tangles is huge. Tau imaging cannot be used as a silent phase biomarker.

Besides, Tau and phospho-Tau could only be measured in the blood after the neuronal cell death, because of their particular cellular localization. It thus constitutes a late phase biomarker and cannot be used to detect patients during the silent phase of AD (far before the atrophy appearance).

(3) Common Biomarkers of all Neurodegenerative Disorders

All those biomarkers are mainly identified through a priori approaches. This methodology limits the finding of new biomarkers unrelated to amyloid protein, neurotrophic factors (NFTs) or neuroinflammation biomarkers. It is important to keep in mind that amyloid protein blood concentration is poorly correlated to AD status (avoiding its use as AD diagnosis) and neurotrophic factors and neuroinflammation processes are both involved only in the clinical phase of AD. These biomarkers are, once again, irrelevant to detect patients during the silent phase of AD. Moreover, growth factors and neuroinflammation biomarkers are poorly specific of AD and cannot be used as a differential diagnosis of AD.

In order to find suitable biomarkers of the silent phase, it is therefore necessary to have faithful models of AD reproducing this asymptomatic phase. But transgenic animal models are not consistent with the human AD pathology.

Transgenic AD models' limitations reduce their ability to enable the development of a silent phase AD diagnosis

Most of AD models used in laboratories are transgenic mice expressing human mutated genes associated with familial forms of AD (such as amyloid protein precursor [APP], presenilin-1 [PSEN1], and presenilin-2 [PSEN2]). Because each of these mutations leads to an increased Aβ production, these models are pertinent to quickly mimic the amyloid plaques deposition in a very short time. In addition, they are suitable models to develop pertinent positron emission tomography (PET) or magnetic resonance imaging (MRI) tracers to identify senile plaques or neurofibrillary tangles in the brains of patients. However, these existing transgenic animal models have at least three main limitations.

First, several studies have shown that the development of AD hallmarks in transgenic mice depends on the expression of the transgene(s). Consequently, aging—which is the strongest risk factor for AD—is often ignored in AD studies because most of the mice models present an AD-like phenotype just in a few months. The fact that all these mice develop an accelerated senescence not similar to the human disease is the first limitation.

Second, no genetic mutations in the MAPT gene (encoding the Tau protein) have been found in AD patients. Thereby, mice models have been developed using MAPT mutations found in a subset of tauopathies to develop neurofibrillary tangles. Crossings between several lines have been performed to generate transgenic models developing both amyloid and tau pathologies, such as the 3×Tg-AD mouse (Duyckaerts et al, 2008. Acta Neuropathol. 115(1):5-38). But in the human disease, both pathologies appear independently: Aβ, which is a causative pathogenic factor based on the amyloid cascade, triggers the tau pathology. The amyloid cascade is not reproduced in these mice models, which represents a second limitation.

Third, the transgenes that are overexpressed in transgenic animals are not overexpressed in patients (except for the AD form developed by patients with Down syndrome), which is why the level of neurotoxic peptides—such as Aβ—is much higher in these transgenic models than in AD patients' brain (Audrain et al, 2016. Mol Neurodegener. 11:5). The last limitation is therefore the supra-pathological concentration of pathological metabolites expressed by transgenic AD models.

Furthermore, other modelling strategies have been developed, such as the injection-based animal models, induced by intracerebral injections of amyloid or tau peptides directly into the brain (Puzzo et al., 2017. Elife. 6.pii:e2699). Similar limitations to the transgenic models may also be addressed here. Despite these limitations, existing animal models of AD have provided numerous data that had led to the understanding of neurological AD lesions and the evaluation of various potential therapeutic strategies. Overall, the research community regrets the lack of adequate models. This absence of human-close AD models appears as a limiting factor for the development of diagnoses (Lecanu & Papadopoulos, 2013. Alzheimers Res Ther. 5(3): 17). In any cases, key factors including aging, influence of soluble Aβ peptides toward tau pathology and faithful clinical Aβ concentrations remain challenging and should be designed in adequate AD animal models.

Advent of Non-Transgenic Models which are Closer to the Human Pathology

In order to mimic the progression of the disease in an in vivo model and in a way that reproduces more faithfully the clinical observation, an innovative AD rat model, the AgenT rat, was recently developed through injection of adeno-associated viruses (AAV) encoding a human mutant APP protein and presenilin-1 (PS1) into the hippocampi of adult rodents (U.S. Pat. No. 10,159,227 and European patent EP3066203).

This model can be described as a disruptive technology and a time course closer to the human progression of AD.

Indeed, the technology used is not based on a transgenic approach. Because AD induction is conducted only on adult animals, the AgenT rat does not suffer from developmental compensation or genetic drift. Moreover, the pattern of APP expression in the AgenT rat may mimic the genomic mosaicism recently described in the sporadic form of human AD, in which an increase in copy number was observed for the APP gene in a limited subset of neurons (Bushman et al, 2015. Elife. 4) and an appearance of somatic mutations known to be associated with familial form of Alzheimer's disease was described (Lee et al, 2018. Nature. 563(7733):639-645). The AgenT rat could thus be considered as a closer model of the sporadic form of AD than transgenic animals.

Moreover, induced APP pathology appears similar to the human one in terms of the amount of amyloid peptide and Aβ42/40 ratio. The induced amyloid pathology leads to pathophysiological mechanisms including progressive Tau hyperphosphorylation. Slow progression of the APP pathology allows the progressive development of an endogenous Tau pathology to take place without the occurrence of a would-be interfering early inflammation and plaque formation. These steps could be considered as the silent phase of AD, beginning in patients at least 18 years before the current clinical diagnosis (Rajan et al., 2015. Neurology. 85(10):898-904). The next phase of AD disease progression consists of the appearance of AD-related cerebral lesions such as senile plaques, cerebral amyloid angiopathy and tangle-like aggregates, which only appear in aged AgenT rats.

All these features make the AgenT rat model a powerful tool to better predict blood biomarker behavior according to the stage of progression. This model thus constitutes a suitable study system to characterize new biomarkers or panel of biomarkers for the development of an early diagnosis.

It is in that sense that the Inventors have identified a panel of 119 best-in-class biomarkers suitable to predict AD, using artificial intelligence approaches. Surprisingly, the Inventors have been able to demonstrate that an artificial neural network, trained using data from AgenT rats (i.e., rats affected with AD but still asymptomatic) and healthy rats, was ultimately able to predict AD in its asymptomatic or silent phase, from a subset of about five biomarkers or less, randomly picked from the full list of 119 best-in-class biomarkers.

The Inventors have further been surprisingly able to demonstrate that the trained artificial neural network, using these random subsets of about five biomarkers or less, was not only able to predict AD in its silent phase, but to further stratify silent AD into different grades.

### SUMMARY

The present invention relates to a molecular signature of the silent phase of Alzheimer's disease, wherein said molecular signature comprises at least five biomarkers selected from the group of biomarkers of Table 1A.

In one embodiment, the molecular signature of the silent phase of Alzheimer's disease comprises the biomarkers of Table 10A, Table 10B, Table IOC or Table 10D.

The present invention further relates to a method for diagnosing a silent stage of Alzheimer's disease in a subject, comprising the steps of:

a) determining a molecular signature by measuring the level, amount or concentration of at least five biomarkers selected from the group of biomarkers of Table 1A, in a sample previously obtained from said subject,

b) comparing the molecular signature obtained at step a) with a reference signature, and

c) diagnosing the subject as being affected with a silent stage of Alzheimer's disease based on a correlation of the molecular signature with the reference signature.

The present invention further relates to a method of prognosticating the progress of a silent phase of Alzheimer's disease in a subject, comprising the steps of:

a) determining a molecular signature by measuring the level, amount or concentration of at least five biomarkers selected from the group of biomarkers of Table 1A, in a sample obtained from said subject,

b) comparing the molecular signature obtained at step a) with a reference signature, and

c) prognosticating the progress of Alzheimer's disease, based on a correlation of the molecular signature with the reference signature.

The present invention further relates to a method of determining a personalized course of treatment in a subject affected with a silent phase of Alzheimer's disease, comprising the steps of:

a) determining a molecular signature by measuring the level, amount or concentration of at least five biomarkers selected from the group of biomarkers of Table 1A, in a sample obtained from said subject,

b) comparing the molecular signature obtained at step a) with a reference signature, and

c) determining the personalized course of treatment for the subject, based on a correlation of the molecular signature with the reference signature.

The present invention further relates to a method of stratifying a silent phase of Alzheimer's disease in a subject into different grades of the silent phase, preferably into SI, S2 or S3 grades, comprising the steps of:

a) determining a molecular signature by measuring the level, amount or concentration of at least five biomarkers selected from the group of biomarkers of Table 1A, in a sample obtained from said subject,

b) comparing the molecular signature obtained at step a) with a reference signature, and

c) stratifying the subject into a grade of the silent phase of Alzheimer's disease, based on a correlation of the molecular signature with the reference signature.

In a particular embodiment of the method of stratifying a silent phase of Alzheimer's disease in a subject into different grades of the silent phase, the molecular signature comprises at least 14 biomarkers selected from the group of biomarkers of Table 1A.

In one embodiment, the reference signature comprises the level, amount or concentration of the same at least five biomarkers measured in a sample previously obtained from a substantially healthy subject, preferably measured in samples previously obtained from a population of substantially healthy subjects.

In one embodiment, the correlation at step c) is measured by comparing the variation of level, amount or concentration of the at least five biomarkers in the molecular signature and in the reference signature with the biomarker variation profile of Table 3.

In one embodiment, the molecular signature comprises the biomarkers of Table 10A, Table 10B, Table IOC or Table 10D.

In one embodiment, the comparison at step b) is conducted using at least one machine learning algorithm.

In one embodiment, said at least one machine learning algorithm is selected from the group comprising an artificial neural network (ANN), a perceptron algorithm, a deep neural network, a clustering algorithm, a k-nearest neighbors algorithm (k-NN), a decision tree algorithm, a random forest algorithm, a linear regression algorithm, a linear discriminant analysis (LDA) algorithm, a quadratic discriminant analysis (QDA) algorithm, a support vector machine (SVM), a Bayes algorithm, a simple rule algorithm, a clustering algorithm, a meta-classifier algorithm, a Gaussian mixture model (GMM) algorithm, a nearest centroid algorithm, an extreme gradient boosting (XG Boost) algorithm, a linear mixed effects model algorithm, and a combination thereof.

In one embodiment, the at least one machine learning algorithm is trained with a training dataset comprising information relating to the level, amount or concentration of the same at least five biomarkers of Table 1A from samples previously obtained from substantially healthy subject and from subjects known to be affected with a silent stage of Alzheimer's disease.

In one embodiment, the at least one machine learning algorithm is trained with a training dataset comprising the biomarker variation profile of Table 3.

The present invention further relates to a computer system for diagnosing a silent phase of Alzheimer's disease in a subject, the computer system comprising:

(i) at least one processor, and

(ii) at least one storage medium that stores at least one code readable by the processor, and which, when executed by the processor, causes the processor to:

a. receive an input level, amount or concentration of at least five biomarkers selected from the group of biomarkers of Table 1A, determined in a sample previously obtained from said subject,

b. analyze and transform the input level, amount or concentration of the at least five biomarkers by organizing and/or modifying each input level, amount or concentration to derive a probability score and/or a classification label via a machine learning algorithm,

wherein the machine learning algorithm is trained with a training dataset, wherein the training dataset comprises information relating to the level, amount or concentration of the same at least five biomarkers of Table 1A from samples previously obtained from subjects of known Alzheimer's disease status,

c. generate an output, wherein the output is the classification label or the probability score, and

d. provide a diagnosis of the subject as being affected or not with a silent stage of Alzheimer's disease based on the output.

The present invention further relates to a computer-implemented method for diagnosing a silent phase of Alzheimer's disease in a subject, said method comprising:

a. receiving an input level, amount or concentration of at least five biomarkers selected from the group of biomarkers of Table 1A, determined in a sample previously obtained from said subject,

b. analyzing and transforming the input level, amount or concentration of the at least five biomarkers by organizing and/or modifying each input level, amount or concentration to derive a probability score and/or a classification label via a machine learning algorithm,

wherein the machine learning algorithm is trained with a training dataset, wherein the training dataset comprises information relating to the level, amount or concentration of the same at least five biomarkers of Table 1A from samples previously obtained from subjects of known Alzheimer's disease status,

c. generate an output, wherein the output is the classification label or the probability score, and

d. provide a diagnosis of the subject as being affected or not with a silent stage of Alzheimer's disease based on the output.

In one embodiment, the training dataset comprises information relating to the level, amount or concentration of the same at least five biomarkers of Table 1A from samples previously obtained from substantially healthy subject and from subjects known to be affected with a silent stage of Alzheimer's disease.

In one embodiment, providing a diagnosis at step d. comprises providing a stratification of the subject being affected with a silent stage of Alzheimer's disease into a grade of said silent phase of Alzheimer's disease, preferably into a SI, S2 or S3 grade.

In a particular embodiment where step d. comprises providing a stratification, step a. comprises receiving an input level, amount or concentration of at least 14 biomarkers selected from the group of biomarkers of Table 1A.

In one embodiment, the training dataset comprises the biomarker variation profile of Table 3.

The present invention further relates to a computer program comprising software code readable by a processor adapted to perform, when executed by said processor, the computer-implemented method according to the present invention.

The present invention further relates to a non-transitory computer-readable storage medium comprising code which, when executed by a computer, causes a processor to carry out the computer-implemented method according to the present invention.

### DETAILED DESCRIPTION

The present invention relates to a molecular signature or profile of the silent phase of Alzheimer's disease.

As used herein, the terms “silent phase/stage” “pre-dementia phase/stage”, or “preclinic phase/stage”, when referring to Alzheimer's disease, are interchangeable and refer to a preclinical state of subjects who are yet cognitively unimpaired but display at least one of the Alzheimer's features: soluble Aβ peptides dysregulation, increase of hyperphosphorylated Tau protein, appearance of senile plaques and tangles. These terms encompass both the “asymptomatic phase” and the “prodromal phase” of Alzheimer's disease. The “silent phase” spans from the first molecular events {i.e., dysregulation of Aβ peptides production or clearance) to the onset of the first clinical symptoms of Alzheimer's disease in a subject. For a detailed definition, see Dubois et al, 2016 {Alzheimers Dement. 12(3):292-323) or Sperling et al, 2011 {Alzheimers Dement. 7(3):280-292), the content of which is herein incorporated by reference in its entirety.

As used herein, the terms “asymptomatic phase/stage” or “presymptomatic phase/stage”, when referring to Alzheimer's disease, are interchangeable and refer to a preclinical state of subjects who are yet cognitively unimpaired but display at least one of the Alzheimer's features at the brain level: soluble Aβ peptides dysregulation, increase of hyperphosphorylated Tau protein, and, in some cases, appearance of senile plaques and tangles. These subjects will develop Alzheimer's clinical symptoms several years or decades later (Hubbard et al, 1990. Neuropathol Appl Neurobiol. 16(2): 111-21).

At this stage of the pathology, the cerebral alterations are exclusively molecular. The patient, although sick in practical terms, does not present any objective cognitive disorder. Cerebrospinal fluid (CSF) biomarkers and PET imaging biomarkers are typically negative.

As used herein, the terms “prodromal phase/stage” or “mild cognitive impairments (MCI) stage/phase”, when referring to Alzheimer's disease, are interchangeable and refer to the stage between the first cognitive abnormalities (abnormal regarding the normal aging cognitive decline) and the onset of dementia symptoms. It is characterized by problems with memory, language, thinking or judgment, but no symptoms of AD dementia. The cerebral concentration of amyloid peptides continues to increase while the CSF concentration tends to decrease. However, the basic level varies from one person to another. This explains why 32% of cognitively normal people exceed the threshold of positivity for amyloid while 35% to 52% of prodromal patients are negative (Landau, 2020 July 28. Imaging biomarkers and Alzheimer's disease prevention. Speech presented at the Alzheimer's Association International Conference (AAIC) 2020, online). The amyloid concentration is thus not specific enough to identify Alzheimer's patients. Some patients will, at this stage, begin to show an increase in the concentration of Tau protein both in the brain and at the peripheral level (CSF, blood). However, it remains low and does therefore not allow to diagnose all prodromal Alzheimer's patients.

The terms “clinical symptoms”, “symptoms of the clinical phase”, “AD dementia”, “dementia due to Alzheimer's disease”, “symptoms of AD dementia”, when referring to Alzheimer's disease, refers, without limitation, to symptoms spanning from memory loss that disrupts daily life, challenges in planning or solving problems, difficulty completing familiar tasks at home, at work or at leisure, confusion with time or place, trouble understanding visual images and spatial relationships, new problems with words in speaking or writing, misplacing things and losing the ability to retrace steps, decreased or poor judgment, withdrawal from work or social activities, or changes in mood and personality. Such clinical symptoms are described, e.g, on the Alzheimer's Association website at www.alz.org/alzheimers-dementia/10_signs.

In one embodiment, the molecular signature or profile of the invention comprises biomarkers whose mean profile of level, amount or concentration is characteristic of the silent phase of Alzheimer's disease, when taking in comparison to a reference signature or profile.

By “is/are/being characteristic”, when referring to the levels, amounts or concentrations of biomarkers, it is meant that the level, amount or concentration of a given biomarker—or that the mean profile of biomarkers' level, amount or concentration—is substantially different or substantially similar to the level, amount or concentration of the same biomarker—or to the mean profile of biomarkers' level, amount or concentration—from a reference subject. Whether “characteristic” should be understood as being “substantially different” or “substantially similar” depends on the reference subject and its disease status.

In one embodiment, the level, amount or concentration of a given biomarker is “substantially different” if it is more than about 1% higher, 2% higher, 3% higher, 4% higher, 5% higher, 6% higher, 7% higher, 8% higher, 9% higher, 10% higher,

15% higher, 20% higher, 25% higher, 30% higher, 35% higher, 40% higher, 45% higher, 50% higher or more; or if it is more than about 1% lower, 2% lower, 3% lower, 4% lower, 5% lower, 6% lower, 7% lower, 8% lower, 9% lower, 10% lower, 15% lower, 20% lower, 25% lower, 30% lower, 35% lower, 40% lower, 45% lower, 50% lower or more than the level, amount or concentration of the same biomarker in a reference subject. In one embodiment, the level, amount or concentration of a given biomarker is “substantially different” if it is more than about 5% higher or 5% lower than the level, amount or concentration of the same biomarker in a reference subject.

In one embodiment, the level, amount or concentration of a given biomarker is “substantially similar” if it is less than about 1% higher, 2% higher, 3% higher, 4% higher, 5% higher, 6% higher, 7% higher, 8% higher, 9% higher, 10% higher, 15% higher, 20% higher, or more; or if it is less than about 1% lower, 2% lower, 3% lower, 4% lower, 5% lower, 6% lower, 7% lower, 8% lower, 9% lower, 10% lower, 15% lower, 20% lower, or more than the level, amount or concentration of the same biomarker in a reference subject. In one embodiment, the level, amount or concentration of a given biomarker is “substantially similar” if it is less than about 5% higher or 5% lower than the level, amount or concentration of the same biomarker in a reference subject.

In one embodiment, the levels, amounts or concentrations of biomarkers may be measured by methods well known in the art. Such method include, but are not limited to, mass spectrometry (such as, e.g, tandem mass spectrometry [MS/MS], chromatography-assisted mass spectrometry and combinations thereof), immunohistochemistry, multiplex methods (Luminex), western blot, enzyme-linked immunosorbent assay (ELISA), sandwich ELISA, fluorescent-linked immunosorbent assay (FLISA), enzyme immunoassay (EIA), radioimmunoassay (RIA), RT-PCR, RT-qPCR, Northern Blot, hybridization techniques (such as, e.g, use of microarrays, and combination thereof including but not limited to, hybridization of amplicons obtained by RT-PCR, sequencing such as, for example, next-generation DNA sequencing (NGS) or RNA-seq (also known as “whole transcriptome shotgun sequencing”)), and the like. In one embodiment, the molecular signature or profile of the invention comprises biomarkers whose levels, amounts or concentrations are characteristic of the grade SI of the silent phase of Alzheimer's disease, the grade S2 of the silent phase of Alzheimer's disease and/or the grade S3 of the silent phase of Alzheimer's disease, when taking in comparison to a reference signature or profile.

In one embodiment, the silent phase of Alzheimer's disease is defined as grade SI silent phase of Alzheimer's disease. The present invention relates thus to a molecular signature or profile of the grade SI of silent phase of Alzheimer's disease.

As used herein, the term “grade SI silent phase of Alzheimer's disease” or “grade SI” refers to that grade of the silent phase of Alzheimer's disease where the subjects exhibits no clinical symptoms such as mild cognitive impairment (MCI) and dementia, but where physiopathological features are observable. Such physiopathological features of grade SI include at least one of cerebral soluble Aβ42 concentration dysregulation and anxiety-like syndrome. Physiopathological features of grade SI do not include those of grade S2 and/or of grade S3 as defined hereafter.

In one embodiment, the silent phase of Alzheimer's disease is defined as grade S2 silent phase of Alzheimer's disease. The present invention relates thus to a molecular signature or profile of the grade S2 of silent phase of Alzheimer's disease.

As used herein, the term “grade S2 silent phase of Alzheimer's disease” or “grade S2” refers to that grade of the silent phase of Alzheimer's disease where the subjects exhibits no clinical symptoms such as mild cognitive impairment (MCI) and dementia, but where physiopathological features are observable. Such physiopathological features of grade S2 include those of grade SI, plus at least one of accumulation of soluble Aβ₄₂ peptides, hyperphosphorylation of Tau and accelerated forgetting. Physiopathological features of grade S2 do not include those of grade S3 as defined hereafter.

In one embodiment, the silent phase of Alzheimer's disease is defined as grade S3 silent phase of Alzheimer's disease. The present invention relates thus to a molecular signature or profile of the grade S3 of silent phase of Alzheimer's disease.

As used herein, the term “grade S3 silent phase of Alzheimer's disease” or “grade S3” refers to that grade of the silent phase of Alzheimer's disease where the subjects exhibits no clinical symptoms such as dementia, but where physiopathological features are observable. Such physiopathological features of grade S3 include those of grade SI and of grade S2, plus at least one of increase of hyperphosphorylated Tau, senile plaques, tangles and mild or strong memory impairments. In some cases, mild cognitive impairment could be considered as grade S3 symptoms.

FIG. 1 summarizes these three grades of the silent phase of Alzheimer's disease.

In one embodiment, the molecular signature or profile of the silent phase of Alzheimer's disease, of grade SI, of grade S2 and/or of grade S3 comprises at least 1 biomarker selected from the group of biomarkers of Table 1A. In one embodiment, the molecular signature or profile of the silent phase of Alzheimer's disease, of grade SI, of grade S2 and/or of grade S3 comprises 1 biomarker selected from the group of biomarkers of Table 1A. In one embodiment, the molecular signature or profile of the silent phase of Alzheimer's disease, of grade SI, of grade S2 and/or of grade S3 consists of 1 biomarker selected from the group of biomarkers of Table 1A.

TABLE 1A
BIOMARKERS OF THE SILENT PHASE OF ALZHEIMER'S DISEASE

| | |
|---|---|
| 10 kDa heat shock protein, mitochondrial | Glucose |
| 14-3-3 proteins | Glycosylphosphatidylinositol specific |
| | phospholipase D1 |
| 1-methyladenosine | HGF activator |
| 1-methyl-5-imidazoleacetate | Histidine-rich glycoprotein |
| 3,4-dihydroxybutyrate | Hyaluronidase |
| 3-amino-2-piperidone | Hydroxyproline |
| 3-methyl-2-oxobutyrate | Ighg protein |
| 4-methyl-2-oxopentanoate | Integrin beta |
| 5-methyl-2′-deoxycytidine | Integrin subunit alpha V |
| 5-methylthioadenosine (MTA) | Interleukin 1 receptor accessory protein |
| 5-oxoproline | Keratin, type II cytoskeletal 5 |
| Adenylate kinase 4, mitochondrial | Kininogen family |
| Allantoic acid | Lipase |
| Alpha-1B-glycoprotein | Lumican |
| Alpha-actinin-1 | Lysine and conjugates |
| Alpha-soluble NSF attachment protein | Lysyl oxidase-like 1 |
| Anserine | Macrophage migration inhibitory factor |
| Anti-F4/80 kappa light chain variable | Major urinary protein |
| region | |
| Apolipoproteins | Malate dehydrogenase, cytoplasmic |
| Arabonate/xylonate | Mannan-binding lectin serine peptidase 2 |
| Arp2/3 complex proteins | Mannose-binding protein A |
| Biotinidase | Microfibril-associated glycoprotein 4 |
| BWK3 | Multiple inositol polyphosphate |
| | phosphatase 1 |
| C4b-binding protein beta chain | Myosin regulatory light polypeptide 9 |
| Calpain small subunit 1 | Myosin regulatory light chain RLC-A |
| Calreticulin | N-acetylalanine |
| Carboxyesterase 1 family | N-acetylasparagine |
| Carboxypeptidase B2 | Octadecanedioate (C18) |
| Carnitine and conjugates | Oleate/vaccenate (18:1) |
| Carnosine | Palmitate (16:0) |
| Cholate and conjugates | Palmitoleate (16:1n7) |
| Chromogranin A | Peptidyl-prolyl cis-trans isomerase A |
| Clathrin heavy chain | Peptidyl-prolyl cis-trans isomerase |
| | FKBF1A |
| Coactosin-like protein | Peroxiredoxin 3 |
| Coagulation factor family | Phenylalanine |
| Complement system family | Polyubiquitin-C |
| Creatine | Prolylhydroxyproline |
| Creatinine | Proteasome complex family |
| Creatinine kinase family | Rat MHC class I truncated cell surface |
| | antigen mRNA |
| Cysteine-glutathione disulfide | Retinoic acid receptor responder 2 |
| Dimethylarginine | Retinol-binding protein |
| Dimethyl sulfone | Ribonate (ribonolactone) |
| EGF-containing fibulin-like extracellular | Ribulonate/xylulonate/lyxonate |
| matrix protein 1 | |
| EH domain-containing protein 3 | Sacsin molecular chaperone |
| Elongation factor 1-alpha | Serpin superfamily members |
| Ergothioneine | Serum amyloid P-component |
| Erythronate | Sphingosine 1-phosphate |
| Ethylmalonate | Sulfate |
| Extracellular matrix protein 1 | Superoxide dismutase [Mn], |
| | mitochondrial |
| F-actin-capping protein subunit alpha-2 | Talin 2 |
| Fibronectin 1 | Tartronate (hydroxymalonate) |
| Fibulin-1 | Thioredoxin |
| Fibulin-5 | Tmprss13 protein |
| Fructose | Transferrin receptor protein 1 |
| Fructose-bisphosphate aldolase | Transthyretin |
| FYN-binding protein 1 | Urinary protein 1 |
| Gelsolin | Valerate and conjugates |
| Globin family | Voltage-dependent anion-selective |
| | channel protein 3 |
| Globulin family | Xaa-Pro aminopeptidase 2 |
| Glucuronate | |

In one embodiment, the molecular signature or profile of the silent phase of Alzheimer's disease, of grade SI, of grade S2 and/or of grade S3 does not comprise at least 1 biomarker, such as, at least 2, 3, 4, 5, 6, 7, 8, 9, 10, 11, 12, 13, 14 or 15 biomarkers selected from the group comprising or consisting of 1-methyladenosine, 3,4-dihydroxybutyrate, 3-amino-2-piperidone, 4-methyl-2-oxopentanoate, arabonate/xylonate, creatine, creatinine, cysteine-glutathionedisulfide, dimethyl sulfone, erythronate, glucose, N-acetylalanine, sphingosine 1-phosphate and tartronate (hydroxymalonate).

As used herein, the term “14-3-3 proteins” refers to any one or more of the following proteins: 14-3-3 protein beta/alpha, 14-3-3 protein gamma, 14-3-3 protein epsilon, 14-3-3 protein zeta/delta, 14-3-3 protein eta, and 14-3-3 protein theta.

As used herein, the term “apolipoproteins” refers to any one or more of the following proteins: apolipoprotein A-I, apolipoprotein A-II, apolipoprotein A-IV, apolipoprotein B-100, apolipoprotein C-I, apolipoprotein C-II (Predicted), apolipoprotein C-III, apolipoprotein C-IV, apolipoprotein D, apolipoprotein E, rat apolipoprotein E protein, apolipoprotein H (beta-2-glycoprotein I), apolipoprotein M, and apolipoprotein N.

As used herein, the term “Arp2/3 complex proteins” refers to any one or more of the following proteins: actin-related protein 2, actin-related protein 2/3 complex subunit IB, actin-related protein 2/3 complex subunit 3, actin-related protein 2/3 complex subunit 4, actin-related protein 2/3 complex subunit 5, actin-related protein 3, and arp2/3 complex 34 kDa subunit.

As used herein, the term “carboxyesterase 1 family” refers to any one or more of the following proteins: carboxylesterase 1, carboxylesterase 1C, and carboxylesterase IE.

As used herein, the term “carnitine and conjugates” refers to any one or more of the following molecules: 2-methylbutyrylcarnitine (C5), acetylcarnitine (C2), arachidonoyl carnitine (C20:4), butyrylcarnitine (C4), carnitine, cis-4-decenoylcarnitine (00:1), isobutyryl carnitine (C4), isovalerylcarnitine (C5), laurylcarnitine (02), linoleoylcarnitine (08:2), myristoylcarnitine (04), octanoylcarnitine (C8), oleoylcarnitine (C 18), palmitoleoylcarnitine (06:1), palmitoylcamitine (06), propionyl carnitine (C3), stearoylcamitine (08), (S)-3-hydroxybutyrylcarnitine and deoxycamitine.

As used herein, the term “cholate and conjugates” refers to any one or more of the following molecules: chenodeoxycholate, cholate, deoxycholate, glycocholate, taurochenodeoxycholate, taurocholate, and taurodeoxy cholate.

As used herein, the term “coagulation factor family” refers to any one or more of the following proteins: coagulation factor V, coagulation factor IX, coagulation factor VII, coagulation factor X, coagulation factor XI, coagulation factor XII, coagulation factor XIII A chain, and coagulation factor XIII B chain.

As used herein, the term “complement system family” refers to any one or more of the following proteins: complement factor B, complement Clq subcomponent subunit A, complement Clq subcomponent subunit B, complement Clq subcomponent subunit C, complement Clr subcomponent, complement Clr subcomponent-like protein, complement Cls subcomponent, complement Cls subcomponent, complement C2, complement C3, complement C4, complement C4A, complement C4B, C4B-binding protein alpha chain, C4B-binding protein beta chain, complement C4-like, complement C5, complement C6, complement C7, complement C8 alpha chain, complement component C8 beta chain, complement C8 gamma chain, complement component C9, complement factor D, complement factor H, complement factor H-related protein, complement factor H-related protein 1, complement factor H-related protein 2, complement factor H-related protein 3, complement factor H-related protein 4, and complement factor I.

As used herein, the term “creatine kinase family” refers to any one or more of the following proteins: creatine kinase B-type, and creatine kinase M-type.

As used herein, the term “globin family” refers to any one or more of the following proteins: globin a2, globin a4, globin c2, globin c3, globin dl, haptoglobin, haptoglobin-related protein, hemoglobin subunit alpha, hemoglobin subunit beta, hemoglobin subunit delta, and myoglobin.

As used herein, the term “globulin family” refers to any one or more of the following proteins: alpha-2 antiplasmin, murinoglobulin-2, vitamin K-dependent protein C, serum albumin, angiotensinogen, murinoglobulin-1, Ig kappa chain C, Igh-6 protein, alpha-2-macroglobulin, murinoglobulin-1, complement factor properdin, haptoglobin, beta-2-microglobulin, ceruloplasmin, serotransferrin, similar to immunoglobulin kappa-chain VK-1, serine (or cysteine) proteinase inhibitor clade A member 4, alpha-2-macroglobulin, IgG-2a protein, prothrombin, alpha-1-macroglobulin, serum albumin, thyroxine-binding globulin, immunoglobulin heavy chain variable region, corticosteroid-binding globulin, Ig heavy chain V region IR2, murinoglobulin-2, Ig gamma-2B chain C region, Igh-6 protein, Ig lambda-2 chain C region, Ig delta chain C region, Ig gamma-2C chain C region, Igh-6 protein, immunoglobulin J chain, Ig kappa chain V region S211, serum amyloid A-1 protein, serum amyloid A-2 protein, serum amyloid A-4 protein, and serum amyloid A protein.

As used herein, the term “kininogen family” refers to any one or more of the following proteins: kininogen, kininogen 1, and T-kininogen 2.

As used herein, the term “lysine and conjugates” refers to any one or more of the following molecules: 5-hydroxylysine, fructosyllysine, gamma-glutamyl-alpha-lysine, lysine, N6,N6,N6-trimethyllysine, N6-acetyllysine, N6-methyllysine, N,N,N-trimethyl-5-aminovalerate, and pipecolate.

As used herein, the term “proteasome complex family” refers to any one or more of the following molecules: proteasome subunit alpha type, proteasome subunit alpha type-7, proteasome subunit alpha type-1, proteasome subunit alpha type-2, proteasome subunit alpha type-3, proteasome subunit alpha type-4, proteasome subunit alpha type-6, proteasome subunit beta, proteasome subunit beta type, proteasome subunit beta type-1, proteasome subunit beta type-10, and proteasome subunit beta type-3.

As used herein, the term “serpins superfamily members” refers to any one or more of the following proteins: alpha-1-antiproteinase, heparin cofactor 2, plasma protease Cl inhibitor, protein Z-dependent protease inhibitor, serine (or cysteine) peptidase inhibitor clade B member 10, serine (or cysteine) peptidase inhibitor clade B member 6a, serine (or cysteine) peptidase inhibitor clade C member 1, serine protease inhibitor A3C, serine protease inhibitor A3F, serine protease inhibitor A3K, serine protease inhibitor A3L, serine protease inhibitor A3M, serine protease inhibitor A3N, serine protease inhibitor Kazal-type 3-like, serpin All, serpin family F member 2, and thyroxine-binding globulin.

As used herein, the term “valerate and conjugates” refers to any one or more of the following molecules: 2,3-dihydroxyisovalerate, 2-hydroxy-3-methylvalerate, 3-methyl-2-oxovalerate, alpha-hydroxyisovalerate, beta-hydroxyisovalerate, and N,N,N-trimethyl -5-aminovalerate.

In one embodiment, the molecular signature or profile of the silent phase of Alzheimer's disease, of grade SI, of grade S2 and/or of grade S3 comprises at least 1 biomarker selected from the group of biomarkers of Table IB. In one embodiment, the molecular signature or profile of the silent phase of Alzheimer's disease, of grade SI, of grade S2 and/or of grade S3 comprises 1 biomarker selected from the group of biomarkers of Table IB. In one embodiment, the molecular signature or profile of the silent phase of Alzheimer's disease, of grade SI, of grade S2 and/or of grade S3 consists of 1 biomarker selected from the group of biomarkers of Table IB.

TABLE 1B
BIOMARKERS OF THE SILENT PHASE OF ALZHEIMER'S DISEASE

| | |
|---|---|
| 14-3-3 protein beta/alpha | T-kininogen 2 |
| 14-3-3 protein epsilon | 5-hydroxylysine |
| 14-3-3 protein eta | Fructosyllysine |
| 14-3-3 protein gamma | Gamma-glutamyl-alpha-lysine |
| 14-3-3 protein theta | Lysine |
| 14-3-3 protein zeta/delta | N⁶,N⁶,N⁶-trimethyllysine |
| Apolipoprotein A-I | N⁶-acetyllysine |
| Apolipoprotein A-II | N⁶-methyllysine |
| Apolipoprotein A-IV | N,N,N-trimethyl-5-aminovalerate |
| Apolipoprotein B-100 | Pipecolate |
| Apolipoprotein C-I | Proteasome subunit alpha type |
| Apolipoprotein C-II (predicted) | Proteasome subunit alpha type-7 |
| Apolipoprotein C-III | Proteasome subunit alpha type-1 |
| Apolipoprotein C-IV | Proteasome subunit alpha type-2 |
| Apolipoprotein D | Proteasome subunit alpha type-3 |
| Apolipoprotein E | Proteasome subunit alpha type-4 |
| RaT apolipoprotein E protein | Proteasome subunit alpha type-6 |
| Apolipoprotein H (beta-2-glycoprotein I) | Proteasome subunit beta |
| Apolipoprotein M | Proteasome subunit beta type |
| Apolipoprotein N | Proteasome subunit beta type-1 |
| Actin-related protein 2 | Proteasome subunit beta type-10 |
| Actin-related protein 2/3 complex subunit | Proteasome subunit beta type-3 |
| 1B | |
| Actin-related protein 2/3 complex subunit | Alpha-1-antiproteinase |
| 3 | |
| Actin-related protein 2/3 complex subunit | Heparin cofactor 2 |
| 4 | |
| Actin-related protein 2/3 complex subunit | Plasma protease C1 inhibitor |
| 5 | |
| Actin-related protein 3 | Protein Z-dependent protease inhibitor |
| Arp2/3 complex 34 kDa subunit | Serine (or cysteine) peptidase inhibitor |
| | clade B member 10 |
| Carboxylesterase 1C | Serine (or cysteine) peptidase inhibitor |
| | clade B member 6a |
| Carboxylesterase 1E | Serine (or cysteine) peptidase inhibitor |
| | clade C member 1 |
| 2-methylbutyrylcarnitine (C5) | Serine protease inhibitor A3C |
| Acetylcarnitine (C2) | Serine protease inhibitor A3F |
| Arachidonoylcarnitine (C20:4) | Serine protease inhibitor A3K |
| Butyrylcarnitine (C4) | Serine protease inhibitor A3L |
| Carnitine | Serine protease inhibitor A3M |
| Cis-4-decenoylcarnitine (C10:1) | Serine protease inhibitor A3N |
| Isobutyrylcarnitine (C4) | Serine protease inhibitor Kazal-type 3-like |
| Isovalerylcarnitine (C5) | Serpin A11 |
| Laurylcarnitine (C12) | Serpin family F member 2 |
| Linoleoylcarnitine (C18:2) | Thyroxine-binding globulin |
| Myristoylcarnitine (C14) | 2,3-dihydroxyisovalerate |
| Octanoylcarnitine (C8) | 2-hydroxy-3-methylvalerate |
| Oleoylcarnitine (C18) | 3-methyl-2-oxovalerate |
| Palmitoleoylcarnitine (C16:1) | Alpha-hydroxyisovalerate |
| Palmitoylcarnitine (C16) | Beta-hydroxyisovalerate |
| Propionylcarnitine (C3) | N,N,N-trimethyl-5-aminovalerate |
| Stearoylcarnitine (C18) | 10 kDa heat shock protein, mitochondrial |
| (S)-3-hydroxybutyrylcarnitine | 1-methyladenosine |
| Deoxycarnitine | 1-methyl-5-imidazoleacetate |
| Beta-muricholate | 3,4-dihydroxybutyrate |
| Chenodeoxycholate | 3-amino-2-piperidone |
| Cholate | 3-methyl-2-oxobutyrate |
| Deoxycholate | 4-methyl-2-oxopentanoate |
| Glycocholate | 5-methyl-2′-deoxycytidine |
| Taurochenodeoxycholate | 5-methylthioadenosine (MTA) |
| Taurocholate | 5-oxoproline |
| Taurodeoxycholate | Adenylate kinase 4, mitochondrial |
| Coagulation factor V | Allantoic acid |
| Coagulation factor IX | Alpha-1B-glycoprotein |
| Coagulation factor VII | Alpha-actinin-1 |
| Coagulation factor X | Alpha-soluble NSF attachment protein |
| Coagulation factor XI | Anserine |
| Coagulation factor XII | Anti-F4/80 kappa light chain variable |
| | region |
| Coagulation factor XIII A chain | Arabonate/xylonate |
| Coagulation factor XIII B chain | Biotinidase |
| Complement factor B | BWK3 |
| Complement C1q subcomponent subunit | C4b-binding protein beta chain |
| A | |
| Complement C1q subcomponent subunit | Calpain small subunit 1 |
| B | |
| Complement C1q subcomponent subunit | Calreticulin |
| C | |
| Complement C1r subcomponent | Carboxypeptidase B2 |
| Complement C1r subcomponent-like | Carnosine |
| protein | |
| Complement C1s subcomponent | Chromogranin A |
| Complement C1s subcomponent | Clathrin heavy chain |
| Complement C2 | Coactosin-like protein |
| Complement C3 | Complement C6 |
| Complement C4 | Creatine |
| Complement C4A | Creatinine |
| Complement C4B | Cysteine-glutathione disulfide |
| C4B-binding protein alpha chain | Dimethylarginine |
| C4B-binding protein beta chain | Dimethyl sulfone |
| Complement C4-like | EGF-containing fibulin-like extracellular |
| | matrix protein 1 |
| Complement C5 | EH domain-containing protein 3 |
| Complement C7 | Elongation factor 1-alpha |
| Complement C8 alpha chain | Ergothioneine |
| Complement C8 beta chain | Erythronate |
| Complement C8 gamma chain | Ethylmalonate |
| Complement component C9 | Extracellular matrix protein 1 |
| Complement factor D | F-actin-capping protein subunit alpha-2 |
| Complement factor H | Fibronectin 1 |
| Complement factor H-related protein | Fibulin-1 |
| Complement factor H-related protein 1 | Fibulin-5 |
| Complement factor H-related protein 2 | Fructose |
| Complement factor H-related protein 3 | Fructose-bisphosphate aldolase |
| Complement factor H-related protein 4 | FYN-binding protein 1 |
| Complement factor I | Gelsolin |
| Creatine kinase B-type | Glucuronate |
| Creatine kinase M-type | Glucose |
| Globin a2 | Glycosylphosphatidylinositol specific |
| | phospholipase D1 |
| Globin a4 | HGF activator |
| Globin c2 | Histidine-rich glycoprotein |
| Globin c3 | Hyaluronidase |
| Globin d1 | Hydroxyproline |
| Haptoglobin | Ighg protein |
| Haptoglobin-related protein | Integrin beta |
| Hemoglobin subunit alpha | Integrin subunit alpha V |
| Hemoglobin subunit beta | Interleukin 1 receptor accessory protein |
| Hemoglobin subunit delta | Keratin, type II cytoskeletal 5 |
| Myoglobin | Lipase |
| Alpha-2 antiplasmin | Lumican |
| Murinoglobulin-2 | Lysyl oxidase-like 1 |
| Vitamin K-dependent protein C | Macrophage migration inhibitory factor |
| Serum albumin | Major urinary protein |
| Angiotensinogen | Malate dehydrogenase, cytoplasmic |
| Murinoglobulin-1 | Mannan-binding lectin serine peptidase 2 |
| Ig kappa chain C | Mannose-binding protein A |
| Igh-6 protein | Microfibril-associated glycoprotein 4 |
| Alpha-2-macroglobulin | Multiple inositol polyphosphate |
| | phosphatase 1 |
| Complement factor properdin | Myosin regulatory light polypeptide 9 |
| Haptoglobin | Myosin regulatory light chain RLC-A |
| Beta-2-microglobulin | N-acetylalanine |
| Ceruloplasmin | N-acetylasparagine |
| Serotransferrin | Octadecanedioate (C18) |
| Similar to immunoglobulin kappa-chain | Oleate/vaccenate (18:1) |
| VK-1 | |
| Serine (or cysteine) proteinase inhibitor | Palmitate (16:0) |
| clade A member 4 | |
| Alpha-2-muacroglobulin | Palmitoleate (16:1n7) |
| IgG-2a protein | Peptidyl-prolyl cis-trans isomerase A |
| Prothrombin | Peptidly-prolyl cis-trans isomerase |
| | FKBP1A |
| Alpha-1-macroglobulin | Peroxiredoxin 3 |
| IgG-2a protein | Peptidyl-prolyl cis-trans isomerase A |
| Prothrombin | Peptidyl-prolyl cis-trans isomerase |
| | FKBP1A |
| Alpha-1-macroglobulin | Peroxiredoxin 3 |
| Serum albumin | Phenylalanine |
| Thyroxine-binding globulin | Polyubiquitin-C |
| Immunoglobulin heavy chain variable | Prolylhydroxyproline |
| region | |
| Corticosteroid-binding globulin | Rat MHC class I truncated cell surface |
| | antigen mRNA |
| Ig heavy chain V region IR2 | Retinoic acid receptor responder 2 |
| Murinoglobulin-2 | Retinol-binding protein |
| Ig gamma-2B chain C region | Ribonate (ribonolactone) |
| | Ribulonate/xylulonate/lyxonate |
| Igh-6 protein | Sacsin molecular chaperone |
| Ig lambda-2 chain C region | Serum amyloid P-component |
| Ig delta chain C region | Sphingosine 1-phosphate |
| Ig gamma-2C chain C region | Sulfate |
| Igh-6 protein | Superoxide dismutase [Mn], |
| | mitochondrial |
| Immunoglobulin J chain | Talin 2 |
| Ig kappa chain V region S211 | Tartronate (hydroxymalonate) |
| Serum amyloid A-1 protein | Thioredoxin |
| Serum amyloid A-2 protein | Tmprss13 protein |
| Serum amyloid A-4 protein | Transferrin receptor protein 1 |
| Serum amyloid A protein | Iransthyretin |
| Kininogen | Urinary protein 1 |
| Kininogen 1 | Voltage-dependent anion-selective |
| | channel protein 3 |
| Carboxylesterase 1 | Xaa-Pro aminopeptidase 2 |

In one embodiment, the molecular signature or profile of the silent phase of Alzheimer's disease, of grade SI, of grade S2 and/or of grade S3 comprises at least 1 biomarker selected from the group of biomarkers of Table 2A. In one embodiment, the molecular signature or profile of the silent phase of Alzheimer's disease, of grade SI, of grade S2 and/or of grade S3 comprises 1 biomarker selected from the group of biomarkers of Table 2A. In one embodiment, the molecular signature or profile of the silent phase of Alzheimer's disease, of grade SI, of grade S2 and/or of grade S3 consists of 1 biomarker selected from the group of biomarkers of Table 2A.

TABLE 2A
BIOMARKERS OF THE SILENT PHASE
OF ALZHEIMER'S DISEASE

14-3-3 proteins
1-methyladenosine
3,4-dihydroxybutyrate
3-amino-2-piperidone
4-methyl-2-oxopentanoate
5-methylthioadenosine (MTA)
5-oxoproline
Alpha-1B-glycoprotein
Apolipoproteins
Arabonate/xylonate
Biotinidase
Carboxyesterase 1 family
Carboxypeptidase B2
Carnitine and conjugates
Cholate and conjugates
Coagulation factor family
Complement system family
Creatine
Creatinine
Creatinine kinase family
Cysteine-glutathione disulfide
Dimethyl sulfone
Dimethylarginine
EGF-containing fibulin-like extracellular matrix protein 1
Ergothioneine
Erythronate
Ethylmalonate
Extracellular matri|x protein 1
Fibulin-l
Gelsolin
Glucose
Globin family
Globulin family
Histidine-rich glycoprotein
Hydroxyproline
Kininogen family
Lumican
Lysine and conjugates
N-acetylalanine
Phenylalanine
Prolylhydroxyproline
Ribulonate/xylulonate/lyxonate
Serpin superfamily members
Serum amyloid P-component
Tartronate (hydroxymalonate)
Transthyretin
Valerate and conjugates

In one embodiment, the molecular signature or profile of the silent phase of Alzheimer's disease, of grade SI, of grade S2 and/or of grade S3 comprises at least 1 biomarker selected from the group of biomarkers of Table 2B. In one embodiment, the molecular signature or profile of the silent phase of Alzheimer's disease, of grade SI, of grade S2 and/or of grade S3 comprises 1 biomarker selected from the group of biomarkers of Table 2B. In one embodiment, the molecular signature or profile of the silent phase of Alzheimer's disease, of grade SI, of grade S2 and/or of grade S3 consists of 1 biomarker selected from the group of biomarkers of Table 2B.

TABLE 2B
BIOMARKERS OF THE SILENT PHASE
OF ALZHEIMER'S DISEASE

10 kDa heat shock protein, mitochondrial
14-3-3 proteins
1-methyl-5-imidazoleacetate
3-methyl-2-oxobutyrate
5-methyl-2′-deoxycytidine
Adenylate kinase 4, mitochondrial
Alpha-actinin-1
Alpha-soluble NSF attachment protein
Anserine
Anti-F4/80 kappa light chain variable region
Apolipoproteins
BWK3
Calpain small subunit 1
Carboxyesterase 1 family
Carnitine and conjugates
Carnosine
Cholate and conjugates
Coactosin-like protein
Coagulation factor family
Complement system family
EH domain-containing protein 3
Fibronectin 1
Fibulin-5
Fructose
FYN-binding protein 1
Globin family
Globulin family
Glucuronate
Integrin beta
Lipase
Lysine and conjugates
Lysyl oxidase-like 1
Malate dehydrogenase, cytoplasmic
Multiple inositol polyphosphate phosphatase 1
Oleate/vaccenate (18:1)
Palmitate (16:0)
Peptidyl-prolyl cis-trans isomerase A
Peroxiredoxin 3
Retinoic acid receptor responder 2
Ribonate (ribonolactone)
Sacsin molecular chaperone
Serpin superfamily members
Superoxide dismutase [Mn], mitochondrial
Thioredoxin
Tmprss13 protein
Urinary protein 1
Valerate and conjugates
Xaa-Pro aminopeptidase 2

In one embodiment, the molecular signature or profile of the silent phase of Alzheimer's disease, of grade SI, of grade S2 and/or of grade S3 comprises at least 1 biomarker selected from the group of biomarkers of Table 2C. In one embodiment, the molecular signature or profile of the silent phase of Alzheimer's disease, of grade SI, of grade S2 and/or of grade S3 comprises 1 biomarker selected from the group of biomarkers of Table 2C. In one embodiment, the molecular signature or profile of the silent phase of Alzheimer's disease, of grade SI, of grade S2 and/or of grade S3 consists of 1 biomarker selected from the group of biomarkers of Table 2C.

TABLE 2C
BIOMARKERS OF THE SILENT PHASE
OF ALZHEIMER'S DISEASE

14-3-3 proteins
Allantoic acid
Apolipoproteins
Arp2/3 complex proteins
Calreticulin
Carnitine and conjugates
Cholate and conjugates
Chromogranin A
Clathrin heavy chain
Coagulation factor family
Complement system family
Creatinine kinase family
Dimethylarginine
Elongation factor 1-alpha
F-actin-capping protein subunit alpha-2
Fibulin-5
Fructose-bisphosphate aldolase
Globin family
Globulin family
Glycosylphosphatidylinositol specific phospholipase D1
HGF activator
Hyaluronidase
Ighg protein
Integrin subunit alpha V
Interleukin 1 receptor accessory protein
Keratin, type II cytoskeletal 5
Lysine and conjugates
Macrophage migration inhibitory factor
Major urinary protein
Mannan-binding lectin serine peptidase 2
Mannose-binding protein A
Microfibril-associated glycoprotein 4
Myosin regulatory light chain RLC-A
Myosin regulatory light polypeptide 9
N-acetylasparagine
Octadecanedioate (C18)
Palmitoleate (16:1n7)
Peptidyl-prolyl cis-trans isomerase FKBP1A
Polyubiquitin-C
Proteasome complex family
Retinol-binding protein
Ribulonate/xylulonate/lyxonate
Sphingosine 1-phosphate
Sulfate
Talin 2
Tmprss13 protein
Transferrin receptor protein 1
Voltage-dependent anion-selective channel protein 3

In one embodiment, the molecular signature or profile of the silent phase of Alzheimer's disease, of grade SI, of grade S2 and/or of grade S3 comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or more biomarkers selected from the group of biomarkers of Table 1A or of Table IB. In one embodiment, the molecular signature or profile of the silent phase of Alzheimer's disease, of grade SI, of grade S2 and/or of grade S3 comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, or more biomarkers selected from the group of biomarkers of Table 1A or of Table IB.

In one embodiment, the molecular signature or profile of the silent phase of Alzheimer's disease, of grade SI, of grade S2 and/or of grade S3 consists of 2, 3, 4, 5, 6, 7, 8, 9, 10, 11, 12, 13, 14, 15, 16, 17, 18, 19, 20, 21, 22, 23, 24, 25, 26, 27, 28, 29, 30, or more biomarkers selected from the group of biomarkers of Table 1A or of Table IB.

In one embodiment, the molecular signature or profile of the silent phase of Alzheimer's disease, of grade SI, of grade S2 and/or of grade S3 comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or more biomarkers selected from the group of biomarkers of Table 2A, Table 2B or Table 2C. In one embodiment, the molecular signature or profile of the silent phase of Alzheimer's disease, of grade SI, of grade S2 and/or of grade S3 comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, or more biomarkers selected from the group of biomarkers of Table 2A, Table 2B or Table 2C. In one embodiment, the molecular signature or profile of the silent phase of Alzheimer's disease, of grade SI, of grade S2 and/or of grade S3 consists of 2, 3, 4, 5, 6, 7, 8, 9, 10, 11, 12, 13, 14, 15, 16, 17, 18, 19, 20, 21, 22, 23, 24, 25, 26, 27, 28, 29, 30, or more biomarkers selected from the group of biomarkers of Table 2A, Table 2B or Table 2C.

In one embodiment, the molecular signature or profile of the silent phase of Alzheimer's disease, of grade SI, of grade S2 and/or of grade S3 comprises at least 2 biomarkers selected from the group of biomarkers of Table 1A or of Table IB. In one embodiment, the molecular signature or profile of the silent phase of Alzheimer's disease, of grade SI, of grade S2 and/or of grade S3 comprises 2 biomarkers selected from the group of biomarkers of Table 1A or of Table IB. In one embodiment, the molecular signature or profile of the silent phase of Alzheimer's disease, of grade SI, of grade S2 and/or of grade S3 consists of 2 biomarkers selected from the group of biomarkers of Table 1A or of Table IB.

In one embodiment, the molecular signature or profile of the silent phase of Alzheimer's disease, of grade SI, of grade S2 and/or of grade S3 comprises at least 2 biomarkers selected from the group of biomarkers of Table 2A, Table 2B or Table 2C.

In one embodiment, the molecular signature or profile of the silent phase of Alzheimer's disease, of grade SI, of grade S2 and/or of grade S3 comprises 2 biomarkers selected from the group of biomarkers of Table 2A, Table 2B or Table 2C.

In one embodiment, the molecular signature or profile of the silent phase of Alzheimer's disease, of grade SI, of grade S2 and/or of grade S3 consists of 2 biomarkers selected from the group of biomarkers of Table 2A, Table 2B or Table 2C.

In one embodiment, the molecular signature or profile of the silent phase of Alzheimer's disease, of grade SI, of grade S2 and/or of grade S3 comprises at least 3 biomarkers selected from the group of biomarkers of Table 1A or of Table IB. In one embodiment, the molecular signature or profile of the silent phase of Alzheimer's disease, of grade SI, of grade S2 and/or of grade S3 comprises 3 biomarkers selected from the group of biomarkers of Table 1A or of Table IB. In one embodiment, the molecular signature or profile of the silent phase of Alzheimer's disease, of grade SI, of grade S2 and/or of grade S3 consists of 3 biomarkers selected from the group of biomarkers of Table 1A or of Table IB.

In one embodiment, the molecular signature or profile of the silent phase of Alzheimer's disease, of grade SI, of grade S2 and/or of grade S3 comprises at least 3 biomarkers selected from the group of biomarkers of Table 2A, Table 2B or Table 2C.

In one embodiment, the molecular signature or profile of the silent phase of Alzheimer's disease, of grade SI, of grade S2 and/or of grade S3 comprises 3 biomarkers selected from the group of biomarkers of Table 2A, Table 2B or Table 2C. In one embodiment, the molecular signature or profile of the silent phase of Alzheimer's disease, of grade SI, of grade S2 and/or of grade S3 consists of 3 biomarkers selected from the group of biomarkers of Table 2A, Table 2B or Table 2C.

In one embodiment, the molecular signature or profile of the silent phase of Alzheimer's disease, of grade SI, of grade S2 and/or of grade S3 comprises at least 4 biomarkers selected from the group of biomarkers of Table 1A or of Table IB. In one embodiment, the molecular signature or profile of the silent phase of Alzheimer's disease, of grade SI, of grade S2 and/or of grade S3 comprises 4 biomarkers selected from the group of biomarkers of Table 1A or of Table IB. In one embodiment, the molecular signature or profile of the silent phase of Alzheimer's disease, of grade SI, of grade S2 and/or of grade S3 consists of 4 biomarkers selected from the group of biomarkers of Table 1A or of Table IB.

In one embodiment, the molecular signature or profile of the silent phase of Alzheimer's disease, of grade SI, of grade S2 and/or of grade S3 comprises at least 4 biomarkers selected from the group of biomarkers of Table 2A, Table 2B or Table 2C.

In one embodiment, the molecular signature or profile of the silent phase of Alzheimer's disease, of grade SI, of grade S2 and/or of grade S3 comprises 4 biomarkers selected from the group of biomarkers of Table 2A, Table 2B or Table 2C. In one embodiment, the molecular signature or profile of the silent phase of Alzheimer's disease, of grade SI, of grade S2 and/or of grade S3 consists of 4 biomarkers selected from the group of biomarkers of Table 2A, Table 2B or Table 2C.

In one embodiment, the molecular signature or profile of the silent phase of Alzheimer's disease, of grade SI, of grade S2 and/or of grade S3 comprises at least 5 biomarkers selected from the group of biomarkers of Table 1A or of Table IB. In one embodiment, the molecular signature or profile of the silent phase of Alzheimer's disease, of grade SI, of grade S2 and/or of grade S3 comprises 5 biomarkers selected from the group of biomarkers of Table 1A or of Table IB. In one embodiment, the molecular signature or profile of the silent phase of Alzheimer's disease, of grade SI, of grade S2 and/or of grade S3 consists of 5 biomarkers selected from the group of biomarkers of Table 1A or of Table IB.

In one embodiment, the molecular signature or profile of the silent phase of Alzheimer's disease, of grade SI, of grade S2 and/or of grade S3 comprises at least 5 biomarkers selected from the group of biomarkers of Table 2A, Table 2B or Table 2C.

In one embodiment, the molecular signature or profile of the silent phase of Alzheimer's disease, of grade SI, of grade S2 and/or of grade S3 comprises 5 biomarkers selected from the group of biomarkers of Table 2A, Table 2B or Table 2C. In one embodiment, the molecular signature or profile of the silent phase of Alzheimer's disease, of grade SI, of grade S2 and/or of grade S3 consists of 5 biomarkers selected from the group of biomarkers of Table 2A, Table 2B or Table 2C.

In one exemplary and non-limiting embodiment, the molecular signature or profile of the silent phase of Alzheimer's disease, of grade SI, of grade S2 and/or of grade S3 comprises at least 5 biomarkers selected from, or consists of the 5 following biomarkers: fructosyllysine, integrin beta, isobutyrylcamitine (C4), myosin regulatory light chain RLC-A and talin 2.

In one embodiment, the molecular signature or profile of the silent phase of Alzheimer's disease, of grade SI, of grade S2 and/or of grade S3 comprises at least 6 biomarkers selected from the group of biomarkers of Table 1A or of Table IB. In one embodiment, the molecular signature or profile of the silent phase of Alzheimer's disease, of grade SI, of grade S2 and/or of grade S3 comprises 6 biomarkers selected from the group of biomarkers of Table 1A or of Table IB. In one embodiment, the molecular signature or profile of the silent phase of Alzheimer's disease, of grade SI, of grade S2 and/or of grade S3 consists of 6 biomarkers selected from the group of biomarkers of Table 1A or of Table IB.

In one embodiment, the molecular signature or profile of the silent phase of Alzheimer's disease, of grade SI, of grade S2 and/or of grade S3 comprises at least 6 biomarkers selected from the group of biomarkers of Table 2A, Table 2B or Table 2C.

In one embodiment, the molecular signature or profile of the silent phase of Alzheimer's disease, of grade SI, of grade S2 and/or of grade S3 comprises 6 biomarkers selected from the group of biomarkers of Table 2A, Table 2B or Table 2C. In one embodiment, the molecular signature or profile of the silent phase of Alzheimer's disease, of grade SI, of grade S2 and/or of grade S3 consists of 6 biomarkers selected from the group of biomarkers of Table 2A, Table 2B or Table 2C.

In one exemplary and non-limiting embodiment, the molecular signature or profile of the silent phase of Alzheimer's disease, of grade SI, of grade S2 and/or of grade S3 comprises at least 6 biomarkers selected from, or consists of the 6 following biomarkers: fructosyllysine, Igh-6 protein, myosin regulatory light chain RLC-A, octadecanedioate (C18), ribonate (ribonolactone) and talin 2.

In one embodiment, the molecular signature or profile of the silent phase of Alzheimer's disease, of grade SI, of grade S2 and/or of grade S3 comprises at least 7 biomarkers selected from the group of biomarkers of Table 1A or of Table IB. In one embodiment, the molecular signature or profile of the silent phase of Alzheimer's disease, of grade SI, of grade S2 and/or of grade S3 comprises 7 biomarkers selected from the group of biomarkers of Table 1A or of Table IB. In one embodiment, the molecular signature or profile of the silent phase of Alzheimer's disease, of grade SI, of grade S2 and/or of grade S3 consists of 7 biomarkers selected from the group of biomarkers of Table 1A or of Table IB.

In one embodiment, the molecular signature or profile of the silent phase of Alzheimer's disease, of grade SI, of grade S2 and/or of grade S3 comprises at least 7 biomarkers selected from the group of biomarkers of Table 2A, Table 2B or Table 2C.

In one embodiment, the molecular signature or profile of the silent phase of Alzheimer's disease, of grade SI, of grade S2 and/or of grade S3 comprises 7 biomarkers selected from the group of biomarkers of Table 2A, Table 2B or Table 2C. In one embodiment, the molecular signature or profile of the silent phase of Alzheimer's disease, of grade SI, of grade S2 and/or of grade S3 consists of 7 biomarkers selected from the group of biomarkers of Table 2A, Table 2B or Table 2C.

In one embodiment, the molecular signature or profile of the silent phase of Alzheimer's disease, of grade SI, of grade S2 and/or of grade S3 comprises at least 8 biomarkers selected from the group of biomarkers of Table 1A or of Table IB. In one embodiment, the molecular signature or profile of the silent phase of Alzheimer's disease, of grade SI, of grade S2 and/or of grade S3 comprises 8 biomarkers selected from the group of biomarkers of Table 1A or of Table IB. In one embodiment, the molecular signature or profile of the silent phase of Alzheimer's disease, of grade SI, of grade S2 and/or of grade S3 consists of 8 biomarkers selected from the group of biomarkers of Table 1A or of Table IB.

In one embodiment, the molecular signature or profile of the silent phase of Alzheimer's disease, of grade SI, of grade S2 and/or of grade S3 comprises at least 8 biomarkers selected from the group of biomarkers of Table 2A, Table 2B or Table 2C.

In one embodiment, the molecular signature or profile of the silent phase of Alzheimer's disease, of grade SI, of grade S2 and/or of grade S3 comprises 8 biomarkers selected from the group of biomarkers of Table 2A, Table 2B or Table 2C. In one embodiment, the molecular signature or profile of the silent phase of Alzheimer's disease, of grade SI, of grade S2 and/or of grade S3 consists of 8 biomarkers selected from the group of biomarkers of Table 2A, Table 2B or Table 2C.

In one embodiment, the molecular signature or profile of the silent phase of Alzheimer's disease, of grade SI, of grade S2 and/or of grade S3 comprises at least 9 biomarkers selected from the group of biomarkers of Table 1A or of Table IB. In one embodiment, the molecular signature or profile of the silent phase of Alzheimer's disease, of grade SI, of grade S2 and/or of grade S3 comprises 9 biomarkers selected from the group of biomarkers of Table 1A or of Table IB. In one embodiment, the molecular signature or profile of the silent phase of Alzheimer's disease, of grade SI, of grade S2 and/or of grade S3 consists of 9 biomarkers selected from the group of biomarkers of Table 1A or of Table IB

In one embodiment, the molecular signature or profile of the silent phase of. Alzheimer's disease, of grade SI, of grade S2 and/or of grade S3 comprises at least 9 biomarkers selected from the group of biomarkers of Table 2A, Table 2B or Table 2C.

In one embodiment, the molecular signature or profile of the silent phase of Alzheimer's disease, of grade SI, of grade S2 and/or of grade S3 comprises 9 biomarkers selected from the group of biomarkers of Table 2A, Table 2B or Table 2C. In one embodiment, the molecular signature or profile of the silent phase of Alzheimer's disease, of grade SI, of grade S2 and/or of grade S3 consists of 9 biomarkers selected from the group of biomarkers of Table 2A, Table 2B or Table 2C.

In one embodiment, the molecular signature or profile of the silent phase of Alzheimer's disease, of grade SI, of grade S2 and/or of grade S3 comprises at least 10 biomarkers selected from the group of biomarkers of Table 1A or of Table IB. In one embodiment, the molecular signature or profile of the silent phase of Alzheimer's disease, of grade SI, of grade S2 and/or of grade S3 comprises 10 biomarkers selected from the group of biomarkers of Table 1A or of Table IB. In one embodiment, the molecular signature or profile of the silent phase of Alzheimer's disease, of grade SI, of grade S2 and/or of grade S3 consists of 10 biomarkers selected from the group of biomarkers of Table 1A or of Table IB.

In one embodiment, the molecular signature or profile of the silent phase of Alzheimer's disease, of grade SI, of grade S2 and/or of grade S3 comprises at least 10 biomarkers selected from the group of biomarkers of Table 2A, Table 2B or Table 2C. In one embodiment, the molecular signature or profile of the silent phase of Alzheimer's disease, of grade SI, of grade S2 and/or of grade S3 comprises 10 biomarkers selected from the group of biomarkers of Table 2A, Table 2B or Table 2C. In one embodiment, the molecular signature or profile of the silent phase of Alzheimer's disease, of grade SI, of grade S2 and/or of grade S3 consists of 10 biomarkers selected from the group of biomarkers of Table 2A, Table 2B or Table 2C.

In one embodiment, the molecular signature or profile of the silent phase of Alzheimer's disease, of grade SI, of grade S2 and/or of grade S3 comprises at least 11 biomarkers selected from the group of biomarkers of Table 1A or of Table IB. In one embodiment, the molecular signature or profile of the silent phase of Alzheimer's disease, of grade SI, of grade S2 and/or of grade S3 comprises 11 biomarkers selected from the group of biomarkers of Table 1A or of Table IB. In one embodiment, the molecular signature or profile of the silent phase of Alzheimer's disease, of grade SI, of grade S2 and/or of grade S3 consists of 11 biomarkers selected from the group of biomarkers of Table 1A or of Table IB.

In one embodiment, the molecular signature or profile of the silent phase of Alzheimer's disease, of grade SI, of grade S2 and/or of grade S3 comprises at least 11 biomarkers selected from the group of biomarkers of Table 2A, Table 2B or Table 2C. In one embodiment, the molecular signature or profile of the silent phase of Alzheimer's disease, of grade SI, of grade S2 and/or of grade S3 comprises 11 biomarkers selected from the group of biomarkers of Table 2A, Table 2B or Table 2C. In one embodiment, the molecular signature or profile of the silent phase of Alzheimer's disease, of grade SI, of grade S2 and/or of grade S3 consists of 11 biomarkers selected from the group of biomarkers of Table 2A, Table 2B or Table 2C.

In one embodiment, the molecular signature or profile of the silent phase of Alzheimer's disease, of grade SI, of grade S2 and/or of grade S3 comprises at least 12 biomarkers selected from the group of biomarkers of Table 1A or of Table IB. In one embodiment, the molecular signature or profile of the silent phase of Alzheimer's disease, of grade SI, of grade S2 and/or of grade S3 comprises 12 biomarkers selected from the group of biomarkers of Table 1A or of Table IB. In one embodiment, the molecular signature or profile of the silent phase of Alzheimer's disease, of grade SI, of grade S2 and/or of grade S3 consists of 12 biomarkers selected from the group of biomarkers of Table 1A or of Table IB.

In one embodiment, the molecular signature or profile of the silent phase of Alzheimer's disease, of grade SI, of grade S2 and/or of grade S3 comprises at least 12 biomarkers selected from the group of biomarkers of Table 2A, Table 2B or Table 2C. In one embodiment, the molecular signature or profile of the silent phase of Alzheimer's disease, of grade SI, of grade S2 and/or of grade S3 comprises 12 biomarkers selected from the group of biomarkers of Table 2A, Table 2B or Table 2C. In one embodiment, the molecular signature or profile of the silent phase of Alzheimer's disease, of grade SI, of grade S2 and/or of grade S3 consists of 12 biomarkers selected from the group of biomarkers of Table 2A, Table 2B or Table 2C.

In one embodiment, the molecular signature or profile of the silent phase of Alzheimer's disease, of grade SI, of grade S2 and/or of grade S3 comprises at least 13 biomarkers selected from the group of biomarkers of Table 1A or of Table IB. In one embodiment, the molecular signature or profile of the silent phase of Alzheimer's disease, of grade SI, of grade S2 and/or of grade S3 comprises 13 biomarkers selected from the group of biomarkers of Table 1A or of Table IB. In one embodiment, the molecular signature or profile of the silent phase of Alzheimer's disease, of grade SI, of grade S2 and/or of grade S3 consists of 13 biomarkers selected from the group of biomarkers of Table 1A or of Table IB.

In one embodiment, the molecular signature or profile of the silent phase of Alzheimer's disease, of grade SI, of grade S2 and/or of grade S3 comprises at least 13 biomarkers selected from the group of biomarkers of Table 2A, Table 2B or Table 2C. In one embodiment, the molecular signature or profile of the silent phase of Alzheimer's disease, of grade SI, of grade S2 and/or of grade S3 comprises 13 biomarkers selected from the group of biomarkers of Table 2A, Table 2B or Table 2C. In one embodiment, the molecular signature or profile of the silent phase of Alzheimer's disease, of grade SI, of grade S2 and/or of grade S3 consists of 13 biomarkers selected from the group of biomarkers of Table 2A, Table 2B or Table 2C.

In one embodiment, the molecular signature or profile of the silent phase of Alzheimer's disease, of grade SI, of grade S2 and/or of grade S3 comprises at least 14 biomarkers selected from the group of biomarkers of Table 1A or of Table IB. In one embodiment, the molecular signature or profile of the silent phase of Alzheimer's disease, of grade SI, of grade S2 and/or of grade S3 comprises 14 biomarkers selected from the group of biomarkers of Table 1A or of Table IB. In one embodiment, the molecular signature or profile of the silent phase of Alzheimer's disease, of grade SI, of grade S2 and/or of grade S3 consists of 14 biomarkers selected from the group of biomarkers of Table 1A or of Table IB.

In one embodiment, the molecular signature or profile of the silent phase of Alzheimer's disease, of grade SI, of grade S2 and/or of grade S3 comprises at least 14 biomarkers selected from the group of biomarkers of Table 2A, Table 2B or Table 2C. In one embodiment, the molecular signature or profile of the silent phase of Alzheimer's disease, of grade SI, of grade S2 and/or of grade S3 comprises 14 biomarkers selected from the group of biomarkers of Table 2A, Table 2B or Table 2C. In one embodiment, the molecular signature or profile of the silent phase of Alzheimer's disease, of grade SI, of grade S2 and/or of grade S3 consists of 14 biomarkers selected from the group of biomarkers of Table 2A, Table 2B or Table 2C.

In one exemplary and non-limiting embodiment, the molecular signature or profile of the silent phase of Alzheimer's disease, of grade SI, of grade S2 and/or of grade S3 comprises at least 14 biomarkers selected from, or consists of the 14 following biomarkers: 10 kDa heat shock protein, mitochondrial; 5-hydroxylysine; adenylate kinase 4, mitochondrial; calreticulin; creatine kinase B-type; ergothioneine; peptidyl-prolyl cis-transisomerase FKBP1A; fructosyllysine; globin c2; integrin subunit alpha V; myoglobin; retinoic acid receptor responder 2; Tmprss13 protein; and transferrin receptor protein 1.

In one embodiment, the molecular signature or profile of the silent phase of Alzheimer's disease, of grade SI, of grade S2 and/or of grade S3 comprises at least 15 biomarkers selected from the group of biomarkers of Table 1A or of Table IB. In one embodiment, the molecular signature or profile of the silent phase of Alzheimer's disease, of grade SI, of grade S2 and/or of grade S3 comprises 15 biomarkers selected from the group of biomarkers of Table 1A or of Table IB. In one embodiment, the molecular signature or profile of the silent phase of Alzheimer's disease, of grade SI, of grade S2 and/or of grade S3 consists of 15 biomarkers selected from the group of biomarkers of Table 1A or of Table IB.

In one embodiment, the molecular signature or profile of the silent phase of Alzheimer's disease, of grade SI, of grade S2 and/or of grade S3 comprises at least 15 biomarkers selected from the group of biomarkers of Table 2A, Table 2B or Table 2C. In one embodiment, the molecular signature or profile of the silent phase of Alzheimer's disease, of grade SI, of grade S2 and/or of grade S3 comprises 15 biomarkers selected from the group of biomarkers of Table 2A, Table 2B or Table 2C. In one embodiment, the molecular signature or profile of the silent phase of Alzheimer's disease, of grade SI, of grade S2 and/or of grade S3 consists of 15 biomarkers selected from the group of biomarkers of Table 2A, Table 2B or Table 2C.

In one embodiment, the molecular signature or profile of the silent phase of Alzheimer's disease, of grade SI, of grade S2 and/or of grade S3 comprises at least 16 biomarkers selected from the group of biomarkers of Table 1A or of Table IB. In one embodiment, the molecular signature or profile of the silent phase of Alzheimer's disease, of grade SI, of grade S2 and/or of grade S3 comprises 16 biomarkers selected from the group of biomarkers of Table 1A or of Table IB. In one embodiment, the molecular signature or profile of the silent phase of Alzheimer's disease, of grade SI, of grade S2 and/or of grade S3 consists of 16 biomarkers selected from the group of biomarkers of Table 1A or of Table IB.

In one embodiment, the molecular signature or profile of the silent phase of Alzheimer's disease, of grade SI, of grade S2 and/or of grade S3 comprises at least 16 biomarkers selected from the group of biomarkers of Table 2A, Table 2B or Table 2C. In one embodiment, the molecular signature or profile of the silent phase of Alzheimer's disease, of grade SI, of grade S2 and/or of grade S3 comprises 16 biomarkers selected from the group of biomarkers of Table 2A, Table 2B or Table 2C. In one embodiment, the molecular signature or profile of the silent phase of Alzheimer's disease, of grade SI, of grade S2 and/or of grade S3 consists of 16 biomarkers selected from the group of biomarkers of Table 2A, Table 2B or Table 2C.

In one embodiment, the molecular signature or profile of the silent phase of Alzheimer's disease, of grade SI, of grade S2 and/or of grade S3 comprises at least 17 biomarkers selected from the group of biomarkers of Table 1A or of Table IB. In one embodiment, the molecular signature or profile of the silent phase of Alzheimer's disease, of grade SI, of grade S2 and/or of grade S3 comprises 17 biomarkers selected from the group of biomarkers of Table 1A or of Table IB. In one embodiment, the molecular signature or profile of the silent phase of Alzheimer's disease, of grade SI, of grade S2 and/or of grade S3 consists of 17 biomarkers selected from the group of biomarkers of Table 1A or of Table IB.

In one embodiment, the molecular signature or profile of the silent phase of Alzheimer's disease, of grade SI, of grade S2 and/or of grade S3 comprises at least 17 biomarkers selected from the group of biomarkers of Table 2A, Table 2B or Table 2C. In one embodiment, the molecular signature or profile of the silent phase of Alzheimer's disease, of grade SI, of grade S2 and/or of grade S3 comprises 17 biomarkers selected from the group of biomarkers of Table 2A, Table 2B or Table 2C. In one embodiment, the molecular signature or profile of the silent phase of Alzheimer's disease, of grade SI, of grade S2 and/or of grade S3 consists of 17 biomarkers selected from the group of biomarkers of Table 2A, Table 2B or Table 2C.

In one embodiment, the molecular signature or profile of the silent phase of Alzheimer's disease, of grade SI, of grade S2 and/or of grade S3 comprises at least 18 biomarkers selected from the group of biomarkers of Table 1A or of Table IB. In one embodiment, the molecular signature or profile of the silent phase of Alzheimer's disease, of grade SI, of grade S2 and/or of grade S3 comprises 18 biomarkers selected from the group of biomarkers of Table 1A or of Table IB. In one embodiment, the molecular signature or profile of the silent phase of Alzheimer's disease, of grade SI, of grade S2 and/or of grade S3 consists of 18 biomarkers selected from the group of biomarkers of Table 1A or of Table IB.

In one embodiment, the molecular signature or profile of the silent phase of Alzheimer's disease, of grade SI, of grade S2 and/or of grade S3 comprises at least 18 biomarkers selected from the group of biomarkers of Table 2A, Table 2B or Table 2C. In one embodiment, the molecular signature or profile of the silent phase of Alzheimer's disease, of grade SI, of grade S2 and/or of grade S3 comprises 18 biomarkers selected from the group of biomarkers of Table 2A, Table 2B or Table 2C. In one embodiment, the molecular signature or profile of the silent phase of Alzheimer's disease, of grade SI, of grade S2 and/or of grade S3 consists of 18 biomarkers selected from the group of biomarkers of Table 2A, Table 2B or Table 2C.

In one embodiment, the molecular signature or profile of the silent phase of Alzheimer's disease, of grade SI, of grade S2 and/or of grade S3 comprises at least 19 biomarkers selected from the group of biomarkers of Table 1A or of Table IB. In one embodiment, the molecular signature or profile of the silent phase of Alzheimer's disease, of grade SI, of grade S2 and/or of grade S3 comprises 19 biomarkers selected from the group of biomarkers of Table 1A or of Table IB. In one embodiment, the molecular signature or profile of the silent phase of Alzheimer's disease, of grade SI, of grade S2 and/or of grade S3 consists of 19 biomarkers selected from the group of biomarkers of Table 1A or of Table IB.

In one embodiment, the molecular signature or profile of the silent phase of Alzheimer's disease, of grade SI, of grade S2 and/or of grade S3 comprises at least 19 biomarkers selected from the group of biomarkers of Table 2A, Table 2B or Table 2C. In one embodiment, the molecular signature or profile of the silent phase of Alzheimer's disease, of grade SI, of grade S2 and/or of grade S3 comprises 19 biomarkers selected from the group of biomarkers of Table 2A, Table 2B or Table 2C. In one embodiment, the molecular signature or profile of the silent phase of Alzheimer's disease, of grade SI, of grade S2 and/or of grade S3 consists of 19 biomarkers selected from the group of biomarkers of Table 2A, Table 2B or Table 2C.

In one embodiment, the molecular signature or profile of the silent phase of Alzheimer's disease, of grade SI, of grade S2 and/or of grade S3 comprises at least 20 biomarkers selected from the group of biomarkers of Table 1A or of Table IB. In one embodiment, the molecular signature or profile of the silent phase of Alzheimer's disease, of grade SI, of grade S2 and/or of grade S3 comprises 20 biomarkers selected from the group of biomarkers of Table 1A or of Table IB. In one embodiment, the molecular signature or profile of the silent phase of Alzheimer's disease, of grade SI, of grade S2 and/or of grade S3 consists of 20 biomarkers selected from the group of biomarkers of Table 1A or of Table IB.

In one embodiment, the molecular signature or profile of the silent phase of Alzheimer's disease, of grade SI, of grade S2 and/or of grade S3 comprises at least 20 biomarkers selected from the group of biomarkers of Table 2A, Table 2B or Table 2C. In one embodiment, the molecular signature or profile of the silent phase of Alzheimer's disease, of grade SI, of grade S2 and/or of grade S3 comprises 20 biomarkers selected from the group of biomarkers of Table 2A, Table 2B or Table 2C. In one embodiment, the molecular signature or profile of the silent phase of Alzheimer's disease, of grade SI, of grade S2 and/or of grade S3 consists of 20 biomarkers selected from the group of biomarkers of Table 2A, Table 2B or Table 2C.

In one embodiment, the molecular signature or profile of the silent phase of Alzheimer's disease, of grade SI, of grade S2 and/or of grade S3 comprises at least 21 biomarkers selected from the group of biomarkers of Table 1A or of Table IB. In one embodiment, the molecular signature or profile of the silent phase of Alzheimer's disease, of grade SI, of grade S2 and/or of grade S3 comprises 21 biomarkers selected from the group of biomarkers of Table 1A or of Table IB. In one embodiment, the molecular signature or profile of the silent phase of Alzheimer's disease, of grade SI, of grade S2 and/or of grade S3 consists of 21 biomarkers selected from the group of biomarkers of Table 1A or of Table IB.

In one embodiment, the molecular signature or profile of the silent phase of Alzheimer's disease, of grade SI, of grade S2 and/or of grade S3 comprises at least 21 biomarkers selected from the group of biomarkers of Table 2A, Table 2B or Table 2C. In one embodiment, the molecular signature or profile of the silent phase of Alzheimer's disease, of grade SI, of grade S2 and/or of grade S3 comprises 21 biomarkers selected from the group of biomarkers of Table 2A, Table 2B or Table 2C. In one embodiment, the molecular signature or profile of the silent phase of Alzheimer's disease, of grade SI, of grade S2 and/or of grade S3 consists of 21 biomarkers selected from the group of biomarkers of Table 2A, Table 2B or Table 2C.

In one embodiment, the molecular signature or profile of the silent phase of Alzheimer's disease, of grade SI, of grade S2 and/or of grade S3 comprises at least 22 biomarkers selected from the group of Nebiomarkers of Table 1A or of Table IB. In one embodiment, the molecular signature or profile of the silent phase of Alzheimer's disease, of grade SI, of grade S2 and/or of grade S3 comprises 22 biomarkers selected from the group of biomarkers of Table 1A or of Table IB. In one embodiment, the molecular signature or profile of the silent phase of Alzheimer's disease, of grade SI, of grade S2 and/or of grade S3 consists of 22 biomarkers selected from the group of biomarkers of Table 1A or of Table IB.

In one embodiment, the molecular signature or profile of the silent phase of Alzheimer's disease, of grade SI, of grade S2 and/or of grade S3 comprises at least 22 biomarkers selected from the group of biomarkers of Table 2A, Table 2B or Table 2C. In one embodiment, the molecular signature or profile of the silent phase of Alzheimer's disease, of grade SI, of grade S2 and/or of grade S3 comprises 22 biomarkers selected from the group of biomarkers of Table 2A, Table 2B or Table 2C. In one embodiment, the molecular signature or profile of the silent phase of Alzheimer's disease, of grade SI, of grade S2 and/or of grade S3 consists of 22 biomarkers selected from the group of biomarkers of Table 2A, Table 2B or Table 2C.

In one embodiment, the molecular signature or profile of the silent phase of Alzheimer's disease, of grade SI, of grade S2 and/or of grade S3 comprises at least 23 biomarkers selected from the group of biomarkers of Table 1A or of Table IB. In one embodiment, the molecular signature or profile of the silent phase of Alzheimer's disease, of grade SI, of grade S2 and/or of grade S3 comprises 23 biomarkers selected from the group of biomarkers of Table 1A or of Table IB. In one embodiment, the molecular signature or profile of the silent phase of Alzheimer's disease, of grade SI, of grade S2 and/or of grade S3 consists of 23 biomarkers selected from the group of biomarkers of Table 1A or of Table IB.

In one embodiment, the molecular signature or profile of the silent phase of Alzheimer's disease, of grade SI, of grade S2 and/or of grade S3 comprises at least

23 biomarkers selected from the group of biomarkers of Table 2A, Table 2B or Table 2C. In one embodiment, the molecular signature or profile of the silent phase of Alzheimer's disease, of grade SI, of grade S2 and/or of grade S3 comprises 23 biomarkers selected from the group of biomarkers of Table 2A, Table 2B or Table 2C. In one embodiment, the molecular signature or profile of the silent phase of Alzheimer's disease, of grade SI, of grade S2 and/or of grade S3 consists of 23 biomarkers selected from the group of biomarkers of Table 2A, Table 2B or Table 2C.

In one embodiment, the molecular signature or profile of the silent phase of Alzheimer's disease, of grade SI, of grade S2 and/or of grade S3 comprises at least 24 biomarkers selected from the group of biomarkers of Table 1A or of Table IB. In one embodiment, the molecular signature or profile of the silent phase of Alzheimer's disease, of grade SI, of grade S2 and/or of grade S3 comprises 24 biomarkers selected from the group of biomarkers of Table 1A or of Table IB. In one embodiment, the molecular signature or profile of the silent phase of Alzheimer's disease, of grade SI, of grade S2 and/or of grade S3 consists of 24 biomarkers selected from the group of biomarkers of Table 1A or of Table IB.

In one embodiment, the molecular signature or profile of the silent phase of Alzheimer's disease, of grade SI, of grade S2 and/or of grade S3 comprises at least 24 biomarkers selected from the group of biomarkers of Table 2A, Table 2B or Table 2C.

In one embodiment, the molecular signature or profile of the silent phase of Alzheimer's disease, of grade SI, of grade S2 and/or of grade S3 comprises 24 biomarkers selected from the group of biomarkers of Table 2A, Table 2B or Table 2C. In one embodiment, the molecular signature or profile of the silent phase of Alzheimer's disease, of grade SI, of grade S2 and/or of grade S3 consists of 24 biomarkers selected from the group of biomarkers of Table 2A, Table 2B or Table 2C.

In one embodiment, the molecular signature or profile of the silent phase of Alzheimer's disease, of grade SI, of grade S2 and/or of grade S3 comprises at least 25 biomarkers selected from the group of biomarkers of Table 1A or of Table IB. In one embodiment, the molecular signature or profile of the silent phase of Alzheimer's disease, of grade SI, of grade S2 and/or of grade S3 comprises 25 biomarkers selected from the group of biomarkers of Table 1A or of Table IB. In one embodiment, the molecular signature or profile of the silent phase of Alzheimer's disease, of grade SI, of grade S2 and/or of grade S3 consists of 25 biomarkers selected from the group of biomarkers of Table 1A or of Table IB.

In one embodiment, the molecular signature or profile of the silent phase of Alzheimer's disease, of grade SI, of grade S2 and/or of grade S3 comprises at least 25 biomarkers selected from the group of biomarkers of Table 2A, Table 2B or Table 2C. In one embodiment, the molecular signature or profile of the silent phase of Alzheimer's disease, of grade SI, of grade S2 and/or of grade S3 comprises 25 biomarkers selected from the group of biomarkers of Table 2A, Table 2B or Table 2C. In one embodiment, the molecular signature or profile of the silent phase of Alzheimer's disease, of grade SI, of grade S2 and/or of grade S3 consists of 25 biomarkers selected from the group of biomarkers of Table 2A, Table 2B or Table 2C.

In one embodiment, the molecular signature or profile of the silent phase of Alzheimer's disease, of grade SI, of grade S2 and/or of grade S3 comprises at least 26 biomarkers selected from the group of biomarkers of Table 1A or of Table IB. In one embodiment, the molecular signature or profile of the silent phase of Alzheimer's disease, of grade SI, of grade S2 and/or of grade S3 comprises 26 biomarkers selected from the group of biomarkers of Table 1A or of Table IB. In one embodiment, the molecular signature or profile of the silent phase of Alzheimer's disease, of grade SI, of grade S2 and/or of grade S3 consists of 26 biomarkers selected from the group of biomarkers of Table 1A or of Table IB.

In one embodiment, the molecular signature or profile of the silent phase of Alzheimer's disease, of grade SI, of grade S2 and/or of grade S3 comprises at least 26 biomarkers selected from the group of biomarkers of Table 2A, Table 2B or Table 2C. In one embodiment, the molecular signature or profile of the silent phase of Alzheimer's disease, of grade SI, of grade S2 and/or of grade S3 comprises 26 biomarkers selected from the group of biomarkers of Table 2A, Table 2B or Table 2C. In one embodiment, the molecular signature or profile of the silent phase of Alzheimer's disease, of grade SI, of grade S2 and/or of grade S3 consists of 26 biomarkers selected from the group of biomarkers of Table 2A, Table 2B or Table 2C.

In one exemplary and non-limiting embodiment, the molecular signature or profile of the silent phase of Alzheimer's disease, of grade SI, of grade S2 and/or of grade S3 comprises at least 26 biomarkers selected from, or consists of the 26 following biomarkers: rat apolipoprotein E protein; Arp2/3 complex 34 kDa subunit; carnitine; isobutyrylcamitine (C4); isovalerylcarnitine (C5); coagulation factor VII; serine (or cysteine) proteinase inhibitor clade A member 4; Igh-6 protein; serum amyloid P-component; allantoic acid; calpain small subunit 1; carboxypeptidase B2; carnosine; clathrin heavy chain; complement C6; extracellular matrix protein 1; fructose-bisphosphate aldolase; keratin type II cytoskel et al 5; mannose-binding protein A; myosin regulatory light chain RLC-A; N-acetylasparagine; octadecanedioate (C18); ribonate (ribonolactone); ribulonate; talin 2; and Xaa-Pro aminopeptidase 2.

In one embodiment, the molecular signature or profile of the silent phase of Alzheimer's disease, of grade SI, of grade S2 and/or of grade S3 comprises at least 27 biomarkers selected from the group of biomarkers of Table 1A or of Table IB. In one embodiment, the molecular signature or profile of the silent phase of Alzheimer's disease, of grade SI, of grade S2 and/or of grade S3 comprises 27 biomarkers selected from the group of biomarkers of Table 1A or of Table IB. In one embodiment, the molecular signature or profile of the silent phase of Alzheimer's disease, of grade SI, of grade S2 and/or of grade S3 consists of 27 biomarkers selected from the group of biomarkers of Table 1A or of Table IB.

In one embodiment, the molecular signature or profile of the silent phase of Alzheimer's disease, of grade SI, of grade S2 and/or of grade S3 comprises at least 27 biomarkers selected from the group of biomarkers of Table 2A, Table 2B or Table 2C. In one embodiment, the molecular signature or profile of the silent phase of Alzheimer's disease, of grade SI, of grade S2 and/or of grade S3 comprises 27 biomarkers selected from the group of biomarkers of Table 2A, Table 2B or Table 2C. In one embodiment, the molecular signature or profile of the silent phase of Alzheimer's disease, of grade SI, of grade S2 and/or of grade S3 consists of 27 biomarkers selected from the group of biomarkers of Table 2A, Table 2B or Table 2C.

In one embodiment, the molecular signature or profile of the silent phase of Alzheimer's disease, of grade SI, of grade S2 and/or of grade S3 comprises at least 28 biomarkers selected from the group of biomarkers of Table 1A or of Table IB. In one embodiment, the molecular signature or profile of the silent phase of Alzheimer's disease, of grade SI, of grade S2 and/or of grade S3 comprises 28 biomarkers selected from the group of biomarkers of Table 1A or of Table IB. In one embodiment, the molecular signature or profile of the silent phase of Alzheimer's disease, of grade SI, of grade S2 and/or of grade S3 consists of 28 biomarkers selected from the group of biomarkers of Table 1A or of Table IB.

In one embodiment, the molecular signature or profile of the silent phase of Alzheimer's disease, of grade SI, of grade S2 and/or of grade S3 comprises at least 28 biomarkers selected from the group of biomarkers of Table 2A, Table 2B or Table 2C. In one embodiment, the molecular signature or profile of the silent phase of Alzheimer's disease, of grade SI, of grade S2 and/or of grade S3 comprises 28 biomarkers selected from the group of biomarkers of Table 2A, Table 2B or Table 2C. In one embodiment, the molecular signature or profile of the silent phase of Alzheimer's disease, of grade SI, of grade S2 and/or of grade S3 consists of 28 biomarkers selected from the group of biomarkers of Table 2A, Table 2B or Table 2C.

In one embodiment, the molecular signature or profile of the silent phase of Alzheimer's disease, of grade SI, of grade S2 and/or of grade S3 comprises at least 29 biomarkers selected from the group of biomarkers of Table 1A or of Table IB. In one embodiment, the molecular signature or profile of the silent phase of Alzheimer's disease, of grade SI, of grade S2 and/or of grade S3 comprises 29 biomarkers selected from the group of biomarkers of Table 1A or of Table IB. In one embodiment, the molecular signature or profile of the silent phase of Alzheimer's disease, of grade SI, of grade S2 and/or of grade S3 consists of 29 biomarkers selected from the group of biomarkers of Table 1A or of Table IB.

In one embodiment, the molecular signature or profile of the silent phase of Alzheimer's disease, of grade SI, of grade S2 and/or of grade S3 comprises at least 29 biomarkers selected from the group of biomarkers of Table 2A, Table 2B or Table 2C. In one embodiment, the molecular signature or profile of the silent phase of Alzheimer's disease, of grade SI, of grade S2 and/or of grade S3 comprises 29 biomarkers selected from the group of biomarkers of Table 2A, Table 2B or Table 2C. In one embodiment, the molecular signature or profile of the silent phase of Alzheimer's disease, of grade SI, of grade S2 and/or of grade S3 consists of 29 biomarkers selected from the group of biomarkers of Table 2A, Table 2B or Table 2C.

In one embodiment, the molecular signature or profile of the silent phase of Alzheimer's disease, of grade SI, of grade S2 and/or of grade S3 comprises at least 30 biomarkers selected from the group of biomarkers of Table 1A or of Table IB. In one embodiment, the molecular signature or profile of the silent phase of Alzheimer's disease, of grade SI, of grade S2 and/or of grade S3 comprises 30 biomarkers selected from the group of biomarkers of Table 1A or of Table IB. In one embodiment, the molecular signature or profile of the silent phase of Alzheimer's disease, of grade SI, of grade S2 and/or of grade S3 consists of 30 biomarkers selected from the group of biomarkers of Table 1A or of Table IB.

In one embodiment, the molecular signature or profile of the silent phase of Alzheimer's disease, of grade SI, of grade S2 and/or of grade S3 comprises at least 30 biomarkers selected from the group of biomarkers of Table 2A, Table 2B or Table 2C. In one embodiment, the molecular signature or profile of the silent phase of Alzheimer's disease, of grade SI, of grade S2 and/or of grade S3 comprises 30 biomarkers selected from the group of biomarkers of Table 2A, Table 2B or Table 2C.

In one embodiment, the molecular signature or profile of the silent phase of Alzheimer's disease, of grade SI, of grade S2 and/or of grade S3 consists of 30 biomarkers selected from the group of biomarkers of Table 2A, Table 2B or Table 2C.

In one embodiment, the decision as to whether the level, amount or concentration a given biomarker—or as to whether the mean profile of biomarkers' level, amount or concentration—is characteristic of the silent phase of Alzheimer's disease, of grade SI, of grade S2 and/or of grade S3 is taken in comparison to a reference signature or profile. This reference signature or profile may be either implemented in the software or an overall median or other arithmetic mean across measurements may be built.

In one embodiment, the reference signature or profile can be relative to a signature or profile derived from population studies, including, without limitation, such subjects having similar age range, subjects in the same or similar ethnic group, similar cancer history and the like.

In one embodiment, the reference signature or profile is derived from the measurement of the levels, amounts or concentrations of biomarkers of Table 1A or of Table IB, in a reference sample derived or obtained from one or more reference subjects.

In one embodiment, the reference signature or profile is derived from the measurement of the levels, amounts or concentrations of biomarkers of Table 2A, Table 2B or Table 2C, in a reference sample derived or obtained from one or more reference subjects. In one embodiment, the reference subject is an animal, preferably a mammal.

Examples of mammals include, but are not limited to, humans, non-human primates (such as, e.g, chimpanzees, and other apes and monkey species), farm animals (such as, e.g, cattle, horses, sheep, goats, and swine), domestic animals (such as, e.g, rabbits, dogs, and cats), laboratory animals (such as, e.g, rats, mice and guinea pigs), and the like. The term does not denote a particular age or gender, unless explicitly stated otherwise.

In one embodiment, the reference subject is a primate, including human and non-human primates. In one embodiment, the reference subject is a human.

In one embodiment, the reference subject is a substantially healthy subject.

As used herein, a “substantially healthy subject” has not been previously or will not be diagnosed or identified as having or suffering from Alzheimer's disease. Preferably, a “substantially healthy subject” has not been previously or will not be diagnosed or identified as having or suffering from a silent phase of Alzheimer's disease. Preferably, a “substantially healthy subject” has not been previously or will not be diagnosed or identified as having any of Alzheimer's related mild cognitive impairment (MCI), Alzheimer's dementia, physiopathological features of grade SI, physiopathological features of grade S2 and physiopathological features of grade S3, as defined hereinabove. In one embodiment, the reference subject is a subject who has not been diagnosed or identified as having or suffering from Alzheimer's disease, neither ante-mortem nor post-mortem. In one embodiment, the reference subject is a subject who has not been diagnosed or identified as having or suffering from a silent phase of Alzheimer's disease, neither ante-mortem nor post-mortem. Preferably, the reference subject is a subject who has not been diagnosed or identified as having any of Alzheimer's related mild cognitive impairment (MCI), Alzheimer's dementia, physiopathological features of grade SI, physiopathological features of grade S2 and physiopathological features of grade S3, as defined hereinabove, neither ante-mortem nor post-mortem.

In one embodiment, the reference signature or profile is derived from the measurement of the levels, amounts or concentrations of biomarkers of Table 1A or of Table IB, in reference samples derived or obtained from reference subjects in a reference population.

In one embodiment, the reference signature or profile is derived from the measurement of the levels, amounts or concentrations of biomarkers of Table 2A, Table 2B or Table 2C, in reference samples derived or obtained from reference subjects in a reference population.

In one embodiment, the reference population comprises substantially healthy subjects, preferably at least 25, more preferably at least 30, more preferably at least 35, more preferably at least 40, more preferably at least 45, more preferably at least 50, more preferably at least 75, more preferably at least 100, more preferably at least 150, more preferably at least 200 and even more preferably at least 500 substantially healthy subjects, as defined hereinabove.

In one embodiment, the reference population comprises subjects who have not been diagnosed or identified as having or suffering from Alzheimer's disease, neither ante-mortem nor post-mortem, preferably at least 25, more preferably at least 30, more preferably at least 35, more preferably at least 40, more preferably at least 45, more preferably at least 50, more preferably at least 75, more preferably at least 100, more preferably at least 150, more preferably at least 200 and even more preferably at least 500 subjects who have not been diagnosed or identified as having or suffering from Alzheimer's disease, neither ante-mortem nor post-mortem. In one embodiment, the reference population comprises subjects who have not been diagnosed or identified as having or suffering from a silent phase of Alzheimer's disease, neither ante-mortem nor post-mortem, preferably at least 50, more preferably at least 100, more preferably at least 200 and even more preferably at least 500 subjects who have not been diagnosed or identified as having or suffering from a silent phase of Alzheimer's disease, neither ante-mortem nor post-mortem. In one embodiment, the reference population comprises subjects who have not been diagnosed or identified as having any of mild cognitive impairment (MCI), dementia, physiopathological features of grade SI, physiopathological features of grade S2 and physiopathological features of grade S3, as defined hereinabove, neither ante-mortem nor post-mortem, preferably at least 25, more preferably at least 30, more preferably at least 35, more preferably at least 40, more preferably at least 45, more preferably at least 50, more preferably at least 75, more preferably at least 100, more preferably at least 150, more preferably at least 200 and even more preferably at least 500 subjects who have not been diagnosed or identified as having any of mild cognitive impairment (MCI), dementia, physiopathological features of grade SI, physiopathological features of grade S2 and physiopathological features of grade S3, as defined hereinabove, neither ante-mortem nor post-mortem.

In one embodiment, the reference subject is a grade SI subject.

As used herein, a “grade SI subject” has been previously diagnosed or identified as having or suffering from a grade SI silent phase of Alzheimer's disease. Preferably, a “grade SI subject” has not been previously or will not be diagnosed or identified as having or suffering from a grade S2 or grade S3 silent phase of Alzheimer's disease. Preferably, a “grade SI subject” has been previously diagnosed or identified as having physiopathological features of grade SI but neither of the physiopathological features of grade S2 and physiopathological features of grade S3 as defined hereinabove, nor mild cognitive impairment (MCI) and dementia.

In one embodiment, the grade SI subject is an animal, preferably a mammal.

Examples of mammals include, but are not limited to, humans, non-human primates (such as, e.g, chimpanzees, and other apes and monkey species), farm animals (such as, e.g, cattle, horses, sheep, goats, and swine), domestic animals (such as, e.g, rabbits, dogs, and cats), laboratory animals (such as, e.g, rats, mice and guinea pigs), and the like. The term does not denote a particular age or gender.

In one embodiment, the reference subject is a subject who has been previously diagnosed or identified as having or suffering from a grade SI silent phase of Alzheimer's disease, either ante-mortem or post-mortem.

In one embodiment, the reference signature or profile is derived from the measurement of the levels, amounts or concentrations of biomarkers of Table 1A or of Table IB, in reference samples derived or obtained from reference subjects in a reference population.

In one embodiment, the reference signature or profile is derived from the measurement of the levels, amounts or concentrations of biomarkers of Table 2A, Table 2B or Table 2C, in reference samples derived or obtained from reference subjects in a reference population.

In one embodiment, the reference population comprises grade SI subjects, preferably at least 25, more preferably at least 30, more preferably at least 35, more preferably at least 40, more preferably at least 45, more preferably at least 50, more preferably at least 75, more preferably at least 100, more preferably at least 150, more preferably at least 200 and even more preferably at least 500 grade SI subjects, as defined hereinabove.

In one embodiment, the reference population comprises subjects who have been previously diagnosed or identified as having or suffering from a grade SI silent phase of Alzheimer's disease, either ante-mortem or post-mortem, preferably at least 25, more preferably at least 30, more preferably at least 35, more preferably at least 40, more preferably at least 45, more preferably at least 50, more preferably at least 75, more preferably at least 100, more preferably at least 150, more preferably at least 200 and even more preferably at least 500 deceased subjects who have been previously diagnosed or identified as having or suffering from a grade SI silent phase of Alzheimer's disease, either ante-mortem or post-mortem.

In one embodiment, the reference subject is a grade S2 subject.

As used herein, a “grade S2 subject” has been previously diagnosed or identified as having or suffering from a grade S2 silent phase of Alzheimer's disease. Preferably, a “grade S2 subject” has not been previously or will not be diagnosed or identified as having or suffering from a grade S3 silent phase of Alzheimer's disease. Preferably, a “grade S2 subject” has been previously diagnosed or identified as having physiopathological features of grade S2 but neither of the physiopathological features of grade S3 as defined hereinabove, nor Alzheimer's related mild cognitive impairment (MCI) and Alzheimer's dementia.

In one embodiment, the grade S2 subject is an animal, preferably a mammal.

Examples of mammals include, but are not limited to, humans, non-human primates (such as, e.g., chimpanzees, and other apes and monkey species), farm animals (such as, e.g, cattle, horses, sheep, goats, and swine), domestic animals (such as, e.g, rabbits, dogs, and cats), laboratory animals (such as, e.g, rats, mice and guinea pigs), and the like. The term does not denote a particular age or gender.

In one embodiment, the reference subject is a subject who has been previously diagnosed or identified as having or suffering from a grade S2 silent phase of Alzheimer's disease, either ante-mortem or post-mortem.

In one embodiment, the reference signature or profile is derived from the measurement of the levels, amounts or concentrations of biomarkers of Table 1A or of Table IB, in reference samples derived or obtained from reference subjects in a reference population.

In one embodiment, the reference signature or profile is derived from the measurement of the levels, amounts or concentrations of biomarkers of Table 2A, Table 2B or Table 2C, in reference samples derived or obtained from reference subjects in a reference population.

In one embodiment, the reference population comprises grade S2 subjects, preferably at least 25, more preferably at least 30, more preferably at least 35, more preferably at least 40, more preferably at least 45, more preferably at least 50, more preferably at least 75, more preferably at least 100, more preferably at least 150, more preferably at least 200 and even more preferably at least 500 grade S2 subjects, as defined hereinabove.

In one embodiment, the reference population comprises subjects who have been previously diagnosed or identified as having or suffering from a grade S2 silent phase of Alzheimer's disease, either ante-mortem or post-mortem, preferably at least 25, more preferably at least 30, more preferably at least 35, more preferably at least 40, more preferably at least 45, more preferably at least 50, more preferably at least 75, more preferably at least 100, more preferably at least 150, more preferably at least 200 and even more preferably at least 500 deceased subjects who have been previously diagnosed or identified as having or suffering from a grade S2 silent phase of Alzheimer's disease, either ante-mortem or post-mortem.

In one embodiment, the reference subject is a grade S3 subject.

As used herein, a “grade S3 subject” has been previously diagnosed or identified as having or suffering from a grade S3 silent phase of Alzheimer's disease. Preferably, a “grade S3 subject” has been previously diagnosed or identified as having physiopathological features of grade S3 but not Alzheimer's dementia. In some cases, Alzheimer's related MCI could be considered as grade S3 subject.

In one embodiment, the grade S3 subject is an animal, preferably a mammal.

Examples of mammals include, but are not limited to, humans, non-human primates (such as, e.g, chimpanzees, and other apes and monkey species), farm animals (such as, e.g, cattle, horses, sheep, goats, and swine), domestic animals (such as, e.g, rabbits, dogs, and cats), laboratory animals (such as, e.g, rats, mice and guinea pigs), and the like. The term does not denote a particular age or gender.

In one embodiment, the reference subject is a subject who has been previously diagnosed or identified as having or suffering from a grade S3 silent phase of Alzheimer's disease, either ante-mortem or post-mortem.

In one embodiment, the reference signature or profile is derived from the measurement of the levels, amounts or concentrations of biomarkers of Table 1A or of Table IB, in reference samples derived or obtained from reference subjects in a reference population.

In one embodiment, the reference signature or profile is derived from the measurement of the levels, amounts or concentrations of biomarkers of Table 2A, Table 2B or Table 2C, in reference samples derived or obtained from reference subjects in a reference population.

In one embodiment, the reference population comprises grade S3 subjects, preferably at least 25, more preferably at least 30, more preferably at least 35, more preferably at least 40, more preferably at least 45, more preferably at least 50, more preferably at least 75, more preferably at least 100, more preferably at least 150, more preferably at least 200 and even more preferably at least 500 grade S3 subjects, as defined hereinabove.

In one embodiment, the reference population comprises subjects who have been previously diagnosed or identified as having or suffering from a grade S3 silent phase of Alzheimer's disease, either ante-mortem or post-mortem, preferably at least 25, more preferably at least 30, more preferably at least 35, more preferably at least 40, more preferably at least 45, more preferably at least 50, more preferably at least 75, more preferably at least 100, more preferably at least 150, more preferably at least 200 and even more preferably at least 500 deceased subjects who have been previously diagnosed or identified as having or suffering from a grade S3 silent phase of Alzheimer's disease, either ante-mortem or post-mortem.

By implying a multitude of samples from the reference population, it is conceivable to calculate a median and/or mean level, amount or concentration for each biomarker of Table 1A or of Table IB (or of Table 2A, Table 2B or Table 2C)—or alternatively, to build a reference signature or profile of biomarkers of Table 1A or of Table IB's level, amount or concentration (or of Table 2 A, Table 2B or Table 2C's level, amount or concentration). In relation to these results, a respective level, amount or concentration of a given biomarker—or alternatively, a respective reference signature or profile of biomarkers' level, amount or concentration—can be monitored as being substantially different (such as substantially higher or substantially lower) or substantially similar In one embodiment, the reference signature or profile is constructed using algorithms and other methods of statistical and structural classification. Samples from the reference population are used to compute a mean profile on the at least 1 biomarker, preferably on the at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or more biomarkers selected from the group of biomarkers of Table 1A or of Table IB. In one embodiment, the reference signature or profile is constructed using algorithms and other methods of statistical and structural classification. Samples from the reference population are used to compute a mean profile on the at least 1 biomarker, preferably on the at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or more biomarkers selected from the group of biomarkers of Table 2A, Table 2B or Table 2C. These reference signatures or profiles are computed for four reference groups of (1) healthy subjects, (2) grade SI subjects, (3) grade S2 subjects, and (4) grade S4 subjects, and thereafter referred to as “group centroids”.

In one embodiment, the centroids are centered. In one embodiment, the centroids are scaled by biomarker. In one embodiment, the centroids are centered and scaled by biomarker.

Cancer class prediction from gene expression profiling based on a centroid classification is a technic well-known from the one skilled in the art. Reference can be made, e.g, to Tibshirani et al., 2002. Proc Natl Acad Sci U S A. 99(10):6567-72; Dabney, 2005. Bioinformatics. 21(22):4148-54; and Shen et al., 2009. J Biomed Inform. 42(1):59-65.

In one embodiment, the molecular signature or profile of the invention is characteristic of the silent phase of Alzheimer's disease, of grade SI, of grade S2 and/or of grade S3 if the level, amount or concentration of at least 1 biomarker, preferably at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or more biomarkers selected from the group of biomarkers of Table 1A or of Table IB (or of Table 2A, Table 2B or Table 2C), varies as described in Table 3, when taking in comparison to a reference signature or profile derived or obtained from substantially healthy subjects.

TABLE 3
BIOMARKER VARIATION PROFILE OF THE SILENT PHASE
OF ALZHEIMER'S DISEASE IN GRADE S1, GRADE
S2, AND GRADE S3, VERSUS SUBSTANTIALLY HEALTHY

| | Grade S1 | Grade S2 | Grade S3 |
|---|---|---|---|
| Biomarkers | vs healthy | vs healthy | vs healthy |
| 14-3-3 protein beta/alpha | − | = | = |
| 14-3-3 protein epsilon | − | − | − |
| 14-3-3 protein eta | − | + | − |
| 14-3-3 protein gamma | − | − | − |
| 14-3-3 protein theta | − | − | − |
| 14-3-3 protein zeta/delta | − | + | − |
| Apolipoprotein A-I | − | = | = |
| Apolipoprotein A-II | + | + | = |
| Apolipoprotein A-IV | + | − | − |
| Apolipoprotein B-100 | = | = | − |
| Apolipoprotein C-I | + | + | − |
| Apolipoprotein C-II (predicted) | + | + | − |
| Apolipoprotein C-III | + | + | + |
| Apolipoprotein C-IV | + | + | + |
| Apolipoprotein D | − | − | + |
| Apolipoprotein E | − | + | + |
| Rat apolipoprotein E protein | − | − | + |
| Apolipoprotein H (beta-2- | + | + | − |
| glycoprotein I) | | | |
| Apolipoprotein M | − | + | − |
| Apolipoprotein N | + | + | − |
| Actin-related protein 2 | + | − | + |
| Actin-related protein 2/3 complex | + | − | − |
| subunit 1B | | | |
| Actin-related protein 2/3 complex | − | + | − |
| subunit 3 | | | |
| Actin-related protein 2/3 complex | = | + | + |
| subunit 4 | | | |
| Actin-related protein 2/3 complex | + | − | = |
| subunit 5 | | | |
| Actin-related protein 3 | + | = | − |
| Arp2/3 complex 34 kDa subunit | + | + | + |
| Carboxylesterase 1C | + | − | = |
| Carboxylesterase 1E | + | − | − |
| 2-methylbutyrylcarnitine (C5) | = | − | = |
| Acetylcarnitine (C2) | − | = | − |
| Arachidonoylcarnitine (C20:4) | − | + | − |
| Butyrylcarnitine (C4) | = | − | − |
| Carnitine | − | + | − |
| Cis-4-decenoylcarnitine (C10:1) | − | + | − |
| Isobutyrylcarnitine (C4) | − | − | − |
| Isovalerylcarnitine (C5) | − | − | − |
| Laurylcarnitine (C12) | − | − | + |
| Linoleoylcarnitine (C18:2) | − | + | = |
| Myristoylcarnitine (C14) | − | = | + |
| Octanoylcarnitine (C8) | − | − | = |
| Oleoylcarnitine (C18) | − | + | = |
| Palmitoleoylcarnitine (C16:1) | − | = | + |
| Palmitoylcarnitine (C16) | − | + | − |
| Propionylcarnitine (C3) | + | − | − |
| Stearoylcarnitine (C18) | − | + | − |
| Stearoylcarnitine (C18) | − | + | − |
| Beta-muricholate | − | + | − |
| Chenodeoxycholate | = | + | − |
| Cholate | − | + | − |
| Deoxycholate | − | = | − |
| Glycocholate | − | + | − |
| Taurochenodeoxycholate | − | + | + |
| Taurocholate | − | + | + |
| Taurodeoxycholate | − | + | − |
| Coagulation factor IX | + | − | = |
| Coagulation factor VII | − | − | + |
| Coagulation factor X | + | + | − |
| Coagulation factor XI | + | + | − |
| Coagulation factor XII | − | = | + |
| Coagulation factor XIII A chain | − | + | − |
| Coagulation factor XIII B chain | + | + | − |
| Complement C1q subcomponent | − | − | + |
| subunit A | | | |
| Complement C1q subcomponent | − | − | + |
| subunit B | | | |
| Complement C1q subcomponent | − | − | + |
| subunit C | | | |
| Complement C1s subcomponent | = | − | = |
| Complement C1s subcomponent | = | + | + |
| Complement C2 | − | − | − |
| Complement C3 | + | − | + |
| Complement C4 | − | + | + |
| Complement C4B | − | − | + |
| Complement C4-like | − | = | + |
| Complement C5 | − | + | + |
| Complement C7 | + | − | − |
| Complement C8 alpha chain | = | + | − |
| Complement C8 gamma chain | − | + | − |
| Complement factor H-related protein | = | = | + |
| Creatine kinase B-type | + | = | − |
| Creatine kinase M-type | − | + | − |
| Globin a2 | − | − | + |
| Globin a4 | − | − | + |
| Globin c2 | − | − | + |
| Globin c3 | − | − | + |
| Globin d1 | − | − | = |
| Myoglobin | + | − | = |
| Alpha-2 antiplasmin | + | + | − |
| Murinoglobulin-2 | + | + | − |
| Ig kappa chain C | − | + | − |
| Igh-6 protein | + | = | − |
| Alpha-2-macroglobulin | − | − | − |
| Complement factor properdin | − | − | − |
| Haptoglobin | + | + | − |
| Beta-2-microglobulin | − | + | − |
| Ceruloplasmin | − | − | − |
| Serotransferrin | + | − | = |
| Similar to immunoglobulin kappa- | = | + | = |
| chain VK-1 | | | |
| Serine (or cysteine) proteinase | − | − | + |
| inhibitor clade A member 4 | | | |
| Alpha-2-macroglobulin | − | + | + |
| IgG-2a protein | + | − | + |
| Prothrombin | − | + | + |
| Alpha-1-macroglobulin | + | + | + |
| Serum albumin | + | − | + |
| Immunoglobulin heavy chain variable | − | − | + |
| region | | | |
| Corticosteroid-binding globulin | + | = | + |
| Ig heavy chain V region IR2 | − | − | + |
| Murinoglobulin-2 | + | + | + |
| Ig gamma-2B chain C region | − | = | + |
| Igh-6 protein | + | − | + |
| Ig lambda-2 chain C region | − | − | + |
| Ig delta chain C region | − | − | + |
| Ig gamma-2C chain C region | − | − | + |
| Igh-6 protein | − | + | + |
| Ig lambda-2 chain C region | − | − | + |
| Ig delta chain C region | − | − | + |
| Ig gamma-2C chain C region | − | − | + |
| Igh-6 protein | − | + | + |
| Immunoglobulin J chain | − | + | + |
| Ig kappa chain V region S211 | = | − | + |
| Seram amyloid A protein | − | − | − |
| Kininogen | − | + | + |
| Kininogen 1 | − | + | + |
| Carboxylesterase 1 | − | + | + |
| T-kininogen 2 | − | + | + |
| 5-hydroxylysine | + | − | − |
| Fructosyllysine | + | + | + |
| Gamma-glutamyl-alpha-lysine | + | − | − |
| Lysine | + | − | − |
| N⁶,N⁶,N⁶-trimethyllysine | − | = | + |
| N⁶-acetyllysine | + | − | − |
| N⁶-methyllysine | + | − | − |
| N,N,N-trimethyl-5-aminovalerate | + | − | − |
| Pipecolate | = | + | − |
| Proteasome subunit alpha type | − | = | − |
| Proteasome subunit alpha type-7 | − | = | − |
| Proteasome subunit alpha type-1 | − | − | − |
| Proteasome subunit alpha type-2 | − | − | − |
| Proteasome subunit alpha type-3 | = | + | − |
| Proteasome subunit alpha type-4 | − | − | − |
| Proteasome subunit alpha type-6 | + | + | + |
| Proteasome subunit beta | − | − | + |
| Proteasome subunit beta type | − | − | + |
| Proteasome subunit beta type-1 | − | = | = |
| Proteasome subunit beta type-10 | − | + | − |
| Proteasome subunit beta type-3 | + | − | + |
| Alpha-1-antiproteinase | + | − | − |
| Heparin cofactor 2 | + | − | + |
| Plasma protease C1 inhibitor | − | − | + |
| Protein Z-dependent protease | − | + | − |
| inhibitor | | | |
| Serine (or cysteine) peptidase | − | + | = |
| inhibitor clade B member 10 | | | |
| Serine (or cysteine) peptidase | − | + | + |
| inhibitor clade B member 6a | | | |
| Serine (or cysteine) peptidase | + | + | − |
| inhibitor clade C member 1 | | | |
| Serine protease inhibitor A3K | = | = | = |
| Serine protease inhibitor A3L | = | = | = |
| Serine protease inhibitor A3M | + | − | − |
| Serine protease inhibitor A3N | − | + | − |
| Serine protease inhibitor Kazal-type | − | − | + |
| 3-like | | | |
| Serpin A11 | − | − | + |
| Serpin family F member 2 | + | + | − |
| Thyroxine-binding globulin | + | + | = |
| 2,3-dihydroxyisovalerate | − | − | − |
| 2-hydroxy-3-methylvalerate | − | − | + |
| 3-methyl-2-oxovalerate | + | − | + |
| Alpha-hydroxyisovalerate | − | − | + |
| Beta-hydroxyisovalerate | + | − | − |
| 10 kDa heat shock protein, | − | + | − |
| mitochondrial | | | |
| 1-methyl-5-imidazoleacetate | − | + | + |
| 3-methyl-2-oxobutyrate | + | − | + |
| 5-methyl-2′-deoxycytidine | = | + | + |
| 5-methylthioadenosine (MTA) | − | + | − |
| 5-oxoproline | − | − | − |
| Adenylate kinase 4, mitochondrial | − | + | − |
| Allantoic acid | + | + | + |
| Alpha-1B-glycoprotein | = | − | + |
| Alpha-actinin-1 | = | + | = |
| Alpha-soluble NSF attachment | − | + | + |
| protein | | | |
| Anserine | = | − | − |
| Anti-F4/80 kappa light chain variable | − | − | + |
| region | | | |
| Biotinidase | + | + | − |
| BWK3 | − | − | + |
| C4b-binding protein beta chain | − | + | − |
| Calpain small subunit 1 | − | + | − |
| Calreticulin | − | − | − |
| Carboxypeptidase B2 | − | − | − |
| Carnosine | + | − | − |
| Chromogranin A | − | − | − |
| Clathrin heavy chain | − | − | − |
| Coactosin-like protein | − | = | − |
| Complement C6 | − | − | = |
| Dimethylarginine | + | − | − |
| EGF-containing fibulin-like | + | + | = |
| extracellular matrix protein 1 | | | |
| EGF-containing fibulin-like | + | + | = |
| extracellular matrix protein 1 | | | |
| EH domain-containing protein 3 | − | + | + |
| Elongation factor 1-alpha | + | + | + |
| Ergothioneine | − | = | + |
| Ethylmalonate | + | + | − |
| Extracellular matrix protein 1 | + | + | − |
| F-actin-capping protein subunit | − | − | + |
| alpha-2 | | | |
| Fibronectin 1 | + | + | − |
| Fibulin-1 | + | + | − |
| Fibulin-5 | + | = | − |
| Fructose | − | − | + |
| Fructose-bisphosphate aldolase | + | + | − |
| FYN-binding protein 1 | − | − | − |
| Gelsolin | + | + | − |
| Glucuronate | − | + | + |
| Glycosylphosphatidylinositol specific | + | + | − |
| phospholipase D1 | | | |
| HGF activator | − | − | + |
| Histidine-rich glycoprotein | − | − | + |
| Hyaluronidase | − | − | − |
| Hydroxyproline | + | − | − |
| Ighg protein | = | − | = |
| Integrin beta | − | + | − |
| Integrin subunit alpha V | − | + | + |
| Interleukin 1 receptor accessory | − | − | + |
| protein | | | |
| Keratin, type II cytoskeletal 5 | + | + | + |
| Lipase | + | − | − |
| Lumican | + | − | − |
| Lysyl oxidase-like 1 | = | + | − |
| Macrophage migration inhibitory | − | + | − |
| factor | | | |
| Major urinary protein | + | = | − |
| Malate dehydrogenase, cytoplasmic | − | − | − |
| Mannan-binding lectin serine | + | = | − |
| peptidase 2 | | | |
| Mannose-binding protein A | + | − | + |
| Microfibril-associated glycoprotein 4 | − | + | − |
| Multiple inositol polyphosphate | − | − | − |
| phosphatase 1 | | | |
| Myosin regulatory light polypeptide 9 | − | + | − |
| Myosin regulatory light chain RLC-A | + | + | + |
| N-acetylasparagine | − | − | − |
| Octadecanedioate (C18) | − | − | + |
| Oleate/vaccenate (18:1) | − | = | + |
| Palmitate (16:0) | − | + | + |
| Palmitoleate (16:1n7) | − | − | + |
| Peptidyl-prolyl cis-trans isomerase A | + | + | − |
| Peptidyl-prolyl cis-trans isomerase | + | + | + |
| FKBP1A | | | |
| Peroxiredoxin 3 | − | + | − |
| Phenylalanine | − | − | + |
| Polyubiquitin-C | − | = | + |
| Prolylhydroxyproline | + | − | − |
| Rat MHC class I truncated cell | − | − | + |
| surface antigen mRNA | | | |
| Retinoic acid receptor responder 2 | + | + | − |
| Retinol-binding protein | + | − | − |
| Ribonate (ribonolactone) | − | − | − |
| Ribulonate/xylulonate/lyxonate | − | − | = |
| Sacsin molecular chaperone | + | + | − |
| Serum amyloid P-component | − | + | + |
| Sulfate | − | − | − |
| Superoxide dismutase [Mn], | − | + | − |
| mitochondrial | | | |
| Talin 2 | − | − | − |
| Thioredoxin | − | − | + |
| Tmprss13 protein | + | + | + |
| Transferrin receptor protein 1 | − | − | + |
| Transthyretin | + | − | − |
| Urinary protein 1 | + | − | − |
| Voltage-dependent anion-selective | − | − | + |
| channel protein 3 | | | |
| Xaa-Pro aminopeptidase 2 | + | − | + |

“+” means substantially higher (i.e., >+5% compared to substantially healthy);
“−” means substantially lower (i.e., >−5% compared to substantially healthy);
“=” means substantially similar (i.e., <±5% compared to substantially healthy).

In one embodiment, the molecular signature or profile of the invention is characteristic of grade SI of the silent phase of Alzheimer's disease if the level, amount or concentration of at least 1 biomarker, preferably at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or more biomarkers selected from the group of biomarkers of Table 4A is substantially lower (i.e., is more than 5% lower), when taking in comparison to the level, amount or concentration of the same biomarker(s) in a substantially healthy subject or a population of substantially healthy subjects.

TABLE 4A

| | |
|---|---|
| 14-3-3 protein beta/alpha | Carboxylesterase 1 |
| 14-3-3 protein epsilon | T-kininogen 2 |
| 14-3-3 protein eta | N⁶,N⁶,N⁶-trimethyllysine |
| 14-3-3 protein gamma | Proteasome subunit alpha type |
| 14-3-3 protein theta | Proteasome subunit alpha type-7 |
| 14-3-3 protein zeta/delta | Proteasome subunit alpha type-1 |
| Apolipoprotein A-I | Proteasome subunit alpha type-2 |
| Apolipoprotein D | Proteasome subunit alpha type-4 |
| Apolipoprotein E | Proteasome subunit beta |
| Rat apolipoprotein E protein | Proteasome subunit beta type |
| Apolipoprotein M | Proteasome subunit beta type-1 |
| Actin-related protein 2/3 complex subunit | Proteasome subunit beta type-10 |
| 3 | |
| Acetylcarnitine (C2) | Protein Z-dependent protease inhibitor |
| Arachidonoylcarnitine (C20:4) | Serine (or cysteine) peptidase inhibitor |
| | clade B member 10 |
| Carnitine | Serine (or cysteine) peptidase inhibitor |
| | clade B member 6a |
| Cis-4-decenoylcarnitine (C10:1) | Serine protease inhibitor A3N |
| Isobutyrylcarnitine (C4) | Serpin A11 |
| Isovalerylcarnitine (C5) | 2,3-dihydroxyisovalerate |
| Laurylcarnitine (C12) | 2-hydroxy-3-methylvalerate |
| Linoleoylcarnitine (C18:2) | Alpha-hydroxyisovalerate |
| Myristoylcarnitine (C14) | 10 kDa heat shock protein, mitochondrial |
| Octanoylcarnitine (C8) | 1-methyl-5-imidazoleacetate |
| Oleoylcarnitine (C18) | 5-methylthioadenosine (MTA) |
| Palmitoleoylcarnitine (C16:1) | 5-oxoproline |
| Palmitoylcarnitine (C16) | Adenylate kinase 4, mitochondrial |
| Stearoylcarnitine (C18) | Alpha-soluble NSF attachment protein |
| Beta-muricholate | Anti-F4/80 kappa light chain variable |
| | region |
| Cholate | BWK3 |
| Deoxycholate | C4b-binding protein beta chain |
| Glycocholate | Calpain small subunit 1 |
| Taurochenodeoxycholate | Calreticulin |
| Taurocholate | Carboxypeptidase B2 |
| Taurodeoxycholate | Chromogranin A |
| Coagulation factor VII | Clathrin heavy chain |
| Coagulation factor XII | Coactosin-like protein |
| Coagulation factor XIII A chain | Complement C6 |
| Complement C1q subcomponent subunit | EH domain-containing protein 3 |
| A | |
| Complement C1q subcomponent subunit | Ergothioneine |
| B | |
| Complement C1q subcomponent subunit | F-actin-capping protein subunit alpha-2 |
| C | |
| Complement C2 | Fructose |
| Complement C4 | FYN-binding protein 1 |
| Complement C4B | Glucuronate |
| Complement C4-like | HGF activator |
| Complement C5 | Histidine-rich glycoprotein |
| Complement C8 gamma chain | Hyaluronidase |
| Creatine kinase M-type | Integrin beta |
| Globin a2 | Integrin subunit alpha V |
| Globin a4 | Interleukin 1 receptor accessory protein |
| Globin c2 | Macrophage migration inhibitory factor |
| Globin c3 | Malate dehydrogenase, cytoplasmic |
| Globin d1 | Microfibril-associated glycoprotein 4 |
| Vitamin K-dependent protein C | Multiple inositol polyphosphate |
| | phosphatase 1 |
| Ig kappa chain C | Myosin regulatory light polypeptide 9 |
| Alpha-2-macroglobulin | N-acetylasparagine |
| Complement factor properdin | Octadecanedioate (C18) |
| Beta-2-microglobulin | Oleate/vaccenate (18:1) |
| Ceruloplasmin | Palmitate (16:0) |
| Same (or cysteine) proteinase inhibitor | Palmitoleate (16:1n7) |
| clade A member 4 | |
| Alpha-2-macroglobulin | Peroxiredoxin 3 |
| Prothrombin | Phenylalanine |
| Immunoglobulin heavy chain variable | Polyubiquitin-C |
| region | |
| Ig heavy chain V region IR2 | Rat MHC class I truncated cell surface |
| | antigen mRNA |
| Ig gamma-2B chain C region | Ribonate (ribonolactone) |
| Ig lambda-2 chain C region | Ribulonate/xylulonate/lyxonate |
| Ig delta chain C region | Serum amyloid P-component |
| Ig gamma-2C chain C region | Sulfate |
| Igh-6 protein | Superoxide dismutase [Mn], |
| | mitochondrial |
| Immunoglobulin J chain | Talin 2 |
| Serum amyloid A protein | Thioredoxin |
| Kininogen | Transferrin receptor protein 1 |
| Kininogen 1 | Voltage-dependent anion-selective |
| | channel protein 3 |

In one embodiment, the molecular signature or profile of the invention is characteristic of grade SI of the silent phase of Alzheimer's disease if the level, amount or concentration of at least 1 biomarker, preferably at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or more biomarkers selected from the group of biomarkers of Table 4B is substantially higher (i.e., is more than 5% higher), when taking in comparison to the level, amount or concentration of the same biomarker(s) in a substantially healthy subject or a population of substantially healthy subjects.

TABLE 4B

| | |
|---|---|
| Apolipoprotein A-II | N,N,N-trimethyl-5-aminovalerate |
| Apolipoprotein A-IV | Proteasome subunit alpha type-6 |
| Apolipoprotein C-I | Proteasome subunit beta type-3 |
| Apolipoprotein C-II (predicted) | Alpha-1-antiproteinase |
| Apolipoprotein C-III | Heparin cofactor 2 |
| Apolipoprotein C-IV | Serine (or cysteine) peptidase inhibitor |
| | clade C member 1 |
| Apolipoprotein H (beta-2-glycoprotein I) | Serine protease inhibitor A3M |
| Apolipoprotein N | Serpin family F member 2 |
| Actin-related protein 2 | Thyroxine-binding globulin |
| Actin-related protein 2/3 complex subunit | 3-methyl-2-oxovalerate |
| 1B | |
| Actin-related protein 2/3 complex subunit | Beta-hydroxyisovalerate |
| 5 | |
| Actin-related protein 3 | 3-methyl-2-oxobutyrate |
| Arp2/3 complex 34 kDa subunit | Allantoic acid |
| Carboxylesterase 1C | Biotinidase |
| Carboxylesterase 1E | Carnosine |
| Propionylcarnitine (C3) | Dimethylarginine |
| Coagulation factor IX | EGF-containing fibulin-like extracellular |
| | matrix protein 1 |
| Coagulation factor X | Elongation factor 1-alpha |
| Coagulation factor XI | Ethylmalonate |
| Coagulation factor XIII B chain | Extracellular matrix protein 1 |
| Complement C3 | Fibronectin 1 |
| Complement C7 | Fibulin-1 |
| Creatine kinase B-type | Fibulin-5 |
| Myoglobin | Fructose-bisphosphate aldolase |
| Alpha-2 antiplasmin | Gelsolin |
| Murinoglobulin-2 | Glycosylphosphatidylinositol specific |
| | phospholipase D1 |
| Igh-6 protein | Lumican |
| Haptoglobin | Major urinary protein |
| Serotransferrin | Mannan-binding lectin serine peptidase 2 |
| IgG-2a protein | Mannose-binding protein A |
| Alpha-1-macroglobulin | Myosin regulatory light chain RLC-A |
| Serum albumin | Peptidyl-prolyl cis-trans isomerase A |
| Corticosteroid-binding globulin | Peptidyl-prolyl cis-trans isomerase |
| | FKBP1A |
| Murinoglobulin-2 | Prolylhydroxyproline |
| Igh-6 protein | Retinoic acid receptor responder 2 |
| 5-hydroxylysine | Retinol-binding protein |
| Fructosyllysine | Sacsin molecular chaperone |
| Gamma-glutamyl-alpha-lysine | Tmprss13 protein |
| Lysine | Transthyretin |
| N⁶-acetyllysine | Urinary protein 1 |
| N⁶-methyllysine | Xaa-Pro aminopeptidase 2 |

In one embodiment, the molecular signature or profile of the invention is characteristic of grade SI of the silent phase of Alzheimer's disease if the level, amount or concentration of at least 1 biomarker, preferably at least 2, 3, 4, 5, 6, 7, 8, 9, 10, 11, 12, 13, 14, 15, 16, 17, 18, 19, 20, 21, 22, or 23 biomarkers selected from the group of biomarkers of Table 4C is substantially similar (i.e., is no more than 5% lower or higher), when taking in comparison to the level, amount or concentration of the same biomarker(s) in a substantially healthy subject or a population of substantially healthy subjects.

TABLE 4C

| | |
|---|---|
| Apolipoprotein B-100 | Proteasome subunit alpha type-3 |
| Actin-related protein 2/3 complex subunit | Plasma protease C1 inhibitor |
| 4 | |
| 2-methylbutyrylcarnitine (C5) | Serine protease inhibitor A3K |
| Butyrylcarnitine (C4) | Serine protease inhibitor A3L |
| Chenodeoxycholate | Serine protease inhibitor Kazal-type 3- |
| | like |
| Complement C1s subcomponent | 5-methyl-2′-deoxycytidine |
| Complement C1s subcomponent | Alpha-1B-glycoprotein |
| Complement factor H-related protein | Alpha-actinin-1 |
| Complement C8 alpha chain | Anserine |
| Similar to immunoglobulin kappa-chain | Ighg protein |
| VK-1 | |
| Ig kappa chain V region S211 | Lysyl oxidase-like 1 |
| Pipecolate | |

In one embodiment, the molecular signature or profile of the invention is characteristic of grade SI of the silent phase of Alzheimer's disease if: the level, amount or concentration of at least 1 biomarker, preferably at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or more biomarkers selected from the group of biomarkers of Table 4A is substantially lower (i.e., is more than 5% lower), when taking in comparison to the level, amount or concentration of the same biomarker(s) in a substantially healthy subject or a population of substantially healthy subjects, the level, amount or concentration of at least 1 biomarker, preferably at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or more biomarkers selected from the group of biomarkers of Table 4B is substantially higher (i.e., is more than 5% higher), when taking in comparison to the level, amount or concentration of the same biomarker(s) in a substantially healthy subject or a population of substantially healthy subjects, and/or the level, amount or concentration of at least 1 biomarker, preferably at least 2, 3, 4, 5, 6, 7, 8, 9, 10, 11, 12, 13, 14, 15, 16, 17, 18, 19, 20, 21, 22, or 23 biomarkers selected from the group of biomarkers of Table 4C is substantially similar (i.e., is no more than 5% lower or higher), when taking in comparison to the level, amount or concentration of the same biomarker(s) in a substantially healthy subject or a population of substantially healthy subjects.

In one embodiment, the molecular signature or profile of the invention is characteristic of grade S2 of the silent phase of Alzheimer's disease if the level, amount or concentration of at least 1 biomarker, preferably at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or more biomarkers selected from the group of biomarkers of Table 5A is substantially lower (i.e., is more than 5% lower), when taking in comparison to the level, amount or concentration of the same biomarker(s) in a substantially healthy subject or a population of substantially healthy subjects.

TABLE 5A

| | |
|---|---|
| 14-3-3 protein epsilon | Phenylalanine |
| 14-3-3 protein gamma | Rat MHC class I truncated cell surface |
| | antigen mRNA |
| 14-3-3 protein theta | Ribonate (ribonolactone) |
| Apolipoprotein D | Ribulonate/xylulonate/lyxonate |
| Rat apolipoprotein E protein | Sulfate |
| Isobutyrylcarnitine (C4) | Talin 2 |
| Isovalerylcarnitine (C5) | Thioredoxin |
| Laurylcarnitine (C12) | Transferrin receptor protein 1 |
| Octanoylcarnitine (C8) | Voltage-dependent anion-selective |
| | channel protein 3 |
| Coagulation factor VII | Apolipoprotein A-IV |
| Complement C1q subcomponent subunit A | Actin-related protein 2 |
| Complement C1q subcomponent subunit B | Actin-related protein 2/3 complex |
| | subunit 1B |
| Complement C1q subcomponent subunit C | Actin-related protein 2/3 complex |
| | subunit 5 |
| Complement C2 | Carboxylesterase 1C |
| Complement C4B | Carboxylesterase 1E |
| Globin a2 | Propionylcarnitine (C3) |
| Globin a4 | Coagulation factor IX |
| Globin c2 | Complement C3 |
| Globin c3 | Complement C7 |
| Globin d1 | Myoglobin |
| Alpha-2-macroglobulin | Angiotensinogen |
| Complement factor properdin | Serotransferrin |
| Ceruloplasmin | IgG-2a protein |
| Serine (or cysteine) proteinase inhibitor | Serum albumin |
| clade A member 4 | |
| Immunoglobulin heavy chain variable | Igh-6 protein |
| region | |
| Ig heavy chain V region IR2 | 5-hydroxylysine |
| Ig lambda-2 chain C region | Gamma-glutamyl-alpha-lysine |
| Ig delta chain C region | Lysine |
| Ig gamma-2C chain C region | N⁶-acetyllysine |
| Seram amyloid A protein | N⁶-methyllysine |
| Proteasome subunit alpha type-1 | N,N,N-trimethyl-5-aminovalerate |
| Proteasome subunit alpha type-2 | Proteasome subunit beta type-3 |
| Proteasome subunit alpha type-4 | Alpha-1-antiproteinase |
| Proteasome subunit beta | Heparin cofactor 2 |
| Proteasome subunit beta type | Serine protease inhibitor A3M |
| Serpin A11 | 3-methyl-2-oxovalerate |
| 2,3-dihydroxyisovalerate | Beta-hydroxyisovalerate |
| 2-hydroxy-3-methylvalerate | 3-methyl-2-oxobutyrate |
| Alpha-hydroxyisovalerate | Carnosine |
| 5-oxoproline | Dimethylarginine |
| Anti-F4/80 kappa light chain variable region | Hydroxyproline |
| BWK3 | Lipase |
| Calreticulin | Lumican |
| Carboxypeptidase B2 | Mannose-binding protein A |
| Chromogranin A | Prolylhydroxyproline |
| Clathrin heavy chain | Retinol-binding protein |
| Complement C6 | Transthyretin |
| F-actin-capping protein subunit alpha-2 | Urinary protein 1 |
| Fructose | Xaa-Pro aminopeptidase 2 |
| Fructose | Xaa-Pro aminopeptidase 2 |
| FYN-binding protein 1 | 2-methylbutyrylcarnitine (C5) |
| HGF activator | Butyrylcarnitine (C4) |
| Histidine-rich glycoprotein | Complement C1s subcomponent |
| Hyaluronidase | Ig kappa chain V region S211 |
| Interleukin I receptor accessory protein | Plasma protease C1 inhibitor |
| Malate dehydrogenase, cytoplasmic | Serine protease inhibitor Kazal-type 3- |
| | like |
| Multiple inositol polyphosphate phosphatase | Alpha-1B-glycoprotein |
| I | |
| N-acetylasparagine | Anserine |
| Octadecanedioate (C18) | Ighg protein |
| Palmitoleate (16:1n7) | |

In one embodiment, the molecular signature or profile of the invention is characteristic of grade S2 of the silent phase of Alzheimer's disease if the level, amount or concentration of at least 1 biomarker, preferably at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or more biomarkers selected from the group of biomarkers of Table 5B is substantially higher (i.e., is more than 5% higher), when taking in comparison to the level, amount or concentration of the same biomarker(s) in a substantially healthy subject or a population of substantially healthy subjects.

TABLE 5B

| | |
|---|---|
| 14-3-3 protein eta | Serum amyloid P-component |
| 14-3-3 protein zeta/delta | Superoxide dismutase [Mn], |
| | mitochondrial |
| Apolipoprotein E | Apolipoprotein A-II |
| Apolipoprotein M | Apolipoprotein C-I |
| Actin-related protein 2/3 complex subunit | Apolipoprotein C-II (predicted) |
| 3 | |
| Arachidonoylcarnitine (C20:4) | Apolipoprotein C-IIII |
| Carnitine | Apolipoprotein C-IV |
| Cis-4-decenoylcarnitine (C10:1) | Apolipoprotein H (beta-2-glycoprotein I) |
| Linoleoylcarnitine (C18:2) | Apolipoprotein N |
| Oleoylcarnitine (C18) | Arp2/3 complex 34 kDa subunit |
| Palmitoylcarnitine (C16) | Coagulation factor X |
| Stearoylcarnitine (C18) | Coagulation factor XI |
| Beta-muricholate | Coagulation factor XIII B chain |
| Cholate | Alpha-2 antiplasmin |
| Glycocholate | Immunoglobulin-2 |
| Taurochenodeoxycholate | Serum albumin |
| Taurocholate | Murinoglobulin-1 |
| Taurodeoxycholate | Haptoglobin |
| Coagulation factor XIII A chain | Alpha-1-macroglobulin |
| Complement C4 | Immunoglobulin-2 |
| Complement C5 | Fructosyllysine |
| Complement C8 gamma chain | Proteasome subunit alpha type-6 |
| Creatine kinase M-type | Serine (or cysteine) peptidase inhibitor |
| | clade C member 1 |
| Ig kappa chain C | Serpin family F member 2 |
| Beta-2-microglobulin | Thyroxine-binding globulin |
| Alpha-2-macroglobulin | Allantoic acid |
| Prothrombin | Biotinidase |
| Igh-6 protein | EGF-containing fibulin-like extracellular |
| | matrix protein 1 |
| Immunoglobulin J chain | Elongation factor 1-alpha |
| Kininogen | Ethylmalonate |
| Kininogen 1 | Extracellular matrix protein 1 |
| Carboxylesterase 1 | Fibronectin 1 |
| T-kininogen 2 | Fibulin-1 |
| Proteasome subunit beta type-10 | Fructose-bisphosphate aldolase |
| Protein Z-dependent protease inhibitor | Gelsolin |
| Serine (or cysteine) peptidase inhibitor | Glycosylphosphatidylinositol specific |
| clade B member 10 | phospholipase D1 |
| Serine (or cysteine) peptidase inhibitor | Keratin type II cytoskeletal 5 |
| clade B member 6a | |
| Serine protease inhibitor A3N | Myosin regulatory light chain RLC-A |
| 10 kDa heat shock protein, mitochondrial | Peptidyl-prolyl cis-trans isomerase A |
| 1-methyl-5-imidazoleacetate | Peptidyl-prolyl cis-trans isomerase |
| | FKBP1A |
| 5-methylthioadenosine (MTA) | Retinoic acid receptor responder 2 |
| Adenylate kinase 4, mitochondrial | Sacsin molecular chaperone |
| Alpha-soluble NSF attachment protein | Tmprss13 protein |
| C4b-binding protein beta chain | Actin-related protein 2/3 complex subunit |
| | 4 |
| Calpain small subunit 1 | Chenodeoxycholate |
| EH domain-containing protein 3 | Complement C1s subcomponent |
| Glucuronate | Complement C8 alpha chain |
| Integrin beta | Similar to immunoglobulin kappa-chain |
| | VK-1 |
| Integrin subunit alpha V | Pipecolate |
| Macrophage migration inhibitory factor | Proteasome subunit alpha type-3 |
| Microfibril-associated glycoprotein 4 | 5-methyl-2′-deoxycytidine |
| Myosin regulatory light polypeptide 9 | Alpha-actinin-1 |
| Palmitate (16:0) | Lysyl oxidase-like 1 |
| Peroxiredoxin 3 | |

In one embodiment, the molecular signature or profile of the invention is characteristic of grade S2 of the silent phase of Alzheimer's disease if the level, amount or concentration of at least 1 biomarker, preferably at least 2, 3, 4, 5, 6, 7, 8, 9, 10, 11, 12, 13, 14, 15, 16, 17, 18, 19, 20, 21, 22, 23, 24, 25, 26, 27, 28, or 29 biomarkers selected from the group of biomarkers of Table 5C is substantially similar (i.e., is no more than 5% lower or higher), when taking in comparison to the level, amount or concentration of the same biomarker(s) in a substantially healthy subject or a population of substantially healthy subjects.

TABLE 5C

| | |
|---|---|
| 14-3-3 protein beta/alpha | Ergothioneine |
| Apolipoprotein A-I | Oleate/vaccenate (18:1) |
| Acetylcarnitine (C2) | Polyubiquitin-C |
| Myristoylcarnitine (C14) | Actin-related protein 3 |
| Palmitoleoylcarnitine (C16:1) | Creatine kinase B-type |
| Deoxycholate | Igh-6 protein |
| Coagulation factor XII | Corticosteroid-binding globulin |
| Complement C4-like | Fibulin-5 |
| Vitamin K-dependent protein C | Major urinary protein |
| Ig gamma-2B chain C region | Mannan-binding lectin serine peptidase |
| | 2 |
| N⁶,N⁶,N⁶-trimethyllysine | Apolipoprotein B-100 |
| Proteasome subunit alpha type | Complement factor H-related protein |
| Proteasome subunit alpha type-7 | Serine protease inhibitor A3K |
| Proteasome subunit beta type-1 | Serine protease inhibitor A3L |
| Coactosin-like protein | |

In one embodiment, the molecular signature or profile of the invention is characteristic of grade S2 of the silent phase of Alzheimer's disease if: the level, amount or concentration of at least 1 biomarker, preferably at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or more biomarkers selected from the group of biomarkers of Table 5A is substantially lower (i.e., is more than 5% lower), when taking in comparison to the level, amount or concentration of the same biomarker(s) in a substantially healthy subject or a population of substantially healthy subjects, —the level, amount or concentration of at least 1 biomarker, preferably at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or more biomarkers selected from the group of biomarkers of Table 5B is substantially higher (i.e., is more than 5% higher), when taking in comparison to the level, amount or concentration of the same biomarker(s) in a substantially healthy subject or a population of substantially healthy subjects, and/or the level, amount or concentration of at least 1 biomarker, preferably at least 2, 3, 4, 5, 6, 7, 8, 9, 10, 11, 12, 13, 14, 15, 16, 17, 18, 19, 20, 21, 22, 23, 24, 25, 26, 27, 28, or 29 biomarkers selected from the group of biomarkers of Table 5C is substantially similar (i.e., is no more than 5% lower or higher), when taking in comparison to the level, amount or concentration of the same biomarker(s) in a substantially healthy subject or a population of substantially healthy subjects.

In one embodiment, the molecular signature or profile of the invention is characteristic of grade S3 of the silent phase of Alzheimer's disease if the level, amount or concentration of at least 1 biomarker, preferably at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or more biomarkers selected from the group of biomarkers of Table 6A is substantially lower (i.e., is more than 5% lower), when taking in comparison to the level, amount or concentration of the same biomarker(s) in a substantially healthy subject or a population of substantially healthy subjects.

TABLE 6A

| | |
|---|---|
| 14-3-3 protein epsilon | Glycocholate |
| 14-3-3 protein gamma | Taurodeoxycholate |
| 14-3-3 protein theta | Coagulation factor XIII A chain |
| Isobutyrylcarnitine (C4) | Complement C8 gamma chain |
| Isovalerylcarnitine (C5) | Creatine kinase M-type |
| Complement C2 | Ig kappa chain C |
| Alpha-2-macroglobulin | Beta-2-microglobulin |
| Complement factor properdin | Proteasome subunit beta type-10 |
| Ceruloplasmin | Protein Z-dependent protease inhibitor |
| Serum amyloid A protein | Serine protease inhibitor A3N |
| Proteasome subunit alpha type-1 | 10 kDa heat shock protein, mitochondrial |
| Proteasome subunit alpha type-2 | 5-methylthioadenosine (MTA) |
| Proteasome subunit alpha type-4 | Adenylate kinase 4, mitochondrial |
| 2,3-dihydroxyisovalerate | C4b-binding protein beta chain |
| 5-oxoproline | Calpain small subunit 1 |
| Calreticulin | Integrin beta |
| Carboxypeptidase B2 | Macrophage migration inhibitory factor |
| Chromogranin A | Microfibril-associated glycoprotein 4 |
| Clathrin heavy chain | Myosin regulatory light polypeptide 9 |
| FYN-binding protein 1 | Peroxiredoxin 3 |
| Hyaluronidase | Superoxide dismutase [Mn], mitochondrial |
| Malate dehydrogenase, cytoplasmic | Apolipoprotein C-I |
| Multiple inositol polyphosphate | Apolipoprotein C-II (predicted) |
| phosphatase 1 | |
| N-acetylasparagine | Apolipoprotein H (beta-2-glycoprotein I) |
| Ribonate (ribonolactone) | Apolipoprotein N |
| Sulfate | Coagulation factor X |
| Talin 2 | Coagulation factor XI |
| Apolipoprotein A-IV | Coagulation factor XIII B chain |
| Actin-related protein 2/3 complex subunit | Alpha-2 antiplasmin |
| 1B | |
| Carboxylesterase 1E | Murinoglobulin-2 |
| Propionylcarnitine (C3) | Serum albumin |
| Complement C7 | Murinoglobulin-1 |
| Angiotensinogen | Haptoglobin |
| 5-hydroxylysine | Serine (or cysteine) peptidase inhibitor |
| | clade C member 1 |
| Gamma-glutamyl-alpha-lysine | Serpin family F member 2 |
| Lysine | Biotinidase |
| N⁶-acetyllysine | Ethylmalonate |
| N⁶-methyllysine | Extracellular matrix protein 1 |
| N,N,N-trimethyl-5-aminovalerate | Fibronectin 1 |
| Alpha-1-antiproteinase | Fibulin-1 |
| Serine protease inhibitor A3M | Fructose-bisphosphate aldolase |
| Beta-hydroxyisovalerate | Gelsolin |
| Carnosine | Glycosylphosphatidylinositol specific |
| | phospholipase D1 |
| Dimethylarginine | Peptidyl-prolyl cis-trans isomerase A |
| Hydroxyproline | Retinoic acid receptor responder 2 |
| Lipase | Sacsin molecular chaperone |
| Lumican | Chenodeoxycholate |
| Prolylhydroxyproline | Complement C8 alpha chain |
| Retinol-binding protein | Pipecolate |
| Transthyretin | Proteasome subunit alpha type-3 |
| Urinary protein I | Lysyl oxidase-like 1 |
| Butyrylcarnitine (C4) | Acetylcarnitine (C2) |
| Anserine | Deoxycholate |
| 14-3-3 protein eta | Vitamin K-dependent protein C |
| 14-3-3 protein zeta/delta | Proteasome subunit alpha type |
| 14-3-3 protein zeta/delta | Proteasome subunit alpha type |
| Apolipoprotein M | Proteasome subunit alpha type-7 |
| Actin-related protein 2/3 complex subunit 3 | Coactosin-like protein |
| Arachidonoylcarnitine (C20:4) | Actin-related protein 3 |
| Carnitine | Creatine kinase B-type |
| Cis-4-decenoylcarnitine (C10:1) | Igh-6 protein |
| Palmitoylcarnitine (C16) | Fibulin-5 |
| Stearoylcarnitine (C18) | Major urinary protein |
| Beta-muricholate | Mannan-binding lectin serine peptidase 2 |
| Cholate | Apolipoprotein B-100 |

In one embodiment, the molecular signature or profile of the invention is characteristic of grade S3 of the silent phase of Alzheimer's disease if the level, amount or concentration of at least 1 biomarker, preferably at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or more biomarkers selected from the group of biomarkers of Table 6B is substantially higher (i.e., is more than 5% higher), when taking in comparison to the level, amount or concentration of the same biomarker(s) in a substantially healthy subject or a population of substantially healthy subjects.

TABLE 6B

| | |
|---|---|
| Apolipoprotein D | Serine protease inhibitor Kazal-type 3- |
| | like |
| Rat apolipoprotein E protein | Alpha-1B-glycoprotein |
| Laurylcarnitine (C12) | Apolipoprotein E |
| Coagulation factor VII | Taurochenodeoxycholate |
| Complement C1q subcomponent subunit | Taurocholate |
| A | |
| Complement C1q subcomponent subunit | Complement C4 |
| B | |
| Complement C1q subcomponent subunit | Complement C5 |
| C | |
| Complement C4B | Alpha-2-macroglobulin |
| Globin a2 | Prothrombin |
| Globin a4 | Igh-6 protein |
| Globin c2 | Immunoglobulin J chain |
| Globin c3 | Kininogen |
| Serine (or cysteine) proteinase inhibitor | Kininogen 1 |
| clade A member 4 | |
| Immunoglobulin heavy chain variable | Carboxylesterase 1 |
| region | |
| Ig heavy chain V region IR2 | T-kininogen 2 |
| Ig lambda-2 chain C region | Serine (or cysteine) peptidase inhibitor |
| | clade B member 6a |
| Ig delta chain C region | 1-methyl-5-imidazoleacetate |
| Ig gamma-2C chain C region | Alpha-soluble NSF attachment protein |
| Proteasome subunit beta | EH domain-containing protein 3 |
| Proteasome subunit beta type | Glucuronate |
| Serpin A11 | Integrin subunit alpha V |
| 2-hydroxy-3-methylvalerate | Palmitate (16:0) |
| Alpha-hydroxyisovalerate | Serum amyloid P-component |
| Anti-F4/80 kappa light chain variable | Apolipoprotein C-III |
| region | |
| BWK3 | Apolipoprotein C-IV |
| F-actin-capping protein subunit alpha-2 | Arp2/3 complex 34 kDa subunit |
| Fructose | Alpha-1-macroglobulin |
| HGF activator | Murinoglobulin-2 |
| Histidine-rich glycoprotein | Fructosyllysine |
| Interleukin 1 receptor accessory protein | Proteasome subunit alpha type-6 |
| Octadecanedioate (C18) | Allantoic acid |
| Palmitoleate (16:1n7) | Elongation factor I-alpha |
| Phenylalanine | Keratin type II cytoskeletal 5 |
| Rat MHC class I truncated cell surface | Myosin regulatory light chain RLC-A |
| antigen mRNA | |
| Thioredoxin | Peptidyl-prolyl cis-trans isomerase |
| | FKBP1A |
| Transferrin receptor protein 1 | Tmprss13 protein |
| Voltage-dependent anion-selective | Actin-related protein 2/3 complex subunit |
| channel protein 3 | 4 |
| Actin-related protein 2 | Complement C1s subcomponent |
| Complement C3 | 5-methyl-2′-deoxycytidine |
| IgG-2a protein | Myristoylcarnitine (C14) |
| Serum albumin | Palmitoylcarnitine (C16:1) |
| Igh-6 protein | Coagulation factor XII |
| Proteasome subunit beta type-3 | Complement C4-like |
| Heparin cofactor 2 | Ig gamma-2B chain C region |
| 3-methyl-2-oxovalerate | N⁶,N⁶,N⁶-trimethyllysine |
| 3-methyl-2-oxobutyrate | Ergothioneine |
| Mannose-binding protein A | Oleate/vaccenate (18:1) |
| Xaa-Pro aminopeptidase 2 | Polyubiquitin-C |
| Ig kappa chain V region S211 | Corticosteroid-binding globulin |
| Plasma protease C1 inhibitor | Complement factor H-related protein |

In one embodiment, the molecular signature or profile of the invention is characteristic of grade S3 of the silent phase of Alzheimer's disease if the level, amount or concentration of at least 1 biomarker, preferably at least 2, 3, 4, 5, 6, 7, 8, 9, 10, 11, 12, 13, 14, 15, 16, 17, 18, 19, 20, 21, 22, 23, 24, or 25 biomarkers selected from the group of biomarkers of Table 6C is substantially similar (i.e., is no more than 5% lower or higher), when taking in comparison to the level, amount or concentration of the same biomarker(s) in a substantially healthy subject or a population of substantially healthy subjects.

TABLE 6C

| | |
|---|---|
| Octanoylcarnitine (C8) | Oleoylcarnitine (C18) |
| Globin d1 | Serine (or cysteine) peptidase |
| | inhibitor clade B member 10 |
| Complement C6 | Apolipoprotein A-II |
| Ribulonate/xylulonate/lyxonate | Thyroxine-binding globulin |
| Actin-related protein 2/3 | EGF-containing fibulin-like |
| complex subunit 5 | extracellular matrix protein 1 |
| Carboxylesterase 1C | Similar to immunoglobulin kappa- |
| | chain VK-1 |
| Coagulation factor IX | Alpha-actinin-1 |
| Myoglobin | 14-3-3 protein beta/alpha |
| Serotransferrin | Apolipoprotein A-I |
| 2-methylbutyrylcarnitine (C5) | Proteasome subunit beta type-1 |
| Complement C1s subcomponent | Serine protease inhibitor A3K |
| Ighg protein | Serine protease inhibitor A3L |
| Linoleoylcarnitine (C18:2) | |

In one embodiment, the molecular signature or profile of the invention is characteristic of grade S3 of the silent phase of Alzheimer's disease if: the level, amount or concentration of at least 1 biomarker, preferably at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or more biomarkers selected from the group of biomarkers of Table 6A is substantially lower (i.e., is more than 5% lower), when taking in comparison to the level, amount or concentration of the same biomarker(s) in a substantially healthy subject or a population of substantially healthy subjects, the level, amount or concentration of at least 1 biomarker, preferably at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or more biomarkers selected from the group of biomarkers of Table 6B is substantially higher (i.e., is more than 5% higher), when taking in comparison to the level, amount or concentration of the same biomarker(s) in a substantially healthy subject or a population of substantially healthy subjects, and/or the level, amount or concentration of at least 1 biomarker, preferably at least 2, 3, 4, 5, 6, 7, 8, 9, 10, 11, 12, 13, 14, 15, 16, 17, 18, 19, 20, 21, 22, 23, 24, or 25 biomarkers selected from the group of biomarkers of Table 6C is substantially similar (i.e., is no more than 5% lower or higher), when taking in comparison to the level, amount or concentration of the same biomarker(s) in a substantially healthy subject or a population of substantially healthy subjects.

The present invention also relates to a method of diagnosing a silent phase of Alzheimer's disease in a subject in need thereof, using the molecular signatures or profiles of the invention.

The present invention also relates to a method of stratifying a silent phase of Alzheimer's disease in a subject into grades, preferably into SI, S2 or S3 grades, using the molecular signatures or profiles of the invention.

The present invention also relates to a method of prognosticating the progress of a silent phase of Alzheimer's disease in a subject, using the molecular signatures or profiles of the invention.

In one embodiment, the methods of the invention comprise a step of providing a sample from the subject.

The term “sample” as used herein generally refers to any sample which may be tested for expression levels of a biomarker, preferably of biomarkers selected from the group of biomarkers of Table 1A or of Table IB (or of Table 2A, Table 2B or Table 2C).

In one embodiment, the methods of the invention comprise a step of providing a sample from the subject.

In one embodiment, the sample is a body tissue or a bodily fluid sample.

In one embodiment, the sample is a body tissue sample. Examples of body tissues include, but are not limited to, muscle, nerve, brain, heart, lung, liver, pancreas, spleen, thymus, esophagus, stomach, intestine, kidney, testis, prostate, ovary, hair, skin, bone, breast, uterus, bladder and spinal cord.

In a preferred embodiment, the sample is not a body tissue sample.

In a preferred embodiment, the sample is a bodily fluid. Examples of bodily fluids include, but are not limited to, blood, plasma, serum, lymph, ascetic fluid, cystic fluid, urine, bile, nipple exudate, synovial fluid, bronchoalveolar lavage fluid, sputum, amniotic fluid, peritoneal fluid, cerebrospinal fluid, pleural fluid, pericardial fluid, semen, saliva, sweat, feces, stools, and alveolar macrophages.

In a preferred embodiment, the sample is a bodily fluid selected from the group comprising of consisting of blood, plasma and serum.

In a preferred embodiment, the sample is not a cerebrospinal fluid sample.

In a preferred embodiment, the sample is not feces or stools.

In one embodiment, the sample was previously taken from the subject, i.e., the methods of the invention do not comprise a step of recovering a sample from the subject. Consequently, according to this embodiment, the methods of the invention are non-invasive methods or “in vitro methods”.

In one embodiment, the methods of the invention comprise a step of determining the subject's molecular signature or profile according to the present invention in said sample from the subject.

In one embodiment, the step of determining the subject's molecular signature or profile comprises a sub step of measuring the levels, amounts or concentrations of at least 1 biomarker, preferably of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or more biomarkers selected from the group of biomarkers of Table 1A or of Table IB, as described hereinabove.

In one embodiment, the step of determining the subject's molecular signature or profile comprises a sub step of measuring the levels, amounts or concentrations of at least 1 biomarker, preferably of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or more biomarkers selected from the group of biomarkers of Table 2A, Table 2B or Table 2C, as described hereinabove.

In one embodiment, the levels, amounts or concentrations of biomarkers may be measured by methods well known in the art. Such method include, but are not limited to, mass spectrometry (such as, e.g, tandem mass spectrometry [MS/MS], chromatography-assisted mass spectrometry and combinations thereof), immunohistochemistry, multiplex methods (Luminex), western blot, enzyme-linked immunosorbent assay (ELISA), sandwich ELISA, fluorescent-linked immunosorbent assay (FLISA), enzyme immunoassay (EIA), radioimmunoassay (RIA), RT-PCR, RT-qPCR, Northern Blot, hybridization techniques (such as, e.g, use of microarrays, and combination thereof including but not limited to, hybridization of amplicons obtained by RT-PCR, sequencing such as, for example, next-generation DNA sequencing (NGS) or RNA-seq (also known as “whole transcriptome shotgun sequencing”)), and the like.

In one embodiment, the methods of the invention comprise a step of correlating the subject's molecular signature or profile with at least one reference signature or profile, as described hereinabove.

The reference signature or profile may be either implemented in the software or an overall median or other arithmetic mean across measurements may be built.

In one embodiment, the step of correlating the subject's molecular signature or profile with at least one reference signature or profile may be carried out by entering the subject's molecular signature or profile in an algorithm previously trained with levels, amounts or concentrations of biomarkers determined in reference subjects in order to decipher each of the reference signatures or profiles. The trained algorithm will compare the subject's molecular signature or profile with the reference signatures or profiles. In one embodiment, the algorithm returns a percentage of fitting of the subject's molecular signature or profile with each of the at least one reference signatures or profiles, preferably with each of the four reference signatures or profiles, i.e., healthy, grade SI, grade S2 and grade S3.

In one embodiment, if the subject's molecular signature or profile fits with healthy reference signature or profile, the subject is assigned as not suffering from a silent phase of Alzheimer's disease.

In one embodiment, if the subject's molecular signature or profile fits with either of the grade SI, grade S2 or grade S3 reference signature or profile, the subject is assigned as suffering from a silent phase of Alzheimer's disease, preferably from grade SI, grade S2 or grade S3 of the silent phase of Alzheimer's disease.

In one embodiment, if the subject's molecular signature or profile fits with either of the grade SI, grade S2 or grade S3 biomarker variation profile of Table 3, the subject is assigned as suffering from a silent phase of Alzheimer's disease, preferably from grade SI, grade S2 or grade S3 of the silent phase of Alzheimer's disease.

In one embodiment, if the subject's molecular signature or profile comprises: at least 1 biomarker, preferably at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or more biomarkers selected from the group of biomarkers of Table 4A which level, amount or concentration is substantially lower (i.e., is more than 5% lower), when taking in comparison to the level, amount or concentration of the same biomarker(s) in a substantially healthy subject or a population of substantially healthy subjects, at least 1 biomarker, preferably at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or more biomarkers selected from the group of biomarkers of Table 4B which level, amount or concentration is substantially higher (i.e., is more than 5% higher), when taking in comparison to the level, amount or concentration of the same biomarker(s) in a substantially healthy subject or a population of substantially healthy subjects, and/or—at least 1 biomarker, preferably at least 2, 3, 4, 5, 6, 7, 8, 9, 10, 11, 12, 13, 14, 15, 16, 17, 18, 19, 20, 21, 22, or 23 biomarkers selected from the group of biomarkers of Table 4C which level, amount or concentration is substantially similar (i.e., is no more than 5% lower or higher), when taking in comparison to the level, amount or concentration of the same biomarker(s) in a substantially healthy subject or a population of substantially healthy subjects, then the subject is assigned as suffering from grade SI of the silent phase of Alzheimer's disease.

In one embodiment, if the subject's molecular signature or profile comprises: at least 1 biomarker, preferably at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or more biomarkers selected from the group of biomarkers of Table 5A which level, amount or concentration is substantially lower (i.e., is more than 5% lower), when taking in comparison to the level, amount or concentration of the same biomarker(s) in a substantially healthy subject or a population of substantially healthy subjects, —at least 1 biomarker, preferably at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or more biomarkers selected from the group of biomarkers of Table 5B which level, amount or concentration is substantially higher (i.e., is more than 5% higher), when taking in comparison to the level, amount or concentration of the same biomarker(s) in a substantially healthy subject or a population of substantially healthy subjects, and/or at least 1 biomarker, preferably at least 2, 3, 4, 5, 6, 7, 8, 9, 10, 11, 12, 13, 14, 15, 16, 17, 18, 19, 20, 21, 22, 23, 24, 25, 26, 27, 28, or 29 biomarkers selected from the group of biomarkers of Table 5C which level, amount or concentration is substantially similar (i.e., is no more than 5% lower or higher), when taking in comparison to the level, amount or concentration of the same biomarker(s) in a substantially healthy subject or a population of substantially healthy subjects, then the subject is assigned as suffering from grade S2 of the silent phase of Alzheimer's disease.

In one embodiment, if the subject's molecular signature or profile comprises: —at least 1 biomarker, preferably at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or more biomarkers selected from the group of biomarkers of Table 6A which level, amount or concentration is substantially lower (i.e., is more than 5% lower), when taking in comparison to the level, amount or concentration of the same biomarker(s) in a substantially healthy subject or a population of substantially healthy subjects, at least 1 biomarker, preferably at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or more biomarkers selected from the group of biomarkers of Table 6B which level, amount or concentration is substantially higher (i.e., is more than 5% higher), when taking in comparison to the level, amount or concentration of the same biomarker(s) in a substantially healthy subject or a population of substantially healthy subjects, and/or at least 1 biomarker, preferably at least 2, 3, 4, 5, 6, 7, 8, 9, 10, 11, 12, 13, 14, 15, 16, 17, 18, 19, 20, 21, 22, 23, 24, or 25 biomarkers selected from the group of biomarkers of Table 6C which level, amount or concentration is substantially similar {i.e., is no more than 5% lower or higher), when taking in comparison to the level, amount or concentration of the same biomarker(s) in a substantially healthy subject or a population of substantially healthy subjects, then the subject is assigned as suffering from grade S3 of the silent phase of Alzheimer's disease.

In one embodiment, the correlation is associated to a fitting score for each of the four reference signatures or profiles, i.e., healthy, grade SI, grade S2 and grade S3, thereby allowing the secondary stratification.

In one embodiment, the methods of the invention comprise a step of diagnosing the subject as being affected with a silent phase of Alzheimer's disease, based on the correlation of the subject's individual signature or profile with the reference signatures or profiles.

In one embodiment, the methods of the invention comprise a step of stratifying the silent phase of Alzheimer's disease in the subject into grades, preferably into SI, S2 or S3 grades, based on the correlation of the subject's individual signature or profile with the reference signatures or profiles, such as, e.g, based on the correlation with the variations of level, amount or concentration of at least 1 biomarker, preferably at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or more biomarkers as shown in Table 3.

In one embodiment, the methods of the invention comprise a step of prognosticating the progress of a silent phase of Alzheimer's disease in the subject, based on the correlation of the subject's individual signature or profile with the reference signatures or profiles.

In one embodiment, the methods of the invention comprise a step of determining a personalized course of treatment for the subject, based on the correlation of the subject's individual signature or profile with the reference signature or profile.

The present invention also relates to a method of treating a subject affected with a silent phase of Alzheimer's disease, using the molecular signatures or profiles of the invention. The present invention also relates to a method of treating a subject affected with a silent phase of Alzheimer's disease, such as from SI, S2 or S3 grade of the silent phase of Alzheimer's disease, using the molecular signatures or profiles of the invention.

The present invention also relates to a method of determining a personalized course of treatment in a subject affected with a silent phase of Alzheimer's disease, using the molecular signatures or profiles of the invention. The present invention also relates to a method of determining a personalized course of treatment in a subject affected with a silent phase of Alzheimer's disease, such as from SI, S2 or S3 grade of the silent phase of Alzheimer's disease, using the molecular signatures or profiles of the invention.

The present invention also relates to a method of defining a clinical management for a subject affected with a silent phase of Alzheimer's disease, using the molecular signatures or profiles of the invention. The present invention also relates to a method of defining a clinical management for a subject affected with a silent phase of Alzheimer's disease, such as from SI, S2 or S3 grade of the silent phase of Alzheimer's disease, using the molecular signatures or profiles of the invention.

In one embodiment, the methods of treating or of determining a personalized course of treatment or of defining a clinical management comprise a step of diagnosing a silent phase of Alzheimer's disease in the subject as described hereinabove.

In one embodiment, the methods of treating or of determining a personalized course of treatment or of defining a clinical management comprise a step of stratifying the silent phase of Alzheimer's disease in the subject into grades, preferably into SI, S2 or S3 grades, as described hereinabove.

In one embodiment, the methods of treating or of determining a personalized course of treatment or of defining a clinical management comprise a further step of treating the subject.

In one embodiment, the step of treating the subject aims at preventing or reducing or alleviating the risks of developing clinical symptoms of Alzheimer's disease or dementia due to Alzheimer's disease in the subject.

Examples of treatments of the silent phase of Alzheimer's disease include, but are not limited to, beta-secretase 1 (Bacel) inhibitors, anti-amyloid antibodies, anti-inflammatory agents, anti-Tau antibodies, memory enhancers, synaptic plasticity enhancers, neuroprotection enhancers, microbiota modulators, inhibitors of the aggregation and seeding of Tau or Aβ, and anxiolytic drugs.

Examples of treatments of grade SI of the silent phase of Alzheimer's disease include, but are not limited to, beta-secretase 1 (Bacel) inhibitors, anti-amyloid antibodies, inhibitors of the seeding of Aβ, anti-inflammatory agents, and anxiolytic drugs.

Examples of treatments of grade S2 of the silent phase of Alzheimer's disease include, but are not limited to, beta-secretase 1 (Bacel) inhibitors, anti-amyloid antibodies, anti-inflammatory agents, anti-Tau antibodies, synaptic plasticity enhancers, neuroprotection enhancers, inhibitors of the aggregation and seeding of Tau or Aβ, memory enhancers, microbiota modulators, and anxiolytic drugs.

Examples of treatments of grade S3 of the silent phase of Alzheimer's disease include, but are not limited to, beta-secretase 1 (Bacel) inhibitors, anti-amyloid antibodies, anti-inflammatory agents, anti-Tau antibodies, memory enhancers, synaptic plasticity enhancers, neuroprotection enhancers, inhibitors of the aggregation and seeding of Tau or Aβ, and anxiolytic drugs.

Examples of Bacel inhibitors include, but are not limited to, CTS-21166 (CoMentis Inc.), verubecestat (MK-8931; Merck & Co., Inc.), solanezumab (Eli Lilly & Co.), lanabecestat (AZD3293; Astra7eneca and Eli Lilly & Co.), Elenbecestat (Biogen) and LY2886721 (Eli Lilly & Co.).

Examples of anti-amyloid antibodies include, but are not limited to, bapineuzumab (Janssen/Pfizer), solanezumab (Eli Lilly), crenezumab (Genentech), gantenerumab (Hoffman-La Roche), BAN2401 (Biogen/Eisai Inc.), GSK 933776 (GlaxoSmithKline), AAB-003 (Janssen/Pfizer), SAR228810 (Sanofi), BIIB037/BART (Biogen), ACI-24 (AC Immune) and aducanumab (Biogen/Eisai Inc.).

Examples of anti-inflammatory agents include, but are not limited to, non-steroidal anti-inflammatory drugs (NSAIDs), steroidal anti-inflammatory drugs (SAIDs), beta-agonists, anticholinergic agents, and methylxanthines.

Examples of anti-Tau antibodies include, but are not limited to, ABBV-8E12 (Abbvie), ACI-35 (AC Immune), BIIB092 (Biogen) and gosuranemab (Biogen).

Example of memory enhancers include, but are not limited to, metabolic substrates (e.g, glucose, ketones, supplemental oxygen), alkaloids (e.g, theobromine, caffeine), vitamins, amino acids, minerals, micronutrients, plant extracts and their derivatives, herbs or herbal nutritional supplements (e.g., ginkgo, ginseng root).

Example of inhibitors of the aggregation and seeding of Tau include, but are not limited to, TRx0237 (TauRx) and Morphomer Tau (AC Immune).

Examples of synaptic plasticity enhancers include, but are not limited to, blarcamesine (Anavex Life Sciences), CT1812 (Cognition Therapeutics), GRF6019 (Alkahest), and LM11A-31-BHS (Pharmatrophix).

Example of microbiota modulators include, but are not limited to, sodium oligomannate (Green Valley Pharmaceuticals), SLAB51, ProBiotic-4, and fecal microbiota transplantation (FMT) from substantially healthy subjects. For a review on microbiota modulators for the prevention or treatment of Alzheimer's disease, see Bonfili et al, 2020 (FEES J. Epub ahead of print).

Examples of neuroprotection enhancers include, but are not limited to, huperzine A, nefiracetam, propentofylline, rivastigmine and SGS-742.

Examples of anxiolytic drugs include, but are not limited to, 5-HT1AR agonists (such as, e.g, buspirone, gepirone, and tandospirone), GABAA receptor positive allosteric modulators (GABAAR PAMS) (such as, e.g, adinazolam, alprazolam, bromazepam, camazepam, chlordiazepoxide, clobazam, clonazepam, clorazepate, clotiazepam, cloxazolam, diazepam, ethyl loflazepate, etizolam, fludiazepam, halazepam, ketazolam, lorazepam, medazepam, nordazepam, oxazepam, pinazepam, prazepam, alpidem, phenobarbital, carisoprodol, meprobamate, chlormezanone, ethanol (alcohol), etifoxine, imepitoin, kava, skullcap, and valerian), a2d voltage-dependent calcium channel (VDCC) blockers (such as, e.g, gabapentin, gabapentin enacarbil, phenibut and pregabalin), antidepressants (such as, e.g, escitalopram, duloxetine, trazodone, clomipramine, mirtazapine, phenelzine, agomelatine, bupropion, tianeptine, vilazodone, and vortioxetine), sympatholytics (such as, e.g, prazosin, clonidine, dexmedetomidine, guanfacine, and propranolol), benzoctamine, cannabidiol, cycloserine, fabomotizole, hydroxyzine, kanna, lavender, lorpiprazole, mebicar, mepiprazole, nicotine, opipramol, oxaflozane, phenaglycodol, phenibut, picamilon, selank, tiagabine, tofisopam and validolum.

For a review of the pipeline of drugs and biologies in clinical trials (as of 2020) for the treatment of Alzheimer's disease, see Cummings et al, 2020 {Alzheimers Dement (NY). 6(1):e12050), which lists 121 agents currently in clinical trials. The content of Cummings et al, 2020 is incorporated by reference, in particular the drugs and biologies recited in FIG. 1 and in Tables 1, 2, 3 and 4.

In one particular embodiment, the methods of treating or of determining a personalized course of treatment or of defining a clinical management comprise a step of administering at least one beta-secretase 1 (Bacel) inhibitor, anti-amyloid antibody, anti-inflammatory agent, anti-Tau antibody, memory enhancer, synaptic plasticity enhancer, neuroprotection enhancer, microbiota modulator, inhibitor of the aggregation and seeding of Tau orAβ, or anxiolytic drug—as defined hereinabove—to the subject diagnosed with a silent phase of Alzheimer's disease, such as, e.g., an asymptomatic phase or a prodromal phase of Alzheimer's disease.

In one particular embodiment, the methods of treating or of determining a personalized course of treatment or of defining a clinical management comprise a step of administering at least one anti-amyloid antibody—as defined hereinabove—to the subject diagnosed with a silent phase of Alzheimer's disease, such as, e.g., an asymptomatic phase or a prodromal phase of Alzheimer's disease.

In one particular embodiment, the methods of treating or of determining a personalized course of treatment or of defining a clinical management comprise a step of administering at least one anti-amyloid antibody selected from the group comprising or consisting of bapineuzumab, solanezumab, crenezumab, gantenerumab, BAN2401, GSK 933776, AAB-003, SAR228810, BIIB037/BART, ACI-24 and aducanumab, to the subject diagnosed with a silent phase of Alzheimer's disease, such as, e.g, an asymptomatic phase or a prodromal phase of Alzheimer's disease.

In one particular embodiment, the methods of treating or of determining a personalized course of treatment or of defining a clinical management comprise a step of administering aducanumab to the subject diagnosed with a silent phase of Alzheimer's disease, such as, e.g, an asymptomatic phase or a prodromal phase of Alzheimer's disease.

In one particular embodiment, the methods of treating or of determining a personalized course of treatment or of defining a clinical management comprise a step of administering gantenerumab to the subject diagnosed with a silent phase of Alzheimer's disease, such as, e.g, an asymptomatic phase or a prodromal phase of Alzheimer's disease.

In one particular embodiment, the methods of treating or of determining a personalized course of treatment or of defining a clinical management comprise a step of administering oligomannate to the subject diagnosed with a silent phase of Alzheimer's disease, such as, e.g, an asymptomatic phase or a prodromal phase of Alzheimer's disease.

In one embodiment, the step of treating the subject aims at preventing or reducing or alleviating the risks of cardiovascular diseases associated with Alzheimer's disease.

Cardiovascular diseases are known to be factors contributing to the development or increased risk of developing Alzheimer's disease. Hence, preventing or reducing or alleviating the risks of cardiovascular diseases may be a secondary preventive measure to prevent or reduce or alleviate the risks of developing clinical symptoms of Alzheimer's disease or dementia due to Alzheimer's disease in the subject.

Means and methods for preventing or reducing or alleviating the risks of cardiovascular diseases are known in the art, and include, without limitation, stopping smoking, keeping alcohol to a minimum, eating a healthy and balanced diet, exercising for at least 150 minutes per week, controlling blood pressure, taking regular health tests, treating diabetes if applicable, and the like.

In one embodiment, the step of treating the subject aims at slowing down the risks of cognitive decline associated with Alzheimer's disease.

Cognitive decline is known to be a factor contributing to the development or increased risk of developing clinical symptoms of Alzheimer's disease or dementia due to Alzheimer's disease in the subject.

Means and methods for slowing down the risks of cognitive decline are well known in the art, and include, without limitation, reading, learning foreign languages, playing musical instruments, and maintaining an active social life (such as, by volunteering in a local community, taking part in group sports, trying new activities or hobbies), and the like.

In one embodiment, the step of treating the subject aims at treating or alleviating factors associated with Alzheimer's disease.

Factors associated with Alzheimer's disease are known in the art, and include, without limitation, hearing loss, depression, loneliness or social isolation, exacerbated sedentary lifestyle, and the like.

The present invention also relates to a method of recruiting a subject affected with a silent phase of Alzheimer's disease in a clinical trial, using the molecular signatures or profiles of the invention. The present invention also relates to a method for selecting a subject affected with a silent phase of Alzheimer's disease for enrollment in a clinical trial, using the molecular signatures or profiles of the invention.

The present invention also relates to a method of recruiting a subject affected with a silent phase of Alzheimer's disease, such as from SI, S2 or S3 grade of the silent phase of Alzheimer's disease, in a clinical trial, using the molecular signatures or profiles of the invention. The present invention also relates to a method for selecting a subject affected with a silent phase of Alzheimer's disease, such as from SI, S2 or S3 grade of the silent phase of Alzheimer's disease, for enrollment in a clinical trial, using the molecular signatures or profiles of the invention.

In one embodiment, the methods of recruiting a subject in a clinical trial or selecting a subject for enrollment in a clinical trial comprise a step of diagnosing a silent phase of Alzheimer's disease in the subject as described hereinabove.

In one embodiment, the methods of recruiting a subject in a clinical trial or selecting a subject for enrollment in a clinical trial comprise a step of stratifying the silent phase of Alzheimer's disease in the subject into grades, preferably into SI, S2 or S3 grades, as described hereinabove.

In one embodiment, the methods of recruiting a subject in a clinical trial or selecting a subject for enrollment in a clinical trial comprise a further step of recruiting the subject in the clinical trial or selecting the subject for enrollment in the clinical trial.

In one embodiment, the clinical trial involves treatment of a silent phase of Alzheimer's disease. In one embodiment, the clinical trial involves investigation of the safety and/or efficacy of a treatment of the silent phase of Alzheimer's disease.

In one embodiment, optionally during the clinical trial, the methods of the invention could be implemented at least one more time or at least two more times, for example during the clinical trial and/or at the end of the clinical trial to monitor the molecular signatures or profiles of the invention while the subject is treated with the test compound.

Alternatively, the methods of the invention could also be used at the end of a clinical trial as Primary or Secondary Outcome Measures: change From Baseline in one or more biomarkers of the molecular signatures or profiles of the invention.

In one embodiment, the subject is an animal, preferably a mammal.

Examples of mammals include, but are not limited to, humans, non-human primates (such as, e.g, chimpanzees, and other apes and monkey species), farm animals (such as, e.g, cattle, horses, sheep, goats, and swine), domestic animals (such as, e.g, rabbits, dogs, and cats), laboratory animals (such as, e.g, rats, mice and guinea pigs), and the like. The term does not denote a particular age or gender, unless explicitly stated otherwise.

In one embodiment, the subject is a primate, including human and non-human primates.

In one embodiment, the subject is a human.

In one embodiment, the subject is a man or a woman.

In one embodiment, the subject is a child. In one embodiment, the subject is an adult.

In one embodiment, the subject is at risk of developing Alzheimer's disease. Risk factors of Alzheimer's disease include, but are not limited to, age, family history, heredity, and others.

Age is the greatest known factor of Alzheimer's disease. Most subjects with symptomatic

Alzheimer's disease are 65 and older. After age 65, the risk of Alzheimer's disease doubles every five years. After age 85, the risk reaches nearly one-third.

Therefore, in one embodiment, the subject is above 20 years old. In one embodiment, the subject is above 30 years old. In one embodiment, the subject is above 40 years old. In one embodiment, the subject is above 50 years old. In one embodiment, the subject is above 60 years old. In one embodiment, the subject is above 70 years old. In one embodiment, the subject is above 80 years old.

In one embodiment, the subject is aged from 0 to 20 years old. In one embodiment, the subject is aged from 20 to 40 years old. In one embodiment, the subject is aged from 40 to 50 years old. In one embodiment, the subject is aged from 50 to 55 years old. In one embodiment, the subject is aged from 55 to 60 years old. In one embodiment, the subject is aged from 60 to 65 years old. In one embodiment, the subject is aged from 65 to 70 years old. In one embodiment, the subject is aged from 70 to 75 years old. In one embodiment, the subject is aged from 75 to 80 years old. In one embodiment, the subject is aged from 80 to 85 years old. In one embodiment, the subject is aged from 85 to 90 years old.

Family history is another risk factor of Alzheimer's disease.

Therefore, in one embodiment, the subject has a relative, preferably a parent, a grandparent, a great grandparent, a brother, a sister, an aunt, an uncle, a niece, a nephew, or a first cousin who has been diagnosed with or identified as having Alzheimer's disease. Heredity is another risk factor of Alzheimer's disease. Studies have shown that single nucleotide polymorphism (SNP) in some loci may influence the risk of Alzheimer's disease. See, e.g., Jansen et al, 2019. Nat Genet. 51(3): 404-413.

Therefore, in one embodiment, the subject has at least one single nucleotide polymorphism (SNP) in at least one locus selected from those defined in Table 1 of Jansen et al, 2019, which is incorporated by reference.

Other risks factors of Alzheimer's disease are known. These include, without limitation, Down syndrome, sleep deprivation, head injuries, heart diseases, diabetes, stroke, high blood pressure, hypercholesternlemic.

The present invention also relates to a computer system for diagnosing a silent phase of Alzheimer's disease in a subject in need thereof, using the molecular signatures of the invention. The present invention also related to a computer-implemented method for diagnosing a silent phase of Alzheimer's disease in a subject, using the molecular signatures of the invention.

The present invention also relates to a computer system for stratifying a silent phase of Alzheimer's disease in a subject into grades, preferably into SI, S2 or S3 grades, using the molecular signatures of the invention. The present invention also related to a computer-implemented method for stratifying a silent phase of Alzheimer's disease in a subject into grades, preferably into SI, S2 or S3 grades, using the molecular signatures of the invention.

The present invention also relates to a computer system for prognosticating the progress of a silent phase of Alzheimer's disease in a subject, using the molecular signatures of the invention. The present invention also related to a computer-implemented method for prognosticating the progress of a silent phase of Alzheimer's disease in a subject, using the molecular signatures of the invention.

The present invention also relates to a computer system for determining a personalized course of treatment in a subject affected with a silent phase of Alzheimer's disease, using the molecular signatures of the invention. The present invention also related to a computer-implemented method for determining a personalized course of treatment in a subject affected with a silent phase of Alzheimer's disease, using the molecular signatures of the invention.

As used herein, the term “computer system” refers to any and all devices capable of storing and processing information and/or capable of using the stored information to control the behavior or execution of the device itself, regardless of whether such devices are electronic, mechanical, logical, or virtual in nature. The term “computer system” can refer to a single computer, but also to a plurality of computers working together to perform the function described as being performed on or by a computer system. A method implemented using a computer system is referred to as a “computer-implemented method”.

In one embodiment, the computer system according to the present invention comprises:

(i) at least one processor, and

(ii) at least one computer-readable storage medium that stores code readable by the processor.

As used herein, the term “processor” is meant to include any integrated circuit or other electronic device capable of performing an operation on at least one instruction word, such as, e.g, executing instructions, codes, computer programs, and scripts which it accesses from a storage medium. However, the term “processor” should not be construed to be restricted to hardware capable of executing software, and refers in a general way to a processing device, which can for example include a computer, a microprocessor, an integrated circuit, or a programmable logic device (PLD). The processor may also encompass one or more graphics processing units (GPU), whether exploited for computer graphics and image processing or other functions. Additionally, the instructions and/or data enabling to perform associated and/or resulting functionalities may be stored on any processor-readable medium, including, but not limited to, an integrated circuit, a hard disk, a magnetic tape (including floppy disk and zip diskette), an optical disc (including Blu-ray, compact disc and digital versatile disc), a flash memory (including memory card and USB flash drive) a random-access memory (RAM) (including dynamic and static RAM), a read-only memory (ROM) or a cache. Instructions may be in particular stored in hardware, software, firmware or in any combination thereof.

Examples of processors include, but are not limited to, central processing units (CPU), microprocessors, digital signal processors (DSPs), general purpose microprocessors, application specific integrated circuits (ASICs), field programmable logic arrays (FPGAs), and other equivalent integrated or discrete logic circuitry.

The present invention also related to a computer program comprising software code readable by the processor adapted to perform, when executed by said processor, the computer-implemented methods as described herein.

The present invention also relates to a computer-readable storage medium comprising code readable by the processor which, when executed by said processor, causes the processor to carry out the steps of the computer-implemented methods as described herein.

Examples of computer-readable storage medium include, but are not limited to, an integrated circuit, a hard disk, a magnetic tape (including floppy disk and zip diskette), an optical disc (including Blu-ray, compact disc and digital versatile disc), a flash memory (including memory card and USB flash drive) a random-access memory (RAM) (including dynamic and static RAM), a read-only memory (ROM) or a cache.

In one embodiment, the computer-readable storage medium is a non-transitory computer-readable storage medium.

In one embodiment, the code stored on the computer-readable storage medium, when executed by the processor of the computer system, causes the processor to:

a. receive an input level, amount or concentration of at least five biomarkers selected from Table 1A or Table IB determined in a sample previously obtained from the subject,

b. analyze and transform the input level, amount or concentration of the at least five biomarkers by organizing and/or modifying each input level to derive a probability score and/or a classification label via at least one machine learning algorithm,

c. generate an output, wherein the output is the classification label and/or the probability score, and

d. provide a diagnosis of the subject as being affected or not with a silent phase of Alzheimer's disease based on the output; or provide a stratification of the silent phase of Alzheimer's disease in the subject into grades, preferably into SI, S2 or S3 grades based on the output; or

provide a prognosis of the progress of a silent phase of Alzheimer's disease based on the output; or

provide a personalized course or information to determine a personalized course of treatment for the subject based on the output.

In one embodiment, the code stored on the computer-readable storage medium, when executed by the processor of the computer system, causes the processor to:

a. receive an input level, amount or concentration of at least five biomarkers selected from Table 2A, Table 2B or Table 2C determined in a sample previously obtained from the subject,

b. analyze and transform the input level, amount or concentration of the at least five biomarkers by organizing and/or modifying each input level to derive a probability score and/or a classification label via at least one machine learning algorithm,

c. generate an output, wherein the output is the classification label and/or the probability score, and

d. provide a diagnosis of the subject as being affected or not with a silent phase of Alzheimer's disease based on the output; or

provide a stratification of the silent phase of Alzheimer's disease in the subject into grades, preferably into SI, S2 or S3 grades based on the output; or

provide a prognosis of the progress of a silent phase of Alzheimer's disease based on the output; or

provide a personalized course or information to determine a personalized course of treatment for the subject based on the output.

As used herein, the terms “learning algorithm” or “machine learning algorithm” refer to computer-executed algorithms that automate analytical model building, e.g, for clustering, classification or profile recognition. Learning algorithms perform analyses on training datasets provided to the algorithm. Learning algorithms output a “model”, also referred to as a “classifier”, “classification algorithm” or “diagnostic algorithm”. Models receive, as input, test data and produce, as output, an inference or a classification of the input data as belonging to one or another class, cluster group or position on a scale, such as diagnosis, stage, prognosis, disease progression, responsiveness to a drug, etc.

“Datasets” are collections of data used to build a machine learning mathematical model, so as to make data-driven predictions or decisions. In “supervised learning” (i.e., inferring functions from known input-output examples in the form of labelled training data), three types of machine learning datasets are typically dedicated to three respective kinds of tasks: “training”, i.e., fitting the parameters; “validation”, i.e., tuning machine learning hyperparameters (which are parameters used to control the learning process); and “testing”, i.e., checking independently of a training dataset exploited for building a mathematical model that the latter model provides satisfying results.

A variety of learning algorithms can be used to infer a condition or state of a subject. Machine learning algorithms may be supervised or unsupervised. Learning algorithms include, but are not limited to, artificial neural networks (e.g, back propagation networks), discriminant analyses (e.g, Bayesian classifier, Fischer analysis), support vector machines, decision trees (e.g, recursive partitioning processes, such as classification and regression trees [CART]), random forests, linear classifiers (e.g, multiple linear regression [MLR], partial least squares [PLS] regression, principal components regression [PCR]), hierarchical clustering and cluster analysis. The learning algorithm generates a model or classifier that can be used to make an inference, e.g, an inference about a disease state of a subject.

In one embodiment, the at least one machine learning algorithm was previously trained with at least one training dataset.

In one embodiment, the at least one training dataset comprises information relating to the level, amount or concentration of the same at least five biomarkers of Table 1A (as the at least five biomarkers of step a. of the computer-implemented method) from samples previously obtained from reference subjects (i.e., from subjects of known Alzheimer's disease status).

In one embodiment, the at least one training dataset comprises information relating to the level, amount or concentration of the same at least five biomarkers of Table 1A from samples previously obtained from substantially healthy subject and from subjects known to be affected with a silent stage of Alzheimer's disease.

In one embodiment, the training dataset comprises the biomarker variation profile of Table 3.

In one embodiment, the at least one machine learning algorithm is selected from the group comprising an artificial neural network (ANN), a perceptron algorithm, a deep neural network, a clustering algorithm, a k-nearest neighbors algorithm (k-NN), a decision tree algorithm, a random forest algorithm, a linear regression algorithm, a logistic regression algorithm, a linear discriminant analysis (LDA) algorithm, a quadratic discriminant analysis (QDA) algorithm, a support vector machine (SVM), a Bayes algorithm, a simple rule algorithm, a clustering algorithm, a meta-classifier algorithm, a Gaussian mixture model (GMM) algorithm, a nearest centroid algorithm, a gradient boosting algorithm (such as, e.g, an extreme gradient boosting [XG Boost] algorithm or an adaptative boosting [AdaBoost] algorithm), a linear mixed effects model algorithm, and a combination thereof.

### BRIEF DESCRIPTION OF THE DRAWINGS

FIG. 1. Alzheimer's silent phase stratification in grades SI, S2 and S3 in function of cerebral main events: soluble Aβ peptide production, hyperphosphorylation of Tau and appearance of aggregated lesion (senile plaques and tangles). Onset of dementia in a subject indicates the start of the so-called clinical phase.

FIG. 2. Comparison between Alzheimer's progression in humans, transgenic mice and AgenT's rats. As figured, transgenic mice are not suitable to reproduce the AD progression as observed in humans, especially its silent phase. By contrast, characteristics of the AgenT's rat model make it a closer model of the silent phase of AD than transgenic animals. All these features make the AgenT rat model a powerful tool to better predict blood biomarker behavior according to the stage of progression. This model thus constitutes a suitable study system to characterize new biomarkers or panel of biomarkers for the development of an early diagnosis.

FIG. 3. Amyloid cerebral imaging doesn't constitute a powerful approach to detect subjects with Alzheimer's. Indeed, 30% of AD patients are PIB-PET (positron emission tomography [PET] utilizing Pittsburgh compound B [PIB]) Aβ and 40% of healthy individuals are PIB-PET Aβ+. This strongly reduces its pertinence as a diagnosis.

FIG. 4. Clinical validation of AgenT Grade S3 plasma variations. To decipher the clinical pertinence of plasmatic variations that we observed in the AgenT rats, we correlated the variations observed in diagnosed patients (meta-analysis based on Doecke et al, 2012. Arch Neurol. 69(10): 1318-25; Map stone et al, 2014. Nat Med. 20(4):415-8; Olazaran et al., 2015. J Alzheimers Dis. 45(4): 1157-73; Kim et al., 2017. J Alzheimers Dis. 60(3):809-817) and those observed in the grade S3 rats. We observed that 75% of already-described variations are also present in AgenT rats (***p<0.0001; ***r2=0.71). This result strongly confirms the high level of clinical pertinence of the plasma variation observed in the AgenT grade S3 rats.

FIG. 5. Clinical validation of AgenT Grade SI plasma variation. To decipher the clinical pertinence of plasmatic variations that we observed in the AgenT rats, we correlated the variations observed in young Down syndrome individuals (Caracausi et al, 2018. Sci Rep. 8(1):2977) and those observed in the grade SI rats. We observed that 74% of already-described variations are also present in AgenT rats (***p<0.0001; ***r2=0.76). This result strongly confirms the high level of clinical pertinence of the plasma variation observed in the AgenT grade SI rats.

FIG. 6. Example of blood biomarker variation during Alzheimer's progression. Blood biomarkers evolve through the pathology progression in a non-linear fashion. It is thus impossible to presume the variations during the silent phase based only on the variations from AD-diagnosed patients. Illustration of three typical examples is shown in this figure (alpha-2-macroglobulin, 5-hydroxylysine and ethylmalonate). Points on the curves indicated with (1) indicate variations observed in plasma of AgenT rats, assessed by mass spectrometry.

FIG. 7. Blood biomarkers identification process. The 119 “best-in-class” blood biomarkers suitable to detect AD silent phase subjects sounds to be an innovative strategy combining neuroscience and artificial intelligence.

FIG. 8. Scientific literature suspected the diagnosis pertinence of some of the identified blood biomarkers. However, their silent AD profile was yet unknown, in particular their specific, non-linear variations all along the silent phase, which could not have been deciphered from the available preclinical or clinical data. Our approach leads therefore to an understanding of the biomarkers' evolution over time, in the silent phase of AD, and with a high level of confidence (ApoE, serpin A1 and complement C3). Points on the curves indicated with (1) indicate variations observed in plasma of AgenT rats, assessed by mass spectrometry; points on the curves indicated with (2) indicate variations observed in plasma of Alzheimer's diagnosed patients (adapted from Thambisetty et al, 2011. PLoS One. 6(12):e28527); points on the curves indicated with (3) indicate variations observed in plasma of Alzheimer's diagnosed patients (adapted from Wang et al, 2014. PLoS One. 9(2):e89041); points on the curves indicated with (4) indicate variations observed in plasma of Alzheimer's diagnosed patients (adapted from Liao et al, 2007. Proteomics Clin Appl. 1(5): 506-12).

FIG. 9. Comparison between brain-released biomarkers and biomarkers produced by peripheral organs. Measuring biomarkers released from peripheral organs in “amyloid stress conditions” hugely increases the specificity (i.e., the true positive rate) and the sensitivity (i.e., the true negative rate) of the test.

FIGS. 10A-B. Example of neural network based on 14 biomarkers randomly chosen in the biomarkers of Table 1A for diagnosing a silent stage of Alzheimer's disease in a subject. The list of biomarkers is the following: 10 kDa heat shock protein, mitochondrial; 5-hydroxylysine (from the biomarker family “Lysine and conjugates”); adenylate kinase 4, mitochondrial; calreticulin; creatine kinase B-type (from the biomarker family “Creatinine kinase family”); ergothioneine; fructosyllysine (from the biomarker family “Lysine and conjugates”); globin c2 (from the biomarker family “Globin family”); integrin subunit alpha V; myoglobin (from the biomarker family “Globin family”); peptidyl-prolyl cis-trans isomerase FKBP1A; retinoic acid receptor responder 2; Tmprss13 protein; and transferrin receptor protein 1.

FIG. 10A. Neural network structure trained to identify AD status and stratification. In this illustrative example, the neural network comprises 14 inputs on the left side (i.e., the 14 biomarkers randomly chosen in the biomarkers of Table 1A) and 4 outputs on the right side (i.e., the four profiles healthy, grade SI, grade S2 and grade S3).

FIG. 10B. Accuracy of the trained neural network for the silent AD detection over 5 cross-validations.

FIGS. 11A-B. Example of neural network based on 14 biomarkers randomly chosen in the biomarkers of Table 1A for stratifying a silent phase of Alzheimer's disease in a subject into different grades of the silent phase. The 14 biomarkers are the same as described in FIG. 10.

FIG. 11A. Stratification method exemplified for two samples (A and B). The method comprises the steps of measuring the level, amount or concentration of biomarkers; processing raw data in a trained neural network to compare the subject's signature or profile with each of the reference signatures or profiles (healthy, grade SI, grade S2 and grade S3); calculating a fitting score; and stratifying the subject according to its profile.

FIG. 11B. Ad hoc confusion matrix of the trained neural network for the silent AD stratification over 5 cross-validations.

FIG. 12. Experimental design used to validate the 119 best-in-class biomarkers in humans by transfer learning.

FIG. 13. Average accuracy for 2, 5, 15 and 25 randomly selected biomarkers of Table 1A or non-Table 1A constituents. Analysis realized with 250 random selections using two-way ANOVA.

FIG. 14. Average accuracy for 2, 5, 15 and 25 randomly selected biomarkers of Table 1A or non-Table 1A constituents. Analysis realized with 1000 random selections using Mann Whitney's nonparametric test.

FIG. 15A-C. Performances obtained with 1000 random selections using two-way ANOVA.

FIG. 15A. Percentage of accuracy for 2, 5, 15 and 25 randomly selected biomarkers of Table 1A or non-Table 1A constituents.

FIG. 15B. Percentage of biomarker combination with an accuracy over 70%.

FIG. 15C. Average accuracy for 2, 5, 15 and 25 randomly selected biomarkers of Table 1A or non-Table 1A constituents.

### EXAMPLES

The present invention is further illustrated by the following examples.

### Example 1

Material & Methods

Animal

The AgenT rat model (U.S. Pat. No. 10,159,227; European patent EP3066203) was induced through injection of adeno-associated viruses (AAV) coding for human mutant APP (double-mutant APP751 cDNA containing the Swedish and London mutations) and presenilin 1 (PS1) (cDNA containing the M146L mutation (pENTR4-PS 1-SI 82M146L)) genes into the hippocampi of adult rodents (8-week-old Wistar male rats).

Controls rats were injected with AAV coding for presenilin-1 (PS1) alone.

This disruptive technology has allowed the localized production of exogenous APP and PS1 mutated proteins in a small number of neurons. These neurons produce Aβ42 peptide which progressively diffuses throughout the hippocampal tissue. The majority of the hippocampal cells thus have no genetic modification, making it a relevant model for non-genetic forms of the disease that represent more than 92% of cases (Prince et al, 2015. World Alzheimer Report 2015. The global impact of dementia: An analysis of prevalence, incidence, cost and trends (Rep.). London: Alzheimer's disease international (ADI)).

The pathophysiological relevance of this model has been validated by comparing it to post-mortem samples of AD patients. The concentration of Aβ42 peptide gradually increases to reach, at the late stage, concentrations comparable to those measured in the hippocampus of AD patients. As hyper-phosphorylation of the endogenous Tau protein gradually takes place, the memory capacity simultaneously declines, reproducing the chronology of events progression seen in clinics. Amyloid plaques and cerebral amyloid angiopathy develop only in aged AgenT rats. Intraneuronal aggregates of hyperphosphorylated Tau protein confirm a full commitment of the Tau pathology (Audrain et al., 2018. Cereb Cortex. 28(11):3976-3993).

Plasma Extraction

To identify plasmatic biomarkers, bloods were sampled from 33 controls rats and 33 AgenT rats.

The sampling age has been performed to obtain:

16 controls rats aged 1 to 3 months post injection (Grade SI),

16 AgenT rats aged 1 to 3 months post injection (Grade SI),

10 controls rats aged 8 to 10 months post injection (Grade S2),

10 AgenT rats aged 8 to 10 months post injection (Grade S2),

7 controls rats aged 15 to 30 months post injection (Grade S3), and

7 AgenT rats aged 15 to 30 months post injection (Grade S3).

In order to avoid batch effect, these experiments were based on 6 independent rat cohorts.

Each of the blood samples were associated with a specific grade of progression (SI, S2, S3) corresponding to the different neurological disorders. This stratification makes it possible to characterize the evolution of the deregulated molecules according to the disease progression.

EDTA plasma was obtained through cardiac puncture after centrifugation at 2,000×g for 10 minutes and was aliquoted into 0.5 mL polypropylene tubes and stored at −80° C.

Quantification of Plasma Constituents by Mass Spectrometry

Proteomic Mass Spectrometry

Plasma samples were shipped frozen on dry ice. 5 μL of sample were denatured, reduced and alkylated using Biognosys' Denature and Reduction/Alkylation Buffers for 30 minutes at 37° C.

Subsequently, 80 μg of protein was digested using 1.6 μg of trypsin (Promega) per sample overnight at 37° C. Peptides were desalted using C18 MacroSpincolumns (The Nest Group) according to the manufacturer's instructions and dried down using a Speed Vac system.

Peptides were resuspended in 22 μL of LC solvent A (1% acetonitrile in water with 0.1% formic acid) and spiked with Biognosys' iRT kit calibration peptides prior to mass spectrometric analyses.

Peptide concentrations were determined using microBCA (Thermo Fisher) and UV/Vis spectrometer (SPECTROstar Nano, BMG Labtech).

For data-independent acquisition (DIA) liquid chromatography tandem-mass spectrometry (LC-MS/MS) measurements, 5 μg of peptides per sample were injected to a C18 column (CSH-C18 1.7 pm, 300 pm inner diameter, 150 mm length) on a Waters M-Class LC connected to a Thermo Scientific Fusion Lumos Tribrid mass spectrometer equipped with a next generation nanoFlex electrospray source.

LC solvents were:

LC solvent A: 1% acetonitrile in water with 0.1% formic acid;

LC solvent B: 15% water in acetonitrile with 0.1% formic acid.

The nonlinear LC gradient was 1-49% solvent B in 40 minutes, followed by steps of 90% B for 1 minute and 1% B for 4 minutes.

A DIA method with one full range survey scan and 29 DIA windows was used.

HRM mass spectrometric data were analyzed using Spectronaut Pulsar X software (Biognosys). The false discovery rate on protein and peptide level was set to 1%, data was filtered using row-based extraction. The assay library (protein inventory) generated in this project was used for the analysis. The HRM measurements analyzed with Spectronaut were normalized using local regression normalization (Callister et al, 2006 J Proteome Res. 5(2):277-86).

Distance in heat maps was calculated using the “Manhattan” method, the clustering using “ward.D” for both axes.

Principal component analysis was conducted in R using prcomp and a modified ggbiplot function for plotting, and partial least squares discriminant analysis was performed using mixOMICSpackage.

General plotting was done in R using ggplot2 package.

Metabolomic Mass Spectrometry

Samples were prepared using the automated MicroLab STAR® system from Hamilton Company. Several recovery standards were added prior to the first step in the extraction process for quality control purposes.

Samples were extracted with methanol under vigorous shaking for 2 minutes (Glen Mills GenoGrinder 2000) to precipitate proteins and dissociate small molecules bound to proteins or trapped in the precipitated protein matrix, followed by centrifugation to recover chemically diverse metabolites.

The resulting extract was divided into five fractions:

two for analysis by two separate reverse phase (RP)/ultra-performance liquid chromatography (UPLC)-MS/MS methods using positive ion mode electrospray ionization (ESI),

one for analysis by RP/UPLC -MS/MS using negative ion mode ESI,
one for analysis by hydrophilic interaction liquid chromatography (HILIC)/UPLC-MS/MS using negative ion mode ESI, and one kept for backup.

Samples were placed briefly on a TurboVap® (Zymark) to remove the organic solvent. The sample extracts were stored overnight under nitrogen before preparation for analysis. All methods utilize a Waters ACQUITY UPLC and a Thermo Scientific Q-Exactive high resolution/accurate mass spectrometer interfaced with a heated electrospray ionization (HESI-II) source and Orbitrap mass analyzer operated at 35,000 mass resolution.

The sample extract was dried, then reconstituted in solvents compatible to each of the four methods. Each reconstitution solvent contained a series of standards at fixed concentrations to ensure injection and chromatographic consistency.

One aliquot was analyzed using acidic positive ion conditions, chromatographically optimized for more hydrophilic compounds. In this method, the extract was gradient-eluted from a C18 column (Waters UPLC BEHC18-2.1×100 mm, 1.7 pm) using water and methanol, containing 0.05% perfluoropentanoic acid (PFPA) and 0.1% formic acid (FA).

A second aliquot was also analyzed using acidic positive ion conditions, but was chromatographically optimized for more hydrophobic compounds. In this method, the extract was gradient-eluted from the aforementioned C18 column using methanol, acetonitrile, water, 0.05% PFPA and 0.01% FA, and was operated at an overall higher organic content.

A third aliquot was analyzed using basic negative ion optimized conditions using a separate dedicated C18 column. The basic extracts were gradient-eluted from the column using methanol and water, however with 6.5 mM ammonium bicarbonate pH 8.

The fourth aliquot was analyzed via negative ionization following elution from a HILIC column (Waters UPLC BEH Amide 2.1×150 mm, 1.7 pm) using a gradient comprising water and acetonitrile with 10 mM ammonium formate pH 10.8.

The MS analysis alternated between MS and data-dependent MSn scans using dynamic exclusion. The scan range varied slightly between methods, but covered approximately 70-1000 m/z. Raw data were extracted, peak-identified, and quality control-processed using hardware and software. Compounds were identified by comparison to library entries of purified standards or recurrent unknown entities. Mass spectrometry facility maintains a library based on authenticated standards that contains the retention time/index (RI), mass to charge ratio (m/z), and chromatographic data (including MS/MS spectral data) of all molecules present in the library. Furthermore, biochemical identifications are based on three criteria: retention index within a narrow RI window of the proposed identification, accurate mass match to the library +/−10 ppm, and the MS/MS forward and reverse scores. MS/MS scores are based on a comparison of the ions present in the experimental spectrum to ions present in the library entry spectrum. While there may be similarities between these molecules based on one of these factors, the use of all three data points can be utilized to distinguish and differentiate biochemicals. More than 4500 commercially available purified standard compounds have been acquired and registered into LIMS for analysis on all platforms for determination of their analytical characteristics. A variety of curation procedures are performed to ensure that a high-quality dataset is made available for statistical analysis and data interpretation. The quality control and curation processes are designed to ensure accurate and consistent identification of true chemical entities, and to remove those representing system artifacts, mis-assignments, redundancy, and background noise. Data analysts use visualization and interpretation software to confirm the consistency of peak identification among the various samples. Library matches for each compound are checked for each sample and corrected if necessary. Peaks are quantified as area-under-the-curve detector ion counts. For studies spanning multiple days, a data adjustment step is performed to correct block variation resulting from instrument inter-day tuning differences, while preserving intra day variance. Essentially, each compound is corrected in balanced run-day blocks by registering the daily medians to equal one (1.00), and adjusting each data point proportionately (termed the “block correction”). For studies that do not require more than one day of analysis, no adjustment of raw data is necessary, other than scaling for purposes of data visualization.

Identification of Plasma Biomarkers

The starting point for this analysis was to exclude irrelevant biomarkers. To do this, we gradually carried out the three following steps:

(1) We removed all biomarkers whose variance didn't meet some threshold, i.e., biomarkers that had almost the same value in all samples;

(2) We ran several linear clustering algorithms (linear SVM, gradient tree boosting, random forest, logistic regression, etc.), which gave a relative importance to biomarkers, and we excluded those of negligible importance. We could consider that, at the end of this step, all grossly irrelevant biomarkers were discarded.

(3) We then performed different recursive feature eliminations (RFE) with cross-validation using several algorithms that assign weights to features on the remaining biomarkers, and we finally selected those biomarkers. In more details, an RFE is a feature selection method that fits a model and removes recursively the weakest biomarkers until a relevant number of features is reached.

Once the most relevant biomarkers were identified, we selected the most informative ones for the silent phase of AD as follows:

(1) We recursively tested by cross-validation all possible combinations of these biomarkers with two different machine learning algorithms (a multilayer perceptron and a support-vector machine with a third-degree polynomial kernel). Thus, we successively found the best combinations of n biomarkers, with n ranging from 1 to 250.

(2) Among the combinations of biomarkers that have allowed us to obtain the best average score for cross-validation prediction, we chose the ones with the least number of biomarkers in order to avoid overfitting as much as possible.

With this last analysis, we obtained a list of 119 biomarkers (or biomarker families) that can be considered as the most characteristic of the different grades of AD.

Results

Silent AD Stratification

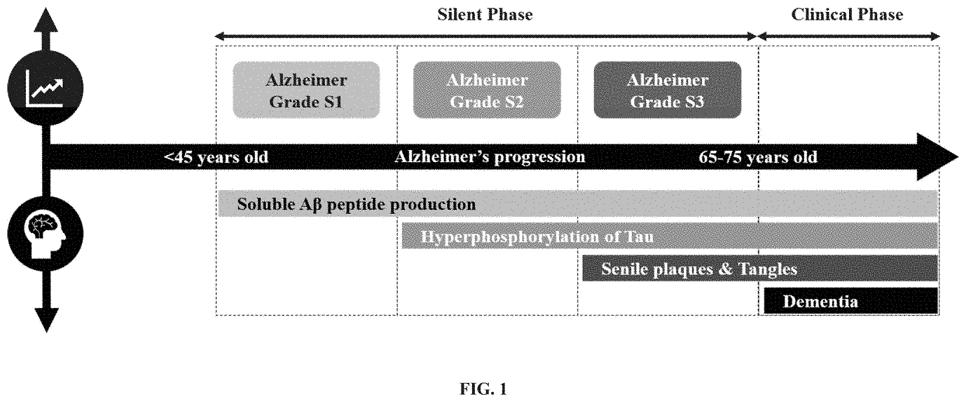

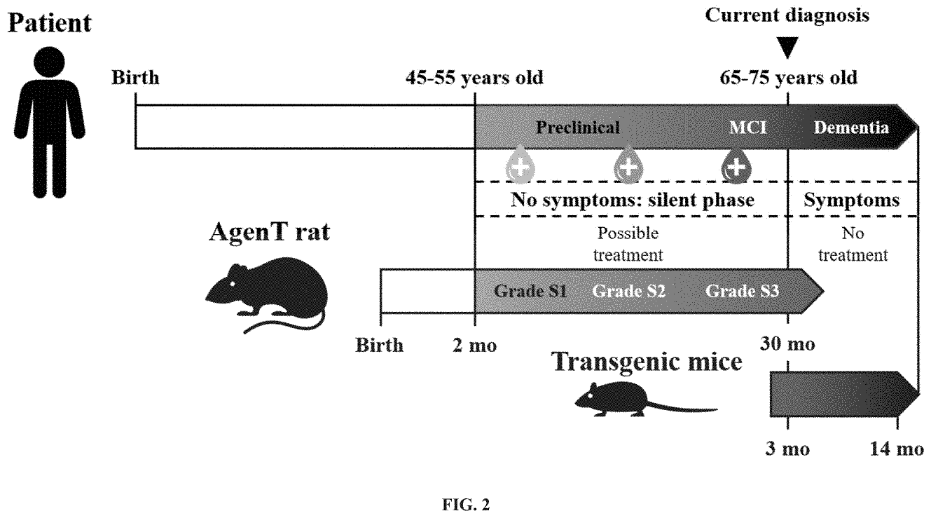

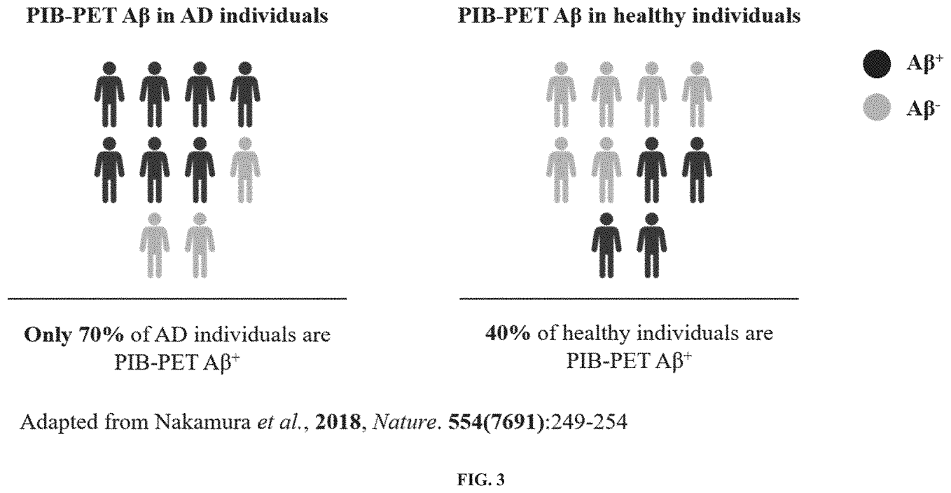

By combining longitudinal behavior and brain biochemistry analysis in the AgenT rats, we stratified the silent phase of AD according to 3 grades (FIG. 1 & Table 7).

TABLE 7
THREE GRADES OF THE SILENT PHASE OF
AD: CEREBRAL MODIFICATIONS AND LESION,
COGNITIVE IMPAIRMENT AND BEHAVIOR.

| | | Cerebral | Cognitive | |
|---|---|---|---|---|
| Grades | Cerebral modifications | lesions | impairments | Behavior |
| Healthy | | | | |
| Grade | Soluble Aβ₄₂ | | | Anxiety- |
| S1 | concentration | | | like |
| | dysregulation | | | syndrome |
| Grade | Accumulation of soluble | | Accelerated | Anxiety- |
| S2 | Aβ₄₂ peptides | | forgetting | like |
| | Hyperphosphorylation of | | | syndrome |
| | Tau | | | |
| Grade | Accumulation of soluble | Senile | Memory | Anxiety- |
| S3 | Aβ₄₂ peptides | plaques | impairments | like |
| | Increase of | Tangles | | syndrome |
| | hyperphosphorylated Tau | | | |

Grade SI is defined by a production of soluble Aβ42 in the cerebral tissue, in sufficient concentration to induce anxiety-like disorders.

Grade S2 is then defined by the accumulation of Aβ42 in the cerebral tissue in sufficient concentration to induce pathological hyperphosphorylation of tau epitopes and to promote an accelerating long-term forgetting.

Grade S3 is finally defined by an aggregation of both amyloid peptides (senile plaques) and phospho-Tau (tangles).

The silent phase stratification appears as the success key for biomarker identification and, by this way, to permit the development of a diagnosis of the silent phase of AD.

Determination of the Global Profile of Plasmatic Constituents

We carried out a global analysis thanks to mass spectrometry analysis. Proteomic, lipidomic and metabolomic approaches have been realized in order to identify the specific profile for each rat plasma sample, according to their grade of progression.

Thus, 2400 constituents have been measured. We generated a complete dataset by linking the plasmatic profile and the actual stage of progression (grade SI, S2 or S3) to start the identification of suitable blood biomarkers.

AsenT Rats Shown High Clinical Pertinence

A metanalysis of 4 published papers (Doecke et al., 2012. Arch Neurol. 69(10): 1318-25;

Map stone et al., 2014. Nat Med. 20(4):415-8; Olazaran et al., 2015. J Alzheimers Dis. 45(4): 1157-73; Kim et al., 2017. J Alzheimers Dis. 60(3):809-817) led to the identification of 90 deregulated molecules in the plasma of diagnosed AD patients.

Among these 90 molecules, 45 appeared as measured during the mass spectrometry assays in grade S3 AgenT rats.

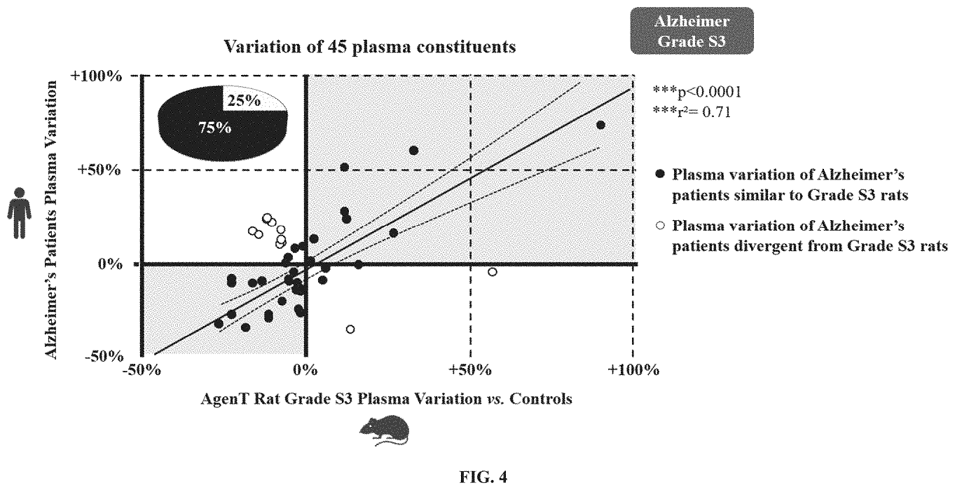

To decipher the clinical pertinence of plasmatic variations that we observed in the AgenT rats, we correlated the variations observed in diagnosed patients and those observed in the grade S3 rats. We observed that 75% of already-described variations are also present in AgenT rats (***p<0.0001; ***r2=0.71). This result strongly confirms the high level of clinical pertinence of the plasma variation observed in the AgenT grade S3 rats (FIG. 4 & Table 8).

TABLE 8
PLASMATIC VARIATIONS OF ALREADY-DESCRIBED
BIOMARKERS ON DIAGNOSED PATIENTS.

| | | AD/ | Grade |
|---|---|---|---|
| Prior art | Biomarkers | ctrl (%) | S3/Ctrl (%) |
| Doecke\| | Albumin | −5.0 | −3.67 |
| et al., 2012 | Alpha-1-antitrypsin | 11.0 | −7.40 |
| | Apolipoprotein E (ApoE) | −3.0 | 6.06 |
| | Cortisol | 28.0 | 11.86 |
| | Mean cell hemoglobin | −1.0 | 16.16 |
| | concentration | | |
| | Hemoglobin | −5.0 | 57.18 |
| | Tissue inhibitor of | 18.0 | −7.59 |
| | metalloproteinase 1 | | |
| | Insulin-like factor binding | 61.0 | 33.12 |
| | protein 2 | | |
| | Microglobulin | 24.0 | 12.43 |
| | Superoxide dismutase | 16.0 | 26.85 |
| Kim et al., | Cer16:0 | 10.0 | −7.92 |
| 2017 | Cer18:0 | 22.0 | −10.43 |
| | Cer20:0 | 1.0 | 1.47 |
| | Cer22:0 | 0.0 | −6.20 |
| | Cer24:0 | 3.0 | −5.34 |
| | Cer24:1 | 9.0 | −0.88 |
| | PC36:5 | −30.0 | −11.37 |
| | PC38:6 | −28.0 | −22.83 |
| | PC40:6 | −27.0 | −1.75 |
| Mapstone | PC38:6 | −10.9 | −22.83 |
| et al., 2014 | PC36:6 | −10.9 | −16.33 |
| | PC38:0 | −9.0 | −5.33 |
| | PC40:6 | −15.4 | −1.75 |
| | LysoPC18:2 | −9.4 | 5.11 |
| Olazarán | Glycodeoxycholic acid | 83.1 | −55.00 |
| et al., 2015 | TAG56:8 | −32.9 | −26.69 |
| | PC38:6 | −8.6 | −22.83 |
| | TAG56:7 | −35.0 | −18.50 |
| | PE38:5 | 17.2 | −16.28 |
| | Alanine | 15.3 | −14.48 |
| | PCO36:4 | −9.8 | −13.43 |
| | Glycine | 24.1 | −12.07 |
| | PE36:4 | 24.7 | −11.80 |
| | PC36:5 | −27.9 | −11.37 |
| | Arginine | 12.5 | −7.55 |
| | PC38:5 | −20.8 | −7.33 |
| | PE40:6e | −9.8 | −5.11 |
| | Asparagine | 7.9 | −3.40 |
| | PC37:6 | −14.6 | −2.91 |
| | PC40:5 | −10.8 | −2.66 |
| | Aspartic acid | −25.0 | −2.27 |
| | PC40:6 | −14.0 | −1.75 |
| | Methionine | 12.9 | 2.49 |
| | Cortisol | 52.0 | 11.86 |
| | Glutamic acid | −36.0 | 13.59 |
| | Deoxycholic acid | 74.8 | 90.39 |

AD/ctrl: variation observed in the cited reference between diagnosed AD sample and control sample (in %).
Grade S3/Ctrl: variation observed in the AgenT rats between AD grade S3 samples and control samples (in %).
Cer: ceramide; PC: phosphatidylcholine; PCO: alkyl ether -substituted phosphatidylcholine; LysoPC: lysophophatidylcholine; TAG: triacylglycerol; PE: phosphatidylethanolamine.

Epidemiological evidences suggest that by the age of 40, all individuals with Down syndrome (DS) have AD neuropathology (Lott & Head, 2005. Neurobiol Aging. 26(3):383-9). The complete penetrance of AD in DS individuals is due to the extra copy of the amyloid precursor protein (APP) gene caused by trisomy of chromosome 21 (Rovelet-Lecrux et al, 2006. Nat Genet. 38(1):24-6; Sleegers et al, 2006. Brain. 129(Pt 11):2977-83). This genetic predisposition leads to AD silent phase onset from birth in individuals with DS. Therefore, we consider that individuals with DS fastly convert in AD grade SI in their earliest life.

The analysis of the only—to the Inventor's knowledge—published paper of plasmatic biomarker variations in young DS individuals (Caracausi et al., 2018. Sci Rep. 8(1):2977) led to the identification of 46 deregulated molecules.

Among these molecules, 23 appeared as measured during the mass spectrometry assays in grades SI AgenT rats.

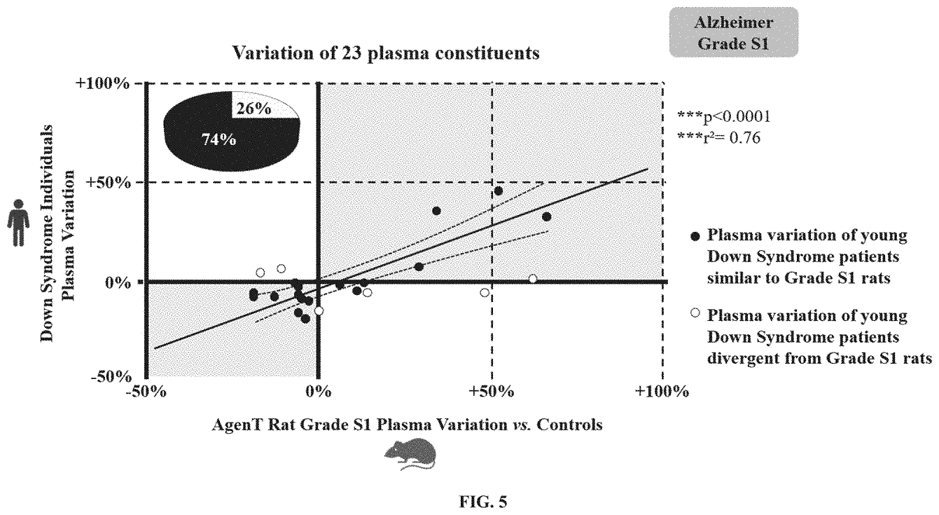

To decipher the clinical pertinence of plasmatic variations that we observed in the AgenT rats, we correlated the variations observed in young DS individuals and those observed in the grade SI rats. We observed that 74% of already-described variations are also present in AgenT rats (***p<0.0001; ***r2=0.76). This result strongly confirms the high level of clinical pertinence of the plasma variation observed in the AgenT grade SI rats (FIG. 5 & Table 9).

TABLE 9
PLASMATIC VARIATIONS OF ALREADY-DESCRIBED
BIOMARKERS IN DS SUBJECTS.

| | | DS/ | Grade |
|---|---|---|---|
| Prior art | Biomarkers | ctrl (%) | S1/Ctrl (%) |
| Caracausi | 2-hydroxybutyrate | −18 | −8 |
| et al., 2018 | 3-hydroxybutyrate | −4 | −9 |
| | Alanine | −10 | 6 |
| | Citrate | 14 | −1 |
| | Creatine | 63 | 1 |
| | Creatinine | −18 | −6 |
| | Fumarate | 53 | 45 |
| | Glucose | −5 | −3 |
| | Glutamate | 15 | −6 |
| | Glutamine | 7 | −2 |
| | Glycerol | 67 | 32 |
| | Glycine | 1 | −15 |
| | Histidine | −6 | −1 |
| | Isoleucine | −12 | −8 |
| | Lactate | 30 | 7 |
| | Leucine | −5 | −7 |
| | Mannose | −3 | −19 |
| | Methionine | 12 | −5 |
| | Phenylalanine | −2 | −10 |
| | Pyruvate | 35 | 35 |
| | Succinate | 49 | −6 |
| | Succinate | 49 | −6 |
| | Threonine | −5 | −16 |
| | Tyrosine | −16 | 4 |

DS/ctrl: variation observed in the cited reference between DS sample and control sample (in %).
Grade Sl/Ctrl: variation observed in the AgenT rats between AD grade SI samples and control samples (in %).

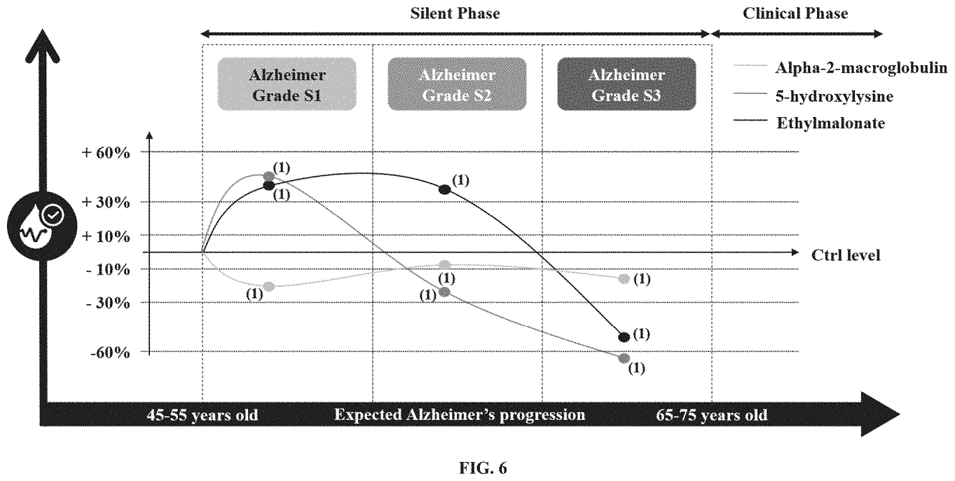

Interestingly, blood biomarkers evolve through the pathology progression. It is thus impossible to presume the variations during the silent phase based only on the variations from AD-diagnosed patients. Three typical examples (alpha-2-macroglobulin, 5-hydroxylysine and ethylmalonate) of this are shown in FIG. 6.

Identification of suitable plasma biomarkers for silent phase of Alzheimer's disease

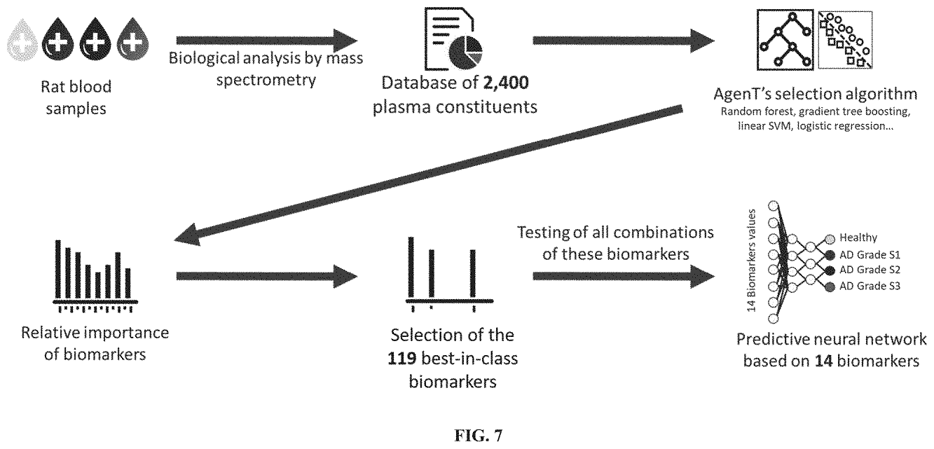

Once the clinical pertinence of the AgenT rat confirmed, we identified the best-in-class biomarkers suitable to detect silent AD using artificial intelligence approaches (FIG. 7).

We identified 119 best-in-class plasmatic biomarkers (biomarker families) suitable to detect the AD silent phase (Table 1A).

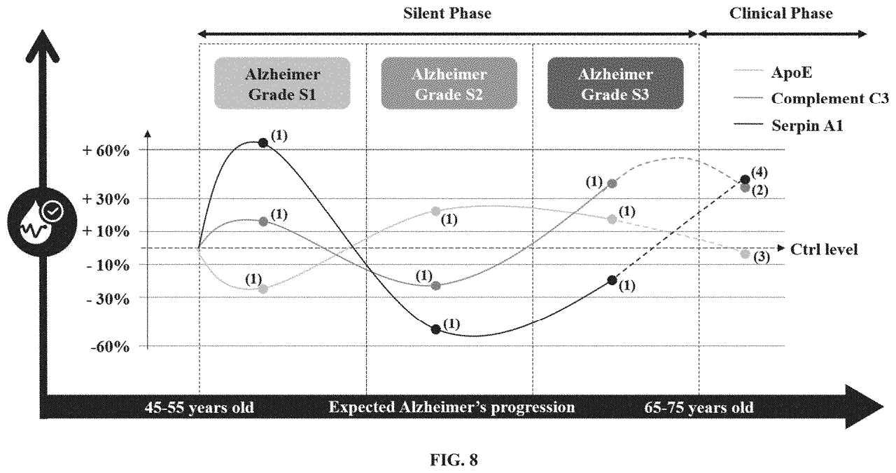

Interestingly, some biomarkers identified had already been suspected as potential AD biomarkers. However, their silent AD profile was yet unknown, in particular their specific, non-linear variations all along the silent phase (FIG. 8), which could not have been deciphered from the available preclinical or clinical data.

Our approach leads therefore to an understanding of the biomarkers' evolution over time, in the silent phase of AD, and with a high level of confidence.

Among the “biomarker families” of Table 1A, the following cluster proteins:

14-3-3 family: 14-3-3 proteins are a family of conserved regulatory molecules that are expressed in all eukaryotic cells. 14-3-3 proteins have the ability to bind a multitude of functionally diverse signaling proteins, including kinases, phosphatases, and transmembrane receptors. More than 200 signaling proteins have been reported as 14-3-3 ligands. The main 13-3-3 family members are: 14-3-3 protein beta/alpha, 14-3-3 protein epsilon, 14-3-3 protein eta, 14-3-3 protein gamma, 14-3-3 protein theta, 14-3-3 protein zeta/delta.

Arp2/3 complex proteins: Arp2/3 complex is a seven-subunit protein complex that plays a major role in the regulation of the actin cytoskeleton. It is a major component of the actin cytoskeleton and is found in most actin cytoskeleton-containing eukaryotic cells. The main Arp2/3 complex proteins are: Actin-related protein 2, Actin-related protein 2/3 complex subunit IB, Actin-related protein 2/3 complex subunit 3, Actin-related protein 2/3 complex subunit 4, Actin-related protein 2/3 complex subunit 5, Actin-related protein 3, Arp2/3 complex 34 kDa subunit.

Apolipoproteins: Apolipoproteins are proteins that bind lipids (oil-soluble substances such as fat and cholesterol) to form lipoproteins. They transport lipids (and fat-soluble vitamins) in blood, cerebrospinal fluid and lymph. The main apolipoproteins are: Apolipoprotein A-I, Apolipoprotein A-II, Apolipoprotein A-IV, Apolipoprotein B-100, Apolipoprotein C-I, Apolipoprotein C-II, Apolipoprotein C-III, Apolipoprotein C-IV, Apolipoprotein D, Apolipoprotein E, Apolipoprotein H, Apolipoprotein M, Apolipoprotein N.

Coagulation factor family: Coagulation factors are proteins in the blood that help control bleeding.

Complement system family: The complement system is a part of the immune system that enhances the ability of antibodies and phagocytic cells to clear microbes and damaged cells from an organism, promote inflammation, and attack the pathogen's cell membrane. It is part of the innate immune system, which is not adaptable and does not change during an individual's lifetime. The complement system can, however, be recruited and brought into action by antibodies generated by the adaptive immune system.

Globin family: The globins are a superfamily of heme-containing globular proteins, involved in binding and/or transporting oxygen.

Globulin family: The globulins are a family of globular proteins that have the higher molecular weights than albumins and are insoluble in pure water but dissolve in dilute salt solutions. Some globulins are produced in the liver, while others are made by the immune system. Globulins, albumins, and fibrinogen are the major blood proteins.

Kininogen family: Kininogens are proteins that are defined by their role as precursors for kinins, but that also can have additional roles. Kinins are biologically active peptides, the parent form is bradykinin. The main kininogens are: Kininogen, Kininogen 1, T -kininogen 2.

Proteasome complex family: The proteasome is a cylindrical complex containing a “core” of four stacked rings forming a central pore. Each ring is composed of seven individual proteins. The inner two rings are made of seven b subunits and the outer two rings each contain seven a subunits.

Serpin superfamily: Serpins are a superfamily of proteins with similar structures that were identified for their protease inhibition activity.

Other “biomarker families” of Table 1A cluster metabolites:

Lysine and derivates: Lysine plays several roles in humans, most importantly proteinogenesis, but also in the crosslinking of collagen polypeptides, uptake of essential mineral nutrients, and in the production of carnitine, which is key in fatty acid metabolism.

Carnitine and derivates: Carnitine is a conditionally essential nutrient that plays a vital role in energy production and fatty acid metabolism. Carnitine not obtained from food is synthesized endogenously from two essential amino acids, lysine and methionine. Aberrations in carnitine regulation are implicated in complications of diabetes mellitus, hemodialysis, trauma, malnutrition, cardiomyopathy, obesity, fasting, drug interactions, endocrine imbalances and other disorders (Flanagan et al, 2010. Role of carnitine in disease).

Choi ate and derivates: Cholic acid, also known as 3a,7a,12a-trihydroxy-5P-cholan-24-oic acid is a primary bile acid that is insoluble in water. Salts of cholic acid are called cholates. Cholic acid, along with chenodeoxycholic acid, is one of the two major bile acids produced by the liver, where it is synthesized from cholesterol. These two major bile acids are roughly equal in concentration in humans. Derivatives are made from cholyl-CoA, which exchanges its CoA with either glycine, or taurine, yielding glycocholic and taurocholic acid, respectively.

Valerate and derivates: A valerate compound is a salt or ester of valeric acid. It is also known as pentanoate. Many steroid-based pharmaceuticals, for example ones based on betamethasone or hydrocortisone, include the steroid as the valerate ester.

Peripheral biomarkers are more relevant than brain-released ones to predict the individual AD status.

Currently, all blood biomarkers under development are based on brain-released biomarkers, and in particular on Aβ42 peptides, Tau or phospho-Tau, growth factors, neuroinflammation players or neuronal cell death markers (e.g, neurofilament light chain (NfL)). This type of biomarkers suffers many limitations, strongly reducing their ability to detect asymptomatic AD patients.

Ab42 peptides are poorly correlated to the AD status. Indeed, for the same concentration of soluble Aβ42 peptides in the brain, one individual will develop AD but another one will not. This is the consequence of the individual sensitivity to “amyloid stress”. Without taking into account this individual sensitivity, it is impossible to detect silent AD with accuracy.

For blood Tau, phospho-Tau, growth factors, neuroinflammation players or neuronal cell death markers, although they could be of interest to improve the current clinical AD diagnosis, their late deregulation reduces their use to detect the asymptomatic patients.

To counteract these problems, using peripheral blood biomarkers appears as the best solution. Measuring biomarkers released from peripheral organs in “amyloid stress conditions” hugely increases the specificity (i.e., the true positive rate) and the sensitivity (i.e., the true negative rate) of the test.

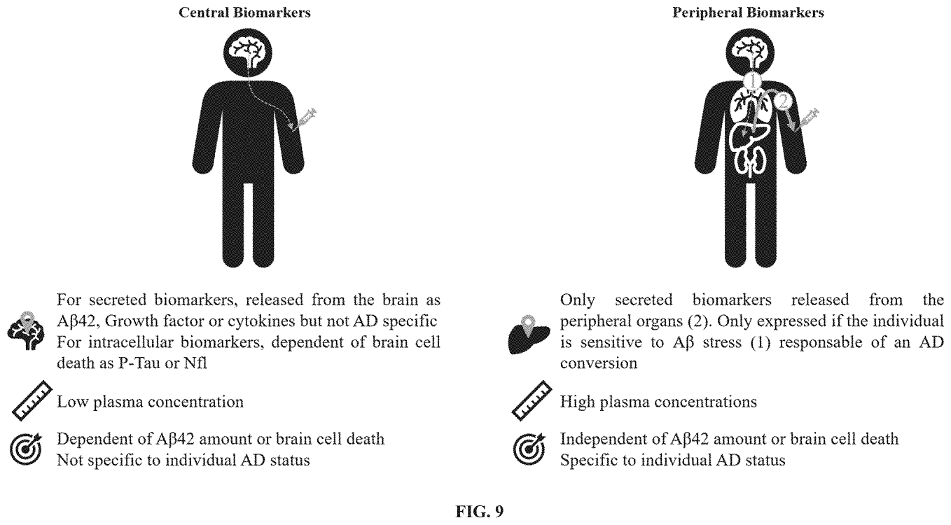

Indeed, only individuals under “amyloid stress” and responsive to this will develop AD and will present deregulated blood peripheral biomarkers. Main of the biomarkers identified therein are peripheral ones (FIG. 9).

Based-Biomarkers Predictive Neuronal Networks with a High Level of Accuracy

By taking a few biomarkers set at random from the list of 119 best-in-class plasmatic biomarkers identified, it is possible to train a neuronal network with reference subjects (training set) in order to define the 4 reference profiles as described previously. Using this trained neuronal network, it could be possible to calculate its accuracy using a new batch of subjects (test set not used to train the algorithm) or by cross validation techniques. The performances obtained were above 75%, using an artificial neural network. These performances were calculated for the ability of the trained algorithm to segregate the healthy subjects from the silent Alzheimer's subjects.

Taking a subset of 5 (Table 10A), 6 (Table 10B), 14 (Table IOC) and 26 (Table 10D) randomly selected from the biomarkers of Table 1A, and a feed forward neural network—more precisely a multilayer perceptron—with a logistic activation function, we could obtain an accuracy for silent AD detection over 5 cross-validations of over 75%.

TABLE 10A
EXAMPLE OF PERFORMANCES TO PREDICT
ALZHEIMER'S SILENT PHASE THANKS
TO NEURONAL NETWORKS BASED ON 5 BIOMARKERS
TAKING RANDOMLY FROM TABLE 1A.

Fructosyllysine (from the biomarker family “Lysine and conjugates”)
Integrin beta
Isobutyrylcarnitine (C4) (from the biomarker family “Carnitine and
conjugates”)
Myosin regulatory light chain RLC-A
Talin 2

| | |
|---|---|
| Accuracy | 78.0% |
| Specificity | 75.4% |
| Sensitivity | 81.6% |

TABLE 10B
EXAMPLE OF PERFORMANCES TO PREDICT
ALZHEIMER'S SILENT PHASE THANKS
TO NEURONAL NETWORKS BASED ON 6 BIOMARKERS
TAKING RANDOMLY FROM TABLE 1A.

Fructosyllysine (from the biomarker family “Lysine and conjugates”)
Igh-6 protein (from the biomarker family “Globulin family”)
Myosin regulatory light chain RLC-A
Octadecanedioate (C18)
Ribonate (ribonolactone)
Talin 2

| | |
|---|---|
| Accuracy | 83.0% |
| Specificity | 84.9% |
| Sensitivity | 81.6% |

TABLE 10C
EXAMPLE OF PERFORMANCES TO PREDICT
ALZHEIMER'S SILENT PHASE THANKS
TO NEURONAL NETWORKS BASED ON 14 BIOMARKERS
TAKING RANDOMLY FROM TABLE 1A.

10 kDa heat shock protein, mitochondrial
5-hydroxylysine (from the biomarker family “Lysine and conjugates”)
Adenylate kinase 4, mitochondrial
Calreticulin
Creatine kinase B-type (from the biomarker family “Creatinine kinase
family”)
Ergothioneine
Peptidyl-prolyl cis-trans isomerase FKBP1A
Fructosyllysine (from the biomarker family “Lysine and conjugates”)
Globin c2 (from the biomarker family “Globin family”)
Integrin subunit alpha V
Myoglobin (from the biomarker family “Globin family”)
Retinoic acid receptor responder 2
Tmprss13 protein
Transferrin receptor protein 1

| | |
|---|---|
| Accuracy | 84.0% |
| Specificity | 84.9% |
| Sensitivity | 81.0% |

TABLE 10D
EXAMPLE OF PERFORMANCES TO PREDICT
ALZHEIMER'S SILENT PHASE THANKS
TO NEURONAL NETWORKS BASED ON 26 BIOMARKERS
TAKING RANDOMLY FROM TABLE 1A.

Rat apolipoprotein E protein (from the biomarker family
“Apolipoproteins”)
Arp2/3 complex 34 kDa subunit (from the biomarker family “Arp2/3
complex proteins”)
Carnitine (from the biomarker family “Carnitine and conjugates”)
Isobutyrylcarnitine (C4) (from the biomarker family “Carnitine and
conjugates”)
Isovalerylcarnitine (C5) (from the biomarker family “Carnitine and
conjugates”)
Coagulation factor VII (from the biomarker family “Coagulation factor
family”)
Serine (or cysteine) proteinase inhibitor clade A member 4 (from the
biomarker family “Globulin family”)
Igh-6 protein (from the biomarker family “Globulin family”)
Serum amyloid P-component
Allantoic acid
Calpain small subunit 1
Carboxypeptidase B2
Carnosine
Clathrin heavy chain
Complement C6
Extracellular matrix protein 1
Fructose-bisphosphate aldolase
Keratin type II cytoskeletal 5
Mannose-binding protein A
Myosin regulatory light chain RLC-A
N-acetylasparagine
Octadecanedioate (C18)
Ribonate (ribonolactone)
Ribulonate
Talin 2
Xaa-Pro aminopeptidase 2

| | |
|---|---|
| Accuracy | 86.0% |
| Specificity | 86.7% |
| Sensitivity | 86.6% |

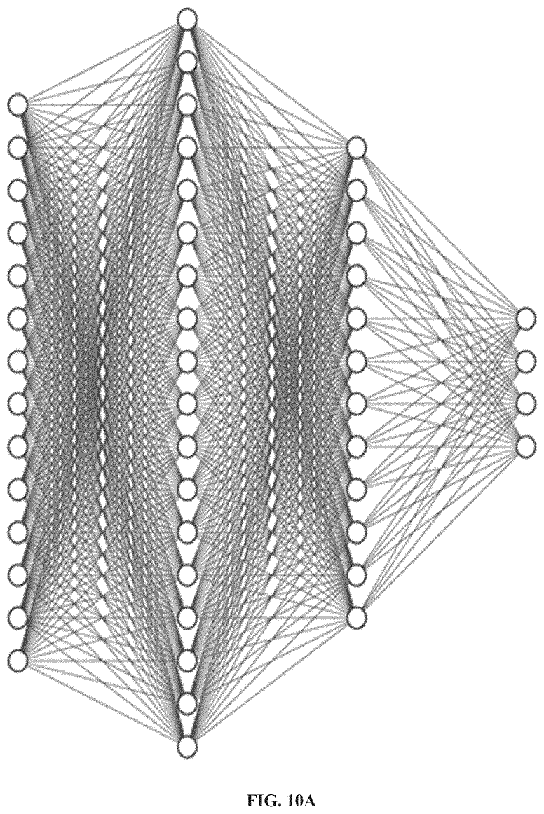

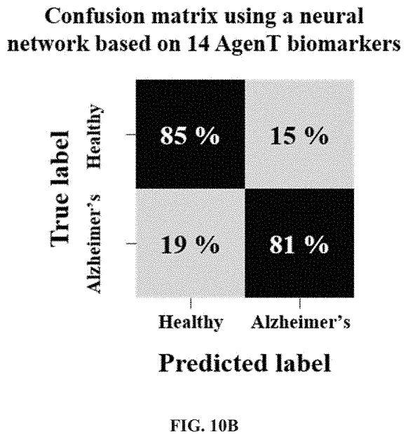

Taking a subset of 14 biomarkers (Tables IOC and 11) and a feed forward neural network—more precisely a multilayer perceptron (FIG. 10A)—with a logistic activation function, we could obtain an accuracy for silent AD detection over 5 cross-validations of 84%, a specificity of 84.9% (true negatives, i.e., healthy subjects identified as such) and a sensitivity of 81% (true positives, i.e., silent AD subjects identified as such) (FIG. 10B).

TABLE 11
SUBSET OF 14 BIOMARKERS SELECTED FROM THE GROUP
OF BIOMARKERS OF TABLE 1A. VARIATIONS OF LEVEL,
AMOUNT OR CONCENTRATION IN EACH OF THE GRADE
S1, S2 AND S3 VERSUS SUBSTANTIALLY HEALTHY.

| | Grade S1 | Grade S2 | Grade S3 |
|---|---|---|---|
| | versus | versus | versus |
| Biomarkers | healthy | healthy | healthy |
| 10 kDa heat shock protein, | −37% | 14% | −43% |
| mitochondrial | | | |
| 5-hydroxylysine (from the biomarker | −48% | −27% | −62% |
| family “Lysine and conjugates”) | | | |
| Adenylate kinase 4, mitochondrial | −25% | 8% | −24% |
| Calreticulin | −40% | −6% | −21% |
| Creatine kinase B-type (from the | 37% | 3% | −32% |
| biomarker family “Creatinine kinase | | | |
| family”) | | | |
| Ergothioneine | −48% | −2% | 122% |
| Fructosyllysine (from the biomarker | 31% | 13% | 131% |
| family “Lysine and conjugates”) | | | |
| Globin c2 (from the biomarker family | −43% | −25% | 32% |
| “Globin family”) | | | |
| Integrin subunit alpha V | −7% | 71% | 8% |
| Myoglobin (from the biomarker | 31% | −43% | 3% |
| family “Globin family”) | | | |
| Peptidyl-prolyl cis-trans isomerase | 78% | 5% | 7% |
| FKBP1A | | | |
| Retinoic acid receptor responder 2 | 9% | 57% | −31% |
| Tmprss13 protein | 59% | 12% | 10% |
| Transferrin receptor protein 1 | −25% | −39% | 67% |

Stratification of the Silent Phase of Alzheimer's Disease

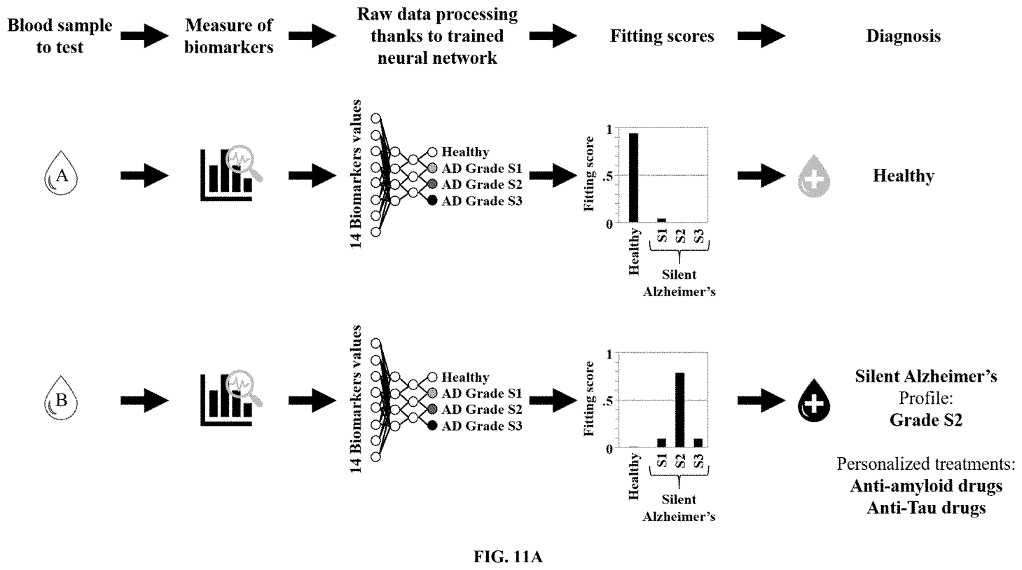

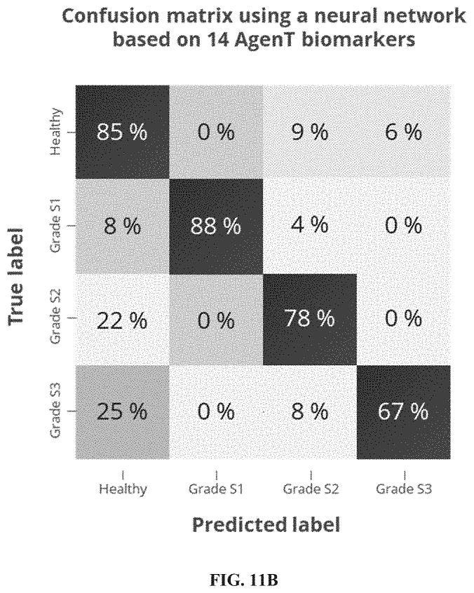

In addition, it is also possible to detect, still using the same 14 randomly selected biomarkers, the stratification of AD, as shown in the confusion matrix in FIG. 11B, still performed using 5 cross-validations.

The stratification method is exemplified in FIG. 11A. To summarize, for a subject to test, blood biomarkers profile is compared with each of the reference signatures or profiles. A “fitting” score is calculated by the trained algorithm based on the percentage of fitting between the tested individual molecular signature or profile and the reference signatures or profiles. The subject is assigned to the stratification (healthy, grade SI, grade S2 or grade S3) with the higher fitting score.

### Example 2: Validation of the 119 Best-In-Class Plasmatic Biomarkers in Human

Material & Methods

By sampling the plasma of a non-transgenic animal model successfully reproducing the continuum of Alzheimer's disease progression at the brain level (Audrain et al, 2018. Cereb Cortex. 28(11):3976-3993), we identified the 119 best-in-class plasmatic biomarkers using artificial intelligence.

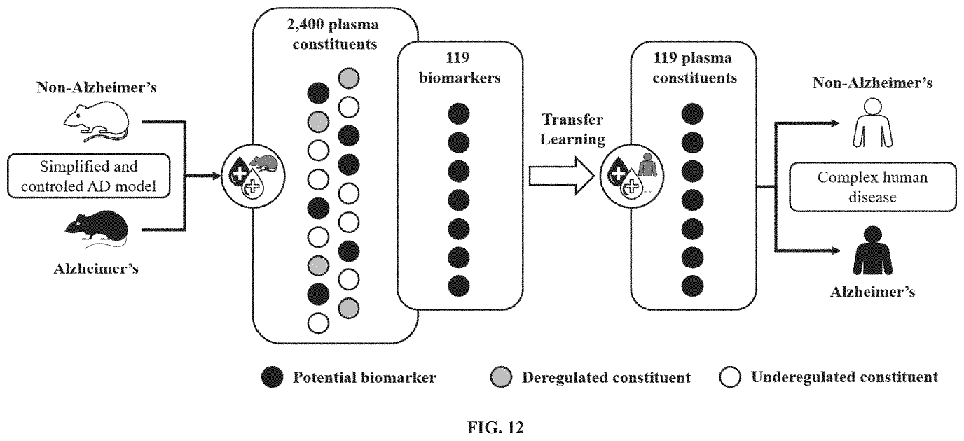

We then analyzed the behavior of these biomarkers in 232 human plasma samples collected up to 13 years before the dementia onset (FIG. 12). Three independent cohorts were used: two with the sporadic form of AD (one from France, one from Spain) and one with Down syndrome individuals (from Spain). Table 12 shows the typology of the tested patients: Alzheimer's patients (including asymptomatic, prodromal and demented patients) and non-Alzheimer's individuals (healthy controls and patients suffering from a neurodegenerative disease excluding AD, such as frontotemporal dementia (FTD),

Lewy body dementia, vascular dementia, psychological disorder, suspected non-Alzheimer disease pathophysiology (SNAP), isolated amyloidosis, primary progressive aphasia, multiple system atrophy, corticobasal degeneration, or mixed dementia) as negative controls. Table 13A-C shows the disease characteristics from the three cohorts.

TABLE 12
TYPOLOGY OF THE TESTED PATIENTS.

| | | II |
|---|---|---|
| Non-AD patients | Healthy controls | 50 |
| | Other dementias | 53 |
| AD patients | Asymptomatic AD | 34 |
| | Prodromal AD | 55 |
| | Dementia phase | 40 |

TABLE 13 A
DISEASE CHARACTERISTICS FROM AD COHORT 1.

| | HC | OD | pAD | AD |
|---|---|---|---|---|
| | (n = 40) | (n = 40) | (n = 35) | (n = 20) |
| Age in years, | 67.0 ± 8.7 | 70.5 ± 7.7 | 71.0 ± 7.7 | 69.0 ± 7.4 |
| median ± SD | | | | |
| MMSE Score, | 27.5 ± 1.4 | 24.0 ± 5.6 | 27.0 ± 1.1 | 14.0 ± 1.3 |
| median ± SD | | | | |
| CSF pTau¹⁸¹/Aβ42, | 0.031 ± 0.014 | 0.038 ± 0.032 | 0.147 ± 0.109 | 0.134 ± 0.076 |
| median ± SD | | | | |

MMSE: mini-mental state examination; HC: healthy controls; OD: other dementias excluding Alzheimer's; pAD: prodromal Alzheimer's disease; AD: Alzheimer's disease; CSF: cerebrospinal fluid.

TABLE 13B
DISEASE CHARACTERISTICS FROM AD COHORT 2.

| | HC | OD | pAD | AD |
|---|---|---|---|---|
| | (n = 10) | (n = 13) | (n = 10) | (n = 10) |
| Age in years, | 60.0 ± 15.3 | 73.0 ± 11.0 | 72.5 ± 5.1 | 75.5 ± 5.5 |
| median ± SD | | | | |
| MMSE Score, | 29.0 ± 0.7 | 22.0 ± 7.8 | 26.0 ± 1.9 | 22.5 ± 7.3 |
| median ± SD | | | | |
| CSF pTau¹⁸¹/Aβ42, | 0.030 ± 0.010 | 0.040 ± 0.008 | 0.184 ± 0.060 | 0.209 ± 0.107 |
| median ± SD | | | | |

MMSE: mini-mental state examination; HC: healthy controls; OD: other dementias excluding Alzheimer's; pAD: prodromal Alzheimer's disease; AD: Alzheimer's disease; CSF: cerebrospinal fluid.

TABLE 13C
DISEASE CHARACTERISTICS FROM DS COHORT.

| | aAD | pAD | AD |
|---|---|---|---|
| | (n = 34) | (n = 10) | (n = 10) |
| Age in years, | 40.0 ± 8.8 | 48.5 ± 3.8 | 49.5 ± 5.6 |
| median ± SD | | | |
| CAMCOG Score, | 75.0 ± 18.6 | 63.0 ± 21.2 | 63.0 ± 14.3 |
| median ± SD | | | |
| CSF pTau¹⁸¹/Aβ42, | 0.020 ± 0.07 | 0.301 ± 0.27 | 0.340 ± 0.10 |
| median ± SD | | | |

CAMCOG: Cambridge cognitive examination; aAD: asymptomatic Alzheimer's disease; pAD: prodromal Alzheimer's disease; AD: Alzheimer's disease; CSF: cerebrospinal fluid.

To confirm the informativity on the biomarkers of Table 1A, we compared them to the rest of the blood constituents (i.e., plasma constituents which are not identified in Table 1A, termed “non-Table 1A constituents” in the following) as follows:

1) We randomly selected a set of “n” biomarkers (with n=2, 5, 15 or 25 biomarkers) from Table 1A and evaluated the performance to detect Alzheimer's patients, on 5 cross-validations with a logistic regression only based on these n biomarkers.

Here, we deliberately used logistic regression because it is a basic classifier that allows to account for the informativity of the randomly selected biomarkers considered by combining them linearly, and therefore, with a reduced risk of overlearning compared to other algorithms performing non-linear combinations such as neural networks.

2) This procedure was performed 250 and 1000 times, and allowed us to obtain an accuracy to detect AD in the asymptomatic phase for each of 5000 randomly selected biomarker sets. We have chosen to carry out two independent runs (250 and 1000 combinations) to demonstrate the robustness of the average performance obtained for 2, 5, 15 and 25 random biomarkers.

3) The same procedure was also performed considering the non-Table 1A constituents. Thus, it is possible to compare the performance of biomarkers of Table 1A and non-Table 1A constituents.

4) We tested the distribution difference using Mann Whitney's nonparametric test or two-way ANOVA. We also set a threshold of 70% of correct diagnosis as a performance threshold for a diagnostic test usable in clinics. We compared the percentage of randomly selected combinations that reach this threshold for Table 1A biomarkers and non-Table 1A constituents.

Results

With 250 random selections

The average performances obtained with combinations of 2, 5, 15 and 25 plasma components are as shown in Table 14.

TABLE 14
AVERAGE PERFORMANCES WITH
250 RANDOM SELECTIONS.

| | Biomarkers of | | | Non-Table 1A | | |
|---|---|---|---|---|---|---|
| Numbers of | Table 1A | | | constituents | | |
| biomarkers | Mean | SD | n | Mean | SD | n |
| 2 | 0.5794 | 0.07932 | 250 | 0.5328 | 0.0598 | 250 |
| 5 | 0.6241 | 0.0688 | 250 | 0.5582 | 0.06239 | 250 |
| 15 | 0.6897 | 0.05221 | 250 | 0.6105 | 0.05118 | 250 |
| 25 | 0.7127 | 0.04423 | 250 | 0.637 | 0.04844 | 250 |

Values in bold indicate a significant difference between Table 1A biomarkers and non-Table 1A constituents (p<0.0001, Mann Whitney's nonparametric test).

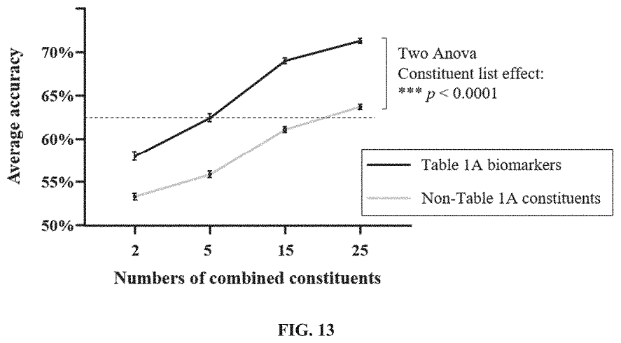

It is important to note that the performance obtained with 5 biomarkers from Table 1A is equivalent to that obtained for 15 and 25 non-Table 1A constituents (FIG. 13).

With 1000 random selections

For 2 random biomarkers

For 1000 random selections, the performance using 2 biomarkers in correctly identifying AD patients is on average 56.92%±0.002% for biomarkers of Table 1A and 53.28%±0.002% for non-Table 1A constituents. This difference is significantly different with a p value<0.0001 (Mann Whitney's nonparametric test).

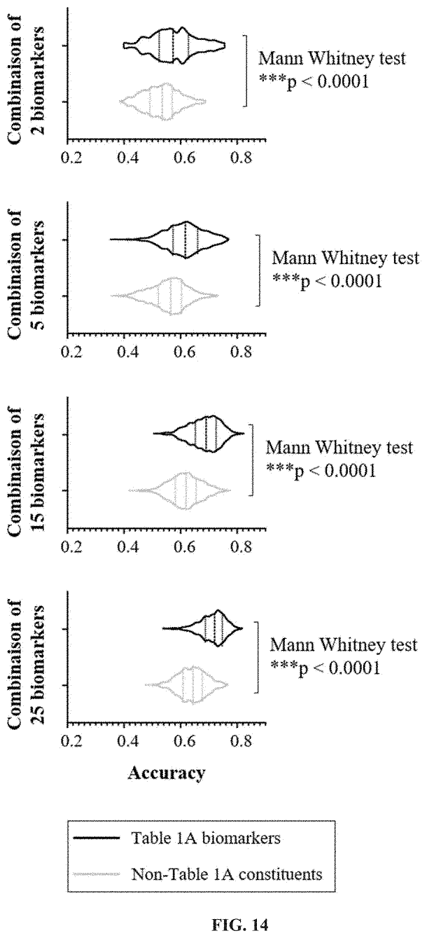

This confirms that whatever 2 random biomarkers taken in Table 1A, the performance obtained will statistically overperform that obtained with 2 random non-Table 1A constituents (FIG. 14).

Having at least 2 biomarkers of Table 1A therefore increases the detection of Alzheimer's disease in the silent phase. This validates the superiority of all 119 biomarkers of Table 1A—when at least 2 are used—to detect patients with AD from the silent phase, over all other plasma constituents.

For 5 random biomarkers

The performance using 5 biomarkers in correctly identifying Alzheimer's patients is on average 61.65%±0.002% for biomarkers of Table 1A and 56.17%±0.002% for non-Table 1A constituents. This difference is significantly different with a p value<0.0001 (Mann Whitney's nonparametric test).

This confirms that whatever 5 random biomarkers taken in Table 1A, the performance obtained will statistically overperform that obtained with 5 random non-Table 1A constituents (FIG. 14).

Having at least 5 biomarkers of Table 1A therefore increases the detection of Alzheimer's disease in the silent phase. This validates the superiority of all 119 biomarkers of Table 1A—when at least 5 are used—to detect patients with AD from the silent phase, over all other plasma constituents.

For 15 random biomarkers

The performance using 15 biomarkers in correctly identifying Alzheimer's patients is on average 68.27%±0.002% for biomarkers of Table 1A and 61.56%±0.002% for non-Table 1A constituents. This difference is significantly different with a p value<0.0001 (Mann Whitney's nonparametric test).

This confirms that whatever 15 random biomarkers taken in Table 1A, the performance obtained will statistically overperform that obtained with 15 random non-Table 1A constituents (FIG. 14). It is also interesting to note that the accuracy obtained with 5 random biomarkers from Table 1A is equivalent to that obtained with 15 non-Table 1A constituents.

Having at least 15 biomarkers of Table 1A therefore increases the detection of Alzheimer's disease in the silent phase. This validates the superiority of all 119 biomarkers of Table 1A—when at least 15 are used—to detect patients with AD from the silent phase, over all other plasma constituents.

For 25 random biomarkers

The performance using 25 biomarkers in correctly identifying Alzheimer's patients is on average 71.47%±0.001% for biomarkers of Table 1A and 64.08%±0.002% for non-Table 1A constituents. This difference is significantly different with a p value<0.0001 (Mann Whitney's nonparametric test).

This confirms that whatever 25 random biomarkers taken in Table 1A, the performance obtained will statistically overperform that obtained with 25 random non-Table 1A constituents (FIG. 14).

Having at least 25 biomarkers of Table 1A therefore increases the detection of Alzheimer's disease in the silent phase. This validates the superiority of all 119 biomarkers of Table 1A—when at least 25 are used—to detect patients with AD from the silent phase, over all other plasma constituents.

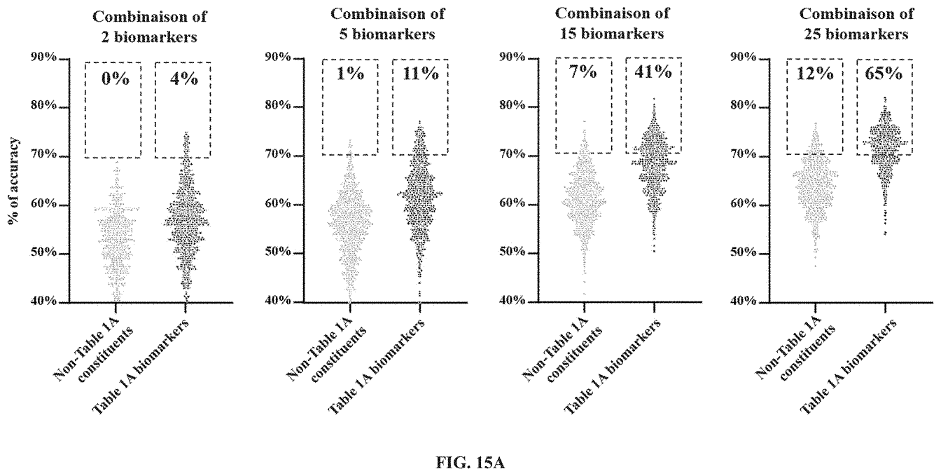

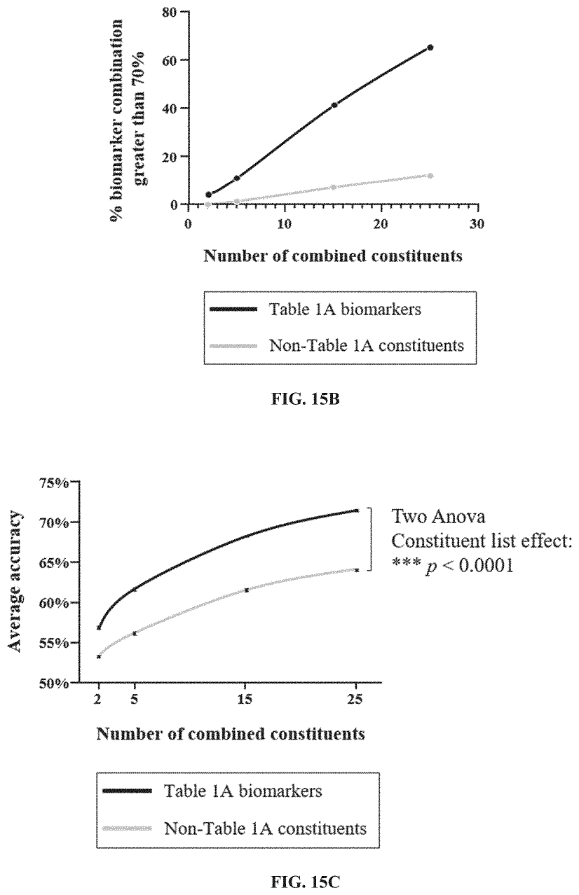

As can be seen on FIG. 15A-B, there is a 65% of chance of achieving an accuracy greater than 70% with 25 biomarkers randomly selected from Table 1A, but only a 12% chance with 25 random non-Table 1A constituents. This increased performance is also observable from 2 biomarkers, so that the threshold of 70% accuracy is reached in 4% of the cases with 2 biomarkers randomly selected from Table 1A, against 0% for non-Table 1A constituents. Using at least 2 biomarkers from Table 1A thus significantly increases the diagnostic performance above 70% compared to the other plasma constituents.

The combined 2-way ANOVA analysis confirms the superiority of the biomarkers of Table 1A over all plasma components (non-Table 1A constituents) to diagnose Alzheimer's patients (FIG. 15C).

It is interesting to note that the performances obtained with 5 randomly selected biomarkers of Table 1A are slightly better than those obtained with 15 non-Table 1A constituents. Once again, these results underline the ability of the biomarkers of Table 1A to identify Alzheimer's patients from non-Alzheimer's individuals.

Controls including patients with other neurodegenerative diseases confirm the specificity of the biomarkers of Table 1A for Alzheimer's disease from its silent phase.

### CONCLUSION

To conclude, we had identified plasma markers in rats, based on their high informative values to identify Alzheimer's rats from control rats (Example 1). This raised the crucial question of the transferability in humans of these biomarkers identified in rats.

Knowing the low relevance of transgenic animal models to identify blood biomarkers for AD, could we demonstrate the superiority of the AgenT rat model to identify biomarkers bearing information on Alzheimer's status from its silent phase?

The analysis of all 119 “best-in-class” biomarkers pre-identified in rats, used in combination and compared to other combined plasma molecules (non-Table 1A constituents), demonstrates the high informative value for all biomarkers of Table 1A.

Example 2 indeed demonstrates that all combinations of biomarkers from Table 1A

provide a clinical diagnostic value that statistically overperforms informative values of the non-Table 1A constituents, from a combination of as low as two biomarkers.

Altogether, these data prove that all biomarkers pre-identified in rats (Table 1A) are informative and specific biomarkers of AD in humans, thereby validating both the AgenT rat model and the developed learning transfer approach used here for the first time.

1.-22. (canceled)

23. A computer system for diagnosing Alzheimer's disease in a subject having, at risk of having, or suspected of having Alzheimer's disease, the computer system comprising:
(i) at least one processor, and
(ii) at least one storage medium that stores at least one code readable by the processor, and which, when executed by the processor, causes the processor to:
a. receive an input level, amount or concentration of at least five biomarkers selected from the group of biomarkers of Table 1A, determined in a sample from a subject having, at risk of having, or suspected of having Alzheimer's disease,
b. analyze and transform the input level, amount or concentration of the at least five biomarkers by organizing and/or modifying each input level, amount or concentration to derive at least one of a probability score, a fitting score and a classification label,
c. generate an output, wherein the output is the at least one of the classification label, the fitting score and the probability score, and
d. provide a diagnosis of the subject as being affected or not with Alzheimer's disease based on the output.

24. The computer system according to claim 23, wherein step b. is via a machine learning algorithm, the machine learning algorithm trained with a training dataset, the training dataset comprising information relating to the level, the amount, or the concentration of the same at least five biomarkers of Table 1A determined in samples from subjects of known Alzheimer's disease status.

25. The computer system according to claim 24, wherein said machine learning algorithm is selected from the group consisting of an artificial neural network (ANN), a perceptron algorithm, a deep neural network, a clustering algorithm, a k-nearest neighbors algorithm (k-NN), a decision tree algorithm, a random forest algorithm, a linear regression algorithm, a linear discriminant analysis (LDA) algorithm, a quadratic discriminant analysis (QDA) algorithm, a support vector machine (SVM), a Bayes algorithm, a simple rule algorithm, a clustering algorithm, a meta-classifier algorithm, a Gaussian mixture model (GMM) algorithm, a nearest centroid algorithm, an extreme gradient boosting (XG Boost) algorithm, a linear mixed effects model algorithm, and a combination thereof.

26. The computer system according to claim 24, wherein the training dataset comprises information relating to the level, amount or concentration of the same at least five biomarkers of Table 1A determined in samples from substantially healthy subjects and from subjects known to be affected with Alzheimer's disease.

27. The computer system according to claim 23, wherein providing a diagnosis at step d. comprises providing a stratification of the subject being affected with Alzheimer's disease into a grade of Alzheimer's disease.

28. The computer system according to claim 23, wherein step a. comprises receiving an input level, amount or concentration of at least 14 biomarkers selected from the group of biomarkers of Table 1A.

29. The computer system according to claim 24, wherein the training dataset comprises information relating to the level, amount or concentration of the same at least five biomarkers of Table 1A determined in samples from substantially healthy subjects and from subjects known to be affected with grades of Alzheimer's disease.

30. The computer system according to claim 23, wherein Alzheimer's disease is a silent phase of Alzheimer's disease.

31. The computer system according to claim 30, wherein the silent phase of Alzheimer's disease is one of asymptomatic phase of Alzheimer's disease and prodromal phase of Alzheimer's disease.

32. The computer system according to claim 23, wherein the subject does not exhibit at least one of mild cognitive impairment (MCI) and dementia.

33. A computer-implemented method for diagnosing Alzheimer's disease in a subject having, at risk of having, or suspected of having Alzheimer's disease, said method comprising:
a. receiving an input level, amount or concentration of at least five biomarkers selected from the group of biomarkers of Table 1A, determined in a sample from a subject having, at risk of having, or suspected of having Alzheimer's disease,
b. analyzing and transforming the input level, amount or concentration of the at least five biomarkers by organizing and/or modifying each input level, amount or concentration to derive at least one of a probability score, a fitting score and a classification label, determined in samples from subjects of known Alzheimer's disease status,
c. generating an output, wherein the output is the at least one of the classification label, the fitting score and the probability score, and
d. providing a diagnosis of the subject as being affected or not with Alzheimer's disease based on the output.

34. The computer-implemented method according to claim 33, wherein step b. is via a machine learning algorithm, the machine learning algorithm trained with a training dataset, the training dataset comprising information relating to the level, the amount, or the concentration of the same at least five biomarkers of Table 1A determined in samples from subjects of known Alzheimer's disease status.

35. The computer-implemented method according to claim 34, wherein said machine learning algorithm is selected from the group consisting of an artificial neural network (ANN), a perceptron algorithm, a deep neural network, a clustering algorithm, a k-nearest neighbors algorithm (k-NN), a decision tree algorithm, a random forest algorithm, a linear regression algorithm, a linear discriminant analysis (LDA) algorithm, a quadratic discriminant analysis (QDA) algorithm, a support vector machine (SVM), a Bayes algorithm, a simple rule algorithm, a clustering algorithm, a meta-classifier algorithm, a Gaussian mixture model (GMM) algorithm, a nearest centroid algorithm, an extreme gradient boosting (XG Boost) algorithm, a linear mixed effects model algorithm, and a combination thereof.

36. The computer-implemented method according to claim 34, wherein the training dataset comprises information relating to the level, amount or concentration of the same at least five biomarkers of Table 1A determined in samples from substantially healthy subjects and from subjects known to be affected with Alzheimer's disease.

37. The computer-implemented method according to claim 33, wherein providing a diagnosis at step d. comprises providing a stratification of the subject being affected with Alzheimer's disease into a grade of Alzheimer's disease.

38. The computer-implemented method according to claim 33, wherein step a. comprises receiving an input level, amount or concentration of at least 14 biomarkers selected from the group of biomarkers of Table 1A.

39. The computer-implemented method according to claim 34, wherein the training dataset comprises information relating to the level, amount or concentration of the same at least five biomarkers of Table 1A determined in samples from substantially healthy subjects and from subjects known to be affected with grades of Alzheimer's disease.

40. The computer-implemented method according to claim 33, wherein Alzheimer's disease is a silent phase of Alzheimer's disease.

41. The computer-implemented method according to claim 40, wherein the silent phase of Alzheimer's disease is one of asymptomatic phase of Alzheimer's disease and prodromal phase of Alzheimer's disease.

42. The computer-implemented method according to claim 33, wherein the subject does not exhibit at least one of mild cognitive impairment (MCI) and dementia.
